(19) United States
(12) Patent Application Publication
(10) Pub. No.: US 2024/0090802 A1
(43) Pub. Date: Mar. 21, 2024

(54) CONTINUOUS ANALYTE SENSOR DEVICES AND METHODS

(71) Applicant: DexCom, Inc., San Diego, CA (US)

(72) Inventors: Devon M. Headen, San Diego, CA (US); Joshua Ray Windmiller, San Diego, CA (US); Avid Najdahmadi, San Diego, CA (US); Sylvie Liong, San Diego, CA (US); Peter C. Simpson, Cardiff, CA (US); Shannon Reuben Woodruff, San Diego, CA (US); Christina Rodriguez, San Diego, CA (US); Jiong Zou, San Diego, CA (US)

(73) Assignee: DexCom, Inc., San Diego, CA (US)

(21) Appl. No.: 18/241,655

(22) Filed: Sep. 1, 2023

Related U.S. Application Data
(60) Provisional application No. 63/403,568, filed on Sep. 2, 2022.
(60) Provisional application No. 63/403,582, filed on Sep. 2, 2022.
(60) Provisional application No. 63/413,867, filed on Oct. 6, 2022.

(51) Int. Cl. A61B 5/1486; A61B 5/145

(52) U.S. Cl. CPC A61B 5/14865; A61B 5/14546

(57) ABSTRACT

A continuous (multi-)analyte sensor device is provided, comprising a (multi-)analyte sensor operably coupled to a signal transducer, the (multi-)analyte sensor comprising at least one membrane, the least one membrane comprising a least one first transducing element, and at least one of a mediator and/or a regenerative cofactor.

### CROSS-REFERENCE TO RELATED APPLICATIONS

This application claims the benefit of U.S. Provisional Application No. 63/403,568 filed on Sep. 2, 2022, U.S. Provisional Application No. 63/403,582 filed on Sep. 2, 2022 and U.S. Provisional Application No. 63/413,867 filed on Oct. 6, 2022, the entirety of each of which is incorporated herein by reference.

### TECHNICAL FIELD

This disclosure is directed to continuous analyte sensor devices and methods as well as continuous multi-analyte sensor devices and methods.

### BACKGROUND

In vivo analyte sensors can typically be configured to analyze a single analyte using an enzyme to provide specificity for the single analyte. Determining concentrations of multiple analytes of physiological relevance can be desirable in certain medical instances. The continuous quantification of circulating analytes has remained a major challenge in clinical medicine.

### SUMMARY

In a first aspect, a continuous (multi-)analyte sensor device is provided, comprising an in-dwelling (multi-)analyte sensor operably coupled to a signal transducer, the (multi-)analyte sensor comprising at least one membrane system adjacent the signal transducer, the least one membrane system comprising, independently: a first layer comprising at least one first transducing element; and a second layer adjacent the first layer, the second layer being the same or different as the first layer.

In one example, the signal transducer comprises at least one electrode. In another example, alone or in combination with any one of the previous examples, the at least one electrode comprises a first working electrode surface and a second working electrode surface that is vertically, horizontally, or circumferentially spatially separated from the first working electrode surface.

In another example, alone or in combination with any one of the previous examples, the first layer is adjacent to the first working electrode surface. In another example, alone or in combination with any one of the previous examples, the second layer is adjacent to the second working electrode surface. In another example, alone or in combination with any one of the previous examples, at least a portion of the first layer is adjacent to at least a portion of the first working electrode surface and at least a portion of the second layer is distally, vertically, horizontally, or circumferentially separated from at least a portion of the first working electrode surface than the portion of the first layer.

In another example, alone or in combination with any one of the previous examples, second layer comprises at least one second transducing element, the second transducing element being different from the first transducing element.

In another example, alone or in combination with any one of the previous examples, the device further comprises at least one of a mediator and a regenerative cofactor present in the first layer, the second layer, or in both the first and second layers, wherein the at least one the mediator is operatively associated with the at least one first transducing element or the at least one second transducing element.

In another example, alone or in combination with any one of the previous examples, the first layer comprises the at least one mediator and the at least one first transducing element, and the second layer comprises at least one regenerative cofactor.

In another example, alone or in combination with any one of the previous examples, the second layer comprises the at least one second transducing element, and where at least a portion of the first layer is proximal to the first working electrode surface and at least a portion of the second layer is distal from the first working electrode surface.

In another example, alone or in combination with any one of the previous examples, the first layer comprises the at least one regenerative cofactor and the at least one first transducing element and the second layer comprises the at least one mediator.

In another example, alone or in combination with any one of the previous examples, the second layer comprises the at least one second transducing element, wherein at least a portion of the first layer is proximal to the first working electrode surface and at least a portion of the second layer is distal from the first working electrode surface.

In another example, alone or in combination with any one of the previous examples, the first layer comprises the at least one mediator, the at least one regenerative cofactor, and the at least one first transducing element, and the second layer comprises the at least one second transducing element, wherein at least a portion of the first layer is proximal to the first working electrode surface and at least a portion of the second layer is distal from the first working electrode surface.

In another example, alone or in combination with any one of the previous examples, the first layer comprises the at least one first transducing element, and the second layer comprises the at least one regenerative cofactor, at least one mediator, and the at least one second transducing element, where at least a portion of the first layer being proximal to the first working electrode surface and at least a portion of the second layer being distal from the first working electrode surface.

In another example, alone or in combination with any one of the previous examples, the first layer comprises the at least one mediator and the at least one first transducing element, and the second layer comprises the at least one regenerative cofactor.

In another example, alone or in combination with any one of the previous examples, the first layer comprises the at least one regenerative cofactor and the at least one first transducing element and the second layer comprises the at least one mediator.

In another example, alone or in combination with any one of the previous examples, the first layer comprises the at least one mediator, the at least one regenerative cofactor, and the at least one first transducing element and the second layer comprises the at least one second transducing element, wherein at least a portion of the first layer is proximal to the first working electrode surface and at least a portion of the second layer is proximal to the second working electrode surface.

In another example, alone or in combination with any one of the previous examples, the first layer comprises the at least one first transducing element and the second layer comprises the at least one regenerative cofactor, at least one mediator, and the at least one second transducing element, wherein at least a portion of the first layer is proximal to the first working electrode surface and at least a portion of the second layer is proximal to the second working electrode surface.

In another example, alone or in combination with any one of the previous examples, the at least one regenerative cofactor is one or more of NAD, NADH, NAD(P)H, NAD(P)+, mutant NAD+(nox) (or NADH oxidase or NADHox, ATP, flavin adenine dinucleotide (FAD), magnesium (Mg++), pyrroloquinoline quinone (PQQ), pyrroloquinoline quinone (PQQ), and functionalized derivatives thereof.

In another example, alone or in combination with any one of the previous examples, the mediator or the at least one regenerative cofactor is covalently, electrostatically, ionically associated with or physically entrapped or absorbed by one or more of the first layer, the second layer, or the third layer. In another example, alone or in combination with any one of the previous examples, the mediator or the at least one regenerative cofactor is covalently, electrostatically, ionically associated with, or physically entrapped, or absorbed by the one or more working electrode surfaces.

In another example, alone or in combination with any one of the previous examples, the second layer comprises the at least one second transducing element, wherein at least a portion of the first layer is proximal to the first working electrode surface and at least a portion of the second layer is proximal to the second working electrode surface.

In another example, alone or in combination with any one of the previous examples, the second layer comprises the at least one second transducing element, wherein at least a portion of the first layer is proximal to the first working electrode surface and at least a portion of the second layer is proximal to the second working electrode surface.

In another example, alone or in combination with any one of the previous examples, the first layer or the second layer comprises a polymer. In another example, alone or in combination with any one of the previous examples, the polymer is polyolefin, polystyrene, polyoxymethylene, polysiloxane, polyether, polyacrylate, polymethacrylate, polyester, polycarbonate, polyamide, polypyridine, poly(pyridine-styrene) copolymer, poly(ether ketone), poly(ether imide), polyurethane, polyurethane urea, polycarbonate-polyurethane copolymer, or blends thereof.

In another example, alone or in combination with any one of the previous examples, the device further comprises at least one third transducing element, the at least one third transducing element present in at least one of the first layer, the second layer and a third layer.

In another example, alone or in combination with any one of the previous examples, the first layer has a first diffusion resistance to one or more analytes or one or more substrate reaction products and the second layer has a second diffusion resistance to the one or more analytes or the one or more enzymatic substrate reaction products, wherein the second diffusion resistance is substantially identical or different from the first diffusion resistance to the one or more analytes or the one or more enzymatic substrate reaction products.

In another example, alone or in combination with any one of the previous examples, the one or more analytes comprises glucose, lactose, glycerol, beta hydroxybutyrate, creatinine, creatine, alcohol, urea, uric acid, cholesterol, bilirubin, glutathione, urea, sodium, potassium, or glutamate.

In another example, alone or in combination with any one of the previous examples, the at least one first transducing element and the at least one second transducing element are independently selected from a dehydrogenase enzyme, a reductase enzyme, a kinase enzyme, a peroxidase enzyme, an esterase enzyme, an (amido)hydrolase enzyme, and an oxidase enzyme.

In another example, alone or in combination with any one of the previous examples, the at least one first transducing element and the at least one second transducing element each have a substrate and produce a reaction product with said substrate and provide a clinical value correlated with a health condition of a mammal. In another example, alone or in combination with any one of the previous examples, the substrate or the reaction product is selected from hydrogen peroxide, creatine, acetoacetate, dihydroxyacetone, oxygen, or an aldehyde.

In another example, alone or in combination with any one of the previous examples, the at least one first transducing element and the at least one second transducing element are beta-hydroxybutyrate dehydrogenase, alcohol dehydrogenase, lipase, amidohydrolase, glycerol kinase, creatinine kinase, creatine amidohydrolase, alcohol oxidase, cholesterol oxidase, galactose oxidase, choline oxidase, glutamate oxidase, glycerol-3-phosphate oxidase, bilirubin oxidase, ascorbic acid oxidase, uric acid oxidase, urease, pyruvate oxidase, xanthine oxidase, glucose oxidase, lactate oxidase, sarcosine oxidase, malate dehydrogenase, formaldehyde dehydrogenase, glutathione reductase, glutathione peroxidase, 3-hydroxy steroid dehydrogenases, or horseradish peroxidase.

In another example, alone or in combination with any one of the previous examples, the mediator is one or more of 2,2′-bipryidine, poly-1,10-phenanthroline-5,6-dione, polyvinylferrocene, hexacyanoferrate, phthalocyanine or organometallic compounds thereof, organometallic compounds of osmium or ruthenium, and functionalized derivatives thereof, and complexes of one or more transition metals with one or more polymers or ligands and salts thereof.

In another example, alone or in combination with any one of the previous examples, the device further comprises a transmitter configured to wireless transmit data to a paired display device or therapeutic delivery device such that multiple analyte parameters are assessed in parallel.

In a second aspect, a continuous analyte sensor device is provided, comprising an in-dwelling mediated analyte sensor operably coupled to a signal transducer, the mediated analyte sensor comprising at least one membrane system adjacent the signal transducer, the least one membrane system comprising, independently: a first layer comprising at least one first transducing element; a second layer adjacent the first layer, the second layer being the same or different as the first layer; at least one mediator; and a mediated system interference domain comprising at least one oxidase enzyme, at least one peroxidase enzyme, catalase, or combination thereof.

In one example, the in-dwelling mediated analyte sensor is a (multi-)analyte sensor. In another example, alone or in combination with any one of the previous examples, the in-dwelling mediated analyte sensor is a glucose and ketone analyte sensor. In another example, alone or in combination with any one of the previous examples, the in-dwelling mediated analyte sensor is a glucose and creatinine analyte sensor. In another example, alone or in combination with any one of the previous examples, the in-dwelling mediated analyte sensor is a glucose and alcohol analyte sensor.

In another example, alone or in combination with any one of the previous examples, the signal transducer comprises at least one electrode. In another example, alone or in combination with any one of the previous examples, the at least one electrode comprises a first working electrode surface and a second working electrode surface that is vertically, horizontally, or circumferentially spatially separated from the first working electrode surface.

In another example, alone or in combination with any one of the previous examples, the first layer is adjacent to the first working electrode surface. In another example, alone or in combination with any one of the previous examples, the second layer is adjacent to the second working electrode surface.

In another example, alone or in combination with any one of the previous examples, at least a portion of the first layer is adjacent to at least a portion of the first working electrode surface and at least a portion of the second layer is distally, vertically, horizontally, or circumferentially separated from at least a portion of the first working electrode surface than the portion of the first layer.

In another example, alone or in combination with any one of the previous examples, the second layer comprises the at least one second transducing element, and where at least a portion of the first layer is proximal to the first working electrode surface and at least a portion of the second layer is distal from the first working electrode surface.

In another example, alone or in combination with any one of the previous examples, the second layer comprises the at least one second transducing element, and where at least a portion of the first layer is proximal to the first working electrode surface and at least a portion of the second layer is distal from the first working electrode surface.

In another example, alone or in combination with any one of the previous examples, the first layer comprises at least one first transducing element and the second layer comprises at least one second transducing element, the second transducing element being different from the first transducing element.

In another example, alone or in combination with any one of the previous examples, the at least one first transducing element and the at least one second transducing element each have a substrate and produce a reaction product with said substrate and provide a clinical value correlated with a health condition of a mammal.

In another example, alone or in combination with any one of the previous examples, the device further comprises at least one regenerative cofactor present in the first layer, the second layer, or in both the first and second layers.

In another example, alone or in combination with any one of the previous examples, the first layer comprises the at least one mediator and the at least one first transducing element, and the second layer comprises the at least one regenerative cofactor.

In another example, alone or in combination with any one of the previous examples, the first layer comprises the at least one regenerative cofactor and the at least one first transducing element and the second layer comprises the at least one mediator.

In another example, alone or in combination with any one of the previous examples, the first layer comprises the at least one mediator, the at least one regenerative cofactor, and the at least one first transducing element, and the second layer comprises the at least one second transducing element, wherein at least a portion of the first layer is proximal to the first working electrode surface and at least a portion of the second layer is distal from the first working electrode surface.

In another example, alone or in combination with any one of the previous examples, the first layer comprises the at least one mediator, the at least one regenerative cofactor, and the at least one first transducing element and the second layer comprises the at least one second transducing element, wherein at least a portion of the first layer is proximal to the first working electrode surface and at least a portion of the second layer is proximal to the second working electrode surface.

In another example, alone or in combination with any one of the previous examples, the first layer comprises the at least one first transducing element, and the second layer comprises the at least one regenerative cofactor, at least one mediator, and the at least one second transducing element, where at least a portion of the first layer being proximal to the first working electrode surface and at least a portion of the second layer being distal from the first working electrode surface.

In another example, alone or in combination with any one of the previous examples, the first layer comprises the at least one first transducing element and the second layer comprises the at least one regenerative cofactor, at least one mediator, and the at least one second transducing element, wherein at least a portion of the first layer is proximal to the first working electrode surface and at least a portion of the second layer is proximal to the second working electrode surface.

In another example, alone or in combination with any one of the previous examples, the second layer comprises the at least one second transducing element, and wherein at least a portion of the first layer is proximal to the first working electrode surface and at least a portion of the second layer is distal from the first working electrode surface.

In another example, alone or in combination with any one of the previous examples, the second layer comprises the at least one second transducing element, wherein at least a portion of the first layer is proximal to the first working electrode surface and at least a portion of the second layer is proximal to the second working electrode surface.

In another example, alone or in combination with any one of the previous examples, the second layer comprises the at least one second transducing element, wherein at least a portion of the first layer is proximal to the first working electrode surface and at least a portion of the second layer is proximal to the second working electrode surface.

In another example, alone or in combination with any one of the previous examples, the device further comprises a third layer adjacent the second layer. In another example, alone or in combination with any one of the previous examples, the device further comprises at least one third transducing element, the at least one third transducing element present in at least one of the first layer, the second layer and the third layer.

In another example, alone or in combination with any one of the previous examples, the first layer has a first diffusion resistance to one or more analytes or one or more substrate reaction products and the second layer has a second diffusion resistance to the one or more analytes or the one or more enzymatic substrate reaction products, wherein the second diffusion In another example, alone or in combination with any one of the previous examples, In another example, alone or in combination with any one of the previous examples, resistance is substantially identical or different from the first diffusion resistance to the one or more analytes or the one or more enzymatic substrate reaction products.

In another example, alone or in combination with any one of the previous examples, the at least one first transducing element and the at least one second transducing element are independently a dehydrogenase enzyme, a reductase enzyme, a kinase enzyme, a peroxidase enzyme, an esterase enzyme, an (amido)hydrolase enzyme, an oxidase enzyme, or combinations thereof.

In another example, alone or in combination with any one of the previous examples, the at least one first transducing element and the at least one second transducing element are independently beta-hydroxybutyrate dehydrogenase, alcohol dehydrogenase, lipase, amidohydrolase, glycerol kinase, creatinine kinase, creatine amidohydrolase, alcohol oxidase, cholesterol oxidase, galactose oxidase, choline oxidase, glutamate oxidase, glycerol-3-phosphate oxidase, bilirubin oxidase, urease, pyruvate oxidase, xanthine oxidase, glucose oxidase, lactate oxidase, sarcosine oxidase, malate dehydrogenase, formaldehyde dehydrogenase, glutathione reductase, glutathione peroxidase, 3-hydroxy steroid dehydrogenases, or combinations thereof.

In another example, alone or in combination with any one of the previous examples, the mediator is one or more of 2,2′-bipryidine, poly-1,10-phenanthroline-5,6-dione, polyvinylferrocene, hexacyanoferrate, phthalocyanine or organometallic compounds thereof, organometallic compounds of osmium or ruthenium, and functionalized derivatives thereof, and complexes of one or more transition metals with one or more polymers or ligands and salts thereof.

In another example, alone or in combination with any one of the previous examples, the at least one oxidase enzyme is ascorbic acid oxidase or uric acid oxidase. In another example, alone or in combination with any one of the previous examples, the at least one peroxidase enzyme is horseradish peroxidase.

In another example, alone or in combination with any one of the previous examples, the mediated system interference domain comprises at least one polymer. In another example, alone or in combination with any one of the previous examples, the at least one polymer is polyolefin, polystyrene, polyoxymethylene, polysiloxane, polyether, polyacrylate, polymethacrylate, polyester, polycarbonate, polyamide, polypyridine, poly(pyridine-styrene) copolymer, poly(ether ketone), poly(ether imide), polyurethane, polyurethane urea, polycarbonate-polyurethane copolymer, poly ethylene vinyl acetate, or blends thereof.

In another example, alone or in combination with any one of the previous examples, the device further comprises at least one of an electrode domain, an enzyme domain, a resistance domain, and an interference membrane.

In another example, alone or in combination with any one of the previous examples, the mediated system interference domain is present in the electrode domain. In another example, alone or in combination with any one of the previous examples, the mediated system interference domain is directly adjacent the electrode domain. In another example, alone or in combination with any one of the previous examples, the mediated system interference domain is present in the enzyme domain. In another example, alone or in combination with any one of the previous examples, the mediated system interference domain is directly adjacent the enzyme domain. In another example, alone or in combination with any one of the previous examples, the mediated system interference domain is present in the resistance domain. In another example, alone or in combination with any one of the previous examples, the mediated system interference domain is directly adjacent the resistance domain. In another example, alone or in combination with any one of the previous examples, the mediated system interference domain is present in the interference membrane. In another example, alone or in combination with any one of the previous examples, the mediated system interference domain is directly adjacent the interference membrane.

In another example, alone or in combination with any one of the previous examples, the device further comprises a transmitter configured to wireless transmit data to a paired display device or therapeutic delivery device such that at least two analyte parameters are assessed in parallel.

In one example, a continuous analyte sensor device is provide, the device comprising an analyte sensor operably coupled to a signal transducer, the analyte sensor comprising at least one membrane system adjacent the signal transducer, the least one membrane system comprising, independently: a first domain comprising at least one first transducing element; a second domain adjacent the first domain, the second domain being the same or different as the first domain; and at least one regenerative cofactor.

In one aspect, the analyte sensor is a multi-analyte sensor. In one aspect, alone or in combination with any of the previous aspects, the multi-analyte analyte sensor is a glucose and ketone analyte sensor, or a glucose and creatinine analyte sensor, or a ketone and potassium ion analyte sensor.

In one aspect, alone or in combination with any of the previous aspects, the signal transducer comprises at least one electrode.

In one aspect, alone or in combination with any of the previous aspects, the first domain comprises at least one first transducing element and the second domain comprises at least one second transducing element, the second transducing element being different from the first transducing element.

In one aspect, alone or in combination with any of the previous aspects, the at least one regenerative cofactor is one or more of NAD, NADH, NAD(P)H, NAD(P)+, ATP, flavin adenine dinucleotide (FAD), magnesium (Mg++), pyrroloquinoline quinone (PQQ), pyrroloquinoline quinone (PQQ), and functionalized derivatives thereof.

In one aspect, alone or in combination with any of the previous aspects, the device further comprises at least one mediator present in the first domain, the second domain, or in both the first and second domains. In one aspect, alone or in combination with any of the previous aspects, the mediator is one or more of 2,2′-bipryidine, poly-1,10-phenanthroline-5,6-dione, polyvinylferrocene, hexacyanoferrate, phthalocyanine or organometallic compounds thereof, organometallic compounds of osmium or ruthenium, and functionalized derivatives thereof, and complexes of one or more transition metals with one or more polymers or ligands and salts thereof.

In one aspect, alone or in combination with any of the previous aspects, the first domain comprises the at least one mediator and the at least one first transducing element, and the second domain comprises the at least one regenerative cofactor, or wherein the first domain comprises the at least one regenerative cofactor and the at least one first transducing element and the second domain comprises the at least one mediator.

In one aspect, alone or in combination with any of the previous aspects, the at least one first transducing element and the at least one second transducing element are independently a dehydrogenase enzyme, a reductase enzyme, a kinase enzyme, a peroxidase enzyme, an esterase enzyme, an (amido)hydrolase enzyme, an oxidase enzyme, or combinations thereof.

In one aspect, alone or in combination with any of the previous aspects, the at least one first transducing element and the at least one second transducing element are independently beta-hydroxybutyrate dehydrogenase, alcohol dehydrogenase, lipase, amidohydrolase, glycerol kinase, creatinine kinase, creatine amidohydrolase, alcohol oxidase, cholesterol oxidase, galactose oxidase, choline oxidase, glutamate oxidase, glycerol-3-phosphate oxidase, bilirubin oxidase, urease, pyruvate oxidase, xanthine oxidase, glucose oxidase, lactate oxidase, sarcosine oxidase, malate dehydrogenase, formaldehyde dehydrogenase, glutathione reductase, glutathione peroxidase, 3-hydroxy steroid dehydrogenases, NADH oxidase, or combinations thereof.

In one aspect, alone or in combination with any of the previous aspects, the at least one first transducing element is beta-hydroxybutyrate dehydrogenase, and the at least one second transducing element is NADH oxidase.

In one aspect, alone or in combination with any of the previous aspects, the at least one electrode comprises platinum or palladium, an interference domain is deposited on the at least one electrode; the first domain is adjacent the interference domain, the first domain comprising beta-hydroxybutyrate dehydrogenase, NADH oxidase, and the cofactor; and the second domain adjacent the first domain, the second domain comprising poly vinylpyridine polymer or copolymer. In one aspect, alone or in combination with any of the previous aspects, the continuous analyte sensor device is configured to provide a continuous analyte signal without a transition metal-containing mediator.

In one aspect, alone or in combination with any of the previous aspects, the at least one regenerative cofactor is NAD.

In one aspect, alone or in combination with any of the previous aspects, the interference domain is configured to block diffusion of at least one of acetaminophen, ascorbic acid, bilirubin, cholesterol, creatinine, dopamine, ephedrine, ibuprofen, L-dopa, methyldopa, salicylate, tetracycline, tolazamide, tolbutamide, triglycerides, and uric acid from the electrode.

In one aspect, alone or in combination with any of the previous aspects, the interference domain comprises polyurethanes, polyurethane-zwitterion polymers, polymers having pendant ionic groups, NAFION™, chitosan, cellulose, alternating layers of polyallylamine and polyacrylate acid or combinations or blends thereof.

In one aspect, alone or in combination with any of the previous aspects, the first domain or the second domain comprises an amphiphilic polymer or copolymer.

In one aspect, alone or in combination with any of the previous aspects, the first domain or the second domain comprises a heterocyclic polymer or copolymer, or an at least partially quarternized heterocyclic polymer or copolymer.

In one aspect, alone or in combination with any of the previous aspects, the device further comprises a transmitter configured to wireless transmit data to a paired display device or therapeutic delivery device.

### BRIEF DESCRIPTION OF THE DRAWINGS

In order to understand and to see how the present disclosure may be carried out in practice, examples will now be described, by way of non-limiting examples only, with reference to the accompanying drawings, in which:

FIGS. 1A-1B depict exemplary enzyme domain configurations for a continuous multi-analyte sensor as disclosed and described herein.

FIGS. 1C-1D depict exemplary enzyme domain configurations for a continuous multi-analyte sensor as disclosed and described herein.

FIG. 1E depicts an exemplary enzyme domain configurations for a continuous multi-analyte sensor as disclosed and described herein.

FIG. 1F depicts exemplary experimental data of an enzyme domain configuration of a continuous multi-analyte sensor as disclosed and described herein.

FIG. 1G depicts linear regression of the experimental data of FIG. 1E.

FIG. 1H depicts exemplary experimental data of a cofactor/enzyme resistance domain configuration of a continuous analyte sensor as disclosed and described herein.

FIG. 1I depicts exemplary experimental data of a cofactor/enzyme resistance domain configuration of a continuous analyte sensor as disclosed and described herein.

FIG. 1J depicts exemplary experimental data of a cofactor/enzyme resistance domain configuration of a continuous analyte sensor as disclosed and described herein.

FIG. 2 depicts exemplary experimental data of an enzyme domain configuration of a continuous multi-analyte sensor as disclosed and described herein.

FIG. 3A depicts exemplary experimental data of an enzyme domain configuration of a continuous multi-analyte sensor as disclosed and described herein.

FIG. 3B depicts exemplary experimental data of the enzyme domain configuration of the continuous multi-analyte sensor of FIG. 3A.

FIG. 3C depicts a series of exemplary experimental data of an enzyme domain configuration of a continuous multi-analyte sensor using different electrode compositions as disclosed and described herein.

FIG. 4 depicts exemplary experimental data of an enzyme domain configuration of a continuous multi-analyte sensor as disclosed and described herein.

FIG. 5 depicts a series of exemplary experimental data of an enzyme domain configuration of a continuous multi-analyte sensor as disclosed and described herein.

FIGS. 6A-6B depict alternative views of an exemplary dual electrode enzyme domain configuration for a continuous multi-analyte sensor as disclosed and described herein.

FIGS. 6C-6D depict alternative views of an exemplary dual electrode enzyme domain configuration for a continuous multi-analyte sensor as disclosed and described herein.

FIG. 6E depicts an exemplary dual electrode configuration for a continuous multi-analyte sensor as disclosed and described herein.

FIG. 6F depicts exemplary experimental data of the dual electrode configuration of the continuous multi-analyte sensor of FIG. 6A-B.

FIG. 7A depicts an exemplary enzyme domain configurations for a continuous multi-analyte sensor as disclosed and described herein.

FIG. 7B depicts exemplary experimental data of the enzyme domain configuration of FIG. 7A.

FIGS. 7C, 7D depict alternative exemplary enzyme domain configurations for a continuous multi-analyte sensor as disclosed and described herein.

FIG. 7E depicts exemplary experimental data of the enzyme domain configuration of FIG. 7D.

FIG. 8A depicts an exemplary enzyme domain configurations for a continuous multi-analyte sensor as disclosed and described herein.

FIGS. 8B, 8C depict exemplary experimental data of the enzyme domain configuration of FIG. 8A.

FIGS. 9A-9D depict alternative views of exemplary dual electrode enzyme domain configurations G1-G4 for a continuous multi-analyte sensor as disclosed and described herein.

FIG. 9E depicts linear regressions of the experimental data of continuous multi-analyte sensor depicted in FIG. 9C

FIG. 10A is a cross-sectional/side-view schematic illustrating an in vivo portion of an analyte sensor, as disclosed herein.

FIG. 10B is a perspective-view schematic illustrating an in vivo portion of a continuous multi-analyte sensor as disclosed and described herein.

FIG. 10C is a side-view schematic illustrating an in vivo portion of a continuous multi-analyte sensor as disclosed and described herein.

FIG. 10D is a cross-sectional/side-view schematic illustrating an in vivo portion of a continuous multi-analyte sensor as disclosed and described herein.

FIG. 10E is a cross-sectional schematic illustrating an in vivo portion of a continuous multi-analyte sensor as disclosed and described herein.

FIG. 10F is a side-view schematic illustrating an in vivo portion of a continuous multi-analyte sensor as disclosed and described herein.

FIG. 10G is a side-view schematic illustrating an in vivo portion of a continuous multi-analyte sensor as disclosed and described herein.

FIG. 11 is a cross-sectional schematic of FIG. 10A, taken on line 11-11, showing an exemplary continuous multi-analyte sensor membrane configuration as disclosed and described herein.

FIG. 12A is a perspective-view schematic illustrating an in vivo portion of a continuous analyte sensor as disclosed and described herein.

FIG. 12B is a perspective-view schematic illustrating an in vivo portion of a continuous analyte sensor as disclosed and described herein.

FIG. 13A is a perspective-view schematic illustrating an in vivo portion of a continuous multi-electrode, multi-analyte sensor.

FIG. 13B is an expanded perspective schematic of section 13B the distal portion of the sensor example illustrated in FIG. 13A.

FIG. 14 depicts a basic schematic of an operating principle of an amperometric enzymatic multi-analyte sensors as disclosed and described herein.

FIG. 15 is a diagram illustrating certain embodiments of an example continuous multianalyte sensor system communicating with at least one display device in accordance with various technologies as disclosed and described herein.

FIG. 16 depicts exemplary experimental calibration data of a mediated continuous analyte sensor as disclosed and described herein.

FIG. 17 depicts exemplary experimental drift data of the continuous analyte sensor of FIG. 16 as disclosed and described herein.

FIG. 18 depicts exemplary experimental in vivo data of the continuous analyte sensor of FIG. 16 as disclosed and described herein.

FIG. 19 depicts exemplary experimental calibration data of a non-mediated continuous analyte sensor as disclosed and described herein.

FIG. 20 depicts exemplary experimental drift data of the continuous analyte sensor of FIG. 19 as disclosed and described herein.

FIGS. 21A, 21B, and 21C depict exemplary continuous ketone sensor configuration pathways as disclosed and described herein.

### DETAILED DESCRIPTION

A multi-analyte sensor designed for in vivo applications, is provided. Multi-analyte sensing may be employed to help diagnose and/or monitor various health conditions, including chronic conditions. In some examples, continuous multi-analyte sensors are configured to measure two or more analytes to enable early intervention for adverse health conditions, including metabolic diseases, as well as to treat health conditions. A multi-analyte sensor has the advantage where in certain instances a single analyte is not able to provide the sufficient information to make decisions regarding whole body health. By allowing for sensing of more than one analyte, the body's biological context is more accurately measured. For example, glucose levels often indicate a certain set of conditions that can be further refined using other analytes. Examples are DKA, metabolic function, insulin sensitivity and clearance, liver function, etc. Diabetic ketoacidosis (DKA) is the leading cause of mortality among individuals with Type 1 diabetes mellitus under the age of 20. Body-adorned continuous glucose monitors have been commercially available over the past two decades for the assessment of interstitial glucose levels. However, in some instances this single-analyte measurement alone may not be clinically sufficient in identifying instances of, for example, euglycemic DKA, which is of growing concern with recent off-label use of SGLT-2 inhibitors among patients undergoing intensive insulin therapies.

The systems and methods discussed herein use multi-analyte sensing to provide feedback to patients and healthcare providers regarding their patients' health.

In one example, a single wire electrode implementation supporting amperometric measurements of multiple analytes is provided.

In another example, a dual coaxial wire electrode implementation supporting amperometric measurements of two or more analytes according to the generalized n-dimensional Cottrell relation:

$$i ( t ) = z F A D {C}^{b , 0} \frac{{e}^{\frac{zF}{R T} Δφ}}{{( 4 π Dt )}^{n / 2}}$$

is employed, where i refers to the electrical current supported by the amperometric reaction, z represents the is the valency or stoichiometric number of electrons partaking in the reaction (e.g., 2 for hydrogen peroxide oxidation), F is Faraday's constant, A is the electrode redox-active surface area, D is the diffusion coefficient of the redox species of interest (e.g., hydrogen peroxide), C^{b,0} is the bulk concentration of the redox species at the electrode surface, R is the universal gas constant, T is the operating temperature (in K), Δϕ is the applied overpotential in volts, t is time measured from the initial application of the overpotential, and n is the dimensionality of the system (e.g., 1, 2, 3). In vivo analyte sensors, for practical applications, tend to be highly diffusion limited such that the governing rate of the reaction (and hence the current) is dominated by the mass diffusivity of the redox species of interest. Owing to the diffusion limitation imparted by an outer resistance domain, an amperometric sensor is designed to control the mass diffusivity of the analyte of interest while attenuating (or enhancing) other co-circulating species.

In another example, the multi-analyte sensor device comprises a transmitter configured to interface with a multi-analyte sensor. In one aspect, wireless transmission of data to a paired display device such that multiple analytes can be assessed in parallel is provided.

The following description and figures illustrate examples of the present disclosure in detail. Those of skill in the art will recognize that there are numerous variations and modifications of this disclosure that are encompassed by its scope. Accordingly, the description examples herein should not be deemed to limit the scope of the present disclosure. In order to facilitate an understanding of the examples disclosed herein, a number of terms are defined below.

The term “about” as used herein is a broad term, and is to be given its ordinary and customary meaning to a person of ordinary skill in the art (and is not to be limited to a special or customized meaning), and refers without limitation to allowing for a degree of variability in a value or range, for example, within 10%, within 5%, or within 1% of a stated value or of a stated limit of a range, and includes the exact stated value or range.

The term “adhere” and “attach” as used herein are broad terms, and are to be given their ordinary and customary meaning to a person of ordinary skill in the art (and are not be limited to a special or customized meaning), and refer without limitation to hold, bind, or stick, for example, by gluing, bonding, grasping, interpenetrating, or fusing.

The term “analyte” as used herein is a broad term, and is to be given its ordinary and customary meaning to a person of ordinary skill in the art (and is not to be limited to a special or customized meaning), and refers without limitation to a substance or chemical constituent in a biological fluid (e.g., blood, interstitial fluid, cerebral spinal fluid, lymph fluid, urine, sweat, saliva, etc.) that can be analyzed. Analytes can include naturally occurring substances, artificial substances, metabolites, and/or reaction products. In some examples, the analyte measured by the sensing regions, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); bilirubin, biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine; creatine kinase; creatine kinase MM isoenzyme; creatinine; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycerol; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; beta-hydroxybutyrate; ketones; lactate; lead; lipoproteins ((a), B/A-1, β); lysozyme; mefloquine; netilmicin; oxygen; phenobarbitone; phenytoin; phytanic/pristanic acid; potassium, sodium, and/or other blood electrolytes; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatic virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uric acid; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain examples. The analyte can be naturally present in the biological fluid, or endogenous, for example, a metabolic product, a hormone, an antigen, an antibody, and the like. Alternately, the analyte can be introduced into the body, or exogenous, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, RlTALlN®, CYLERT®, PRELUDIN®, DIDREX®, PRESTATE®, VORANIL®, SANDREX®, PLEGINE®; depressants (barbiturates, methaqualone, tranquilizers such as VALIUM®, LIBRIUM®, MILTOWN®, SERAX®, EQUANIL®, TRANXENE®; hallucinogens (phencyclidine, lysergic acid, mescaline, peyote, psilocybin); narcotics (heroin, codeine, morphine, opium, meperidine, PERCOCET®, PERCODAN®, TUSSIONEX®, fentanyl, DARVON®, TALWIN®, LOMOTIL®; designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid)(DOPAC®, homovanillic acid (HVA), 5-hydroxytryptamine (5HT), 5-hydroxyindoleacetic acid (FHIAA), and histamine.

The phrases “analyte-measuring device,” “analyte-monitoring device,” “analyte-sensing device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte or combination of analytes. For example, these phrases may refer without limitation to an instrument responsible for detection of a particular analyte or combination of analytes. In one example, the instrument includes a sensor coupled to circuitry disposed within a housing, and configure to process signals associated with analyte concentrations into information. In one example, such apparatuses and/or systems are capable of providing specific quantitative, semi-quantitative, qualitative, and/or semi qualitative analytical information using a biological recognition element combined with a transducing (detecting) element.

The term “amphiphilic” as used herein is a broad term and is to be given its ordinary and customary meaning to a person of ordinary skill in the art (and are not to be limited to a special or customized meaning), and refer without limitation to a chemical compound or polymer possessing both hydrophilic and hydrophobic segments or properties.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, and/or multi-analyte sensor device responsible for the detection of, or transduction of a signal associated with, a particular analyte or combination of analytes. In one example, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In one example, such biosensors and/or sensors are capable of providing specific quantitative, semi-quantitative, qualitative, semi qualitative analytical signals using a biological recognition element combined with a transducing (detecting) element.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms can provide specific quantitative, semi-quantitative, qualitative, semi qualitative analytical signals using a biological recognition element combined with a transducing (detecting) element.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The phrase “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “adhere” and “attach” as used herein are broad terms, and are to be given their ordinary and customary meaning to a person of ordinary skill in the art (and are not to be limited to a special or customized meaning), and refer without limitation to hold, bind, or stick, for example, by gluing, bonding, grasping, interpenetrating, or fusing.

The phrase “barrier cell layer” as used herein is a broad phrase, and is to be given its ordinary and customary meaning to a person of ordinary skill in the art (and is not to be limited to a special or customized meaning), and refers without limitation to a part of a foreign body response that forms a cohesive monolayer of cells (for example, macrophages and foreign body giant cells) that substantially block the transport of molecules and other substances to the implantable device.

The term “bioactive agent” as used herein is a broad term, and is to be given its ordinary and customary meaning to a person of ordinary skill in the art (and is not to be limited to a special or customized meaning), and refers without limitation to any substance that has an effect on or elicits a response from living tissue.

The phrases “biointerface membrane” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between host tissue and an implantable device. The terms “biointerface” and “bioprotective” are used interchangeably herein.

The term “biostable” as used herein is a broad term, and is to be given its ordinary and customary meaning to a person of ordinary skill in the art (and is not to be limited to a special or customized meaning), and refers without limitation to materials that are relatively resistant to degradation by processes that are encountered in vivo.

The phrase “cell processes” as used herein is a broad phrase, and is to be given its ordinary and customary meaning to a person of ordinary skill in the art (and is not to be limited to a special or customized meaning), and refers without limitation to pseudopodia of a cell.

The phrase “cellular attachment” as used herein is a broad phrase, and is to be given its ordinary and customary meaning to a person of ordinary skill in the art (and is not to be limited to a special or customized meaning), and refers without limitation to adhesion of cells and/or cell processes to a material at the molecular level, and/or attachment of cells and/or cell processes to microporous material surfaces or macroporous material surfaces. One example of a material used in the prior art that encourages cellular attachment to its porous surfaces is the BIOPORE™ cell culture support marketed by Millipore (Bedford, Mass.), and as described in Brauker et al., U.S. Pat. No. 5,741,330.

The term “cofactor” as used herein is a broad term, and is to be given its ordinary and customary meaning to a person of ordinary skill in the art (and is not to be limited to a special or customized meaning), and refers without limitation to one or more substances whose presence contributes to or is required for analyte-related activity of an enzyme. Analyte-related activity can include, but is not limited to, any one of or a combination of binding, electron transfer, and chemical transformation. Cofactors are inclusive of coenzymes, non-protein chemical compounds, metal ions and/or metal organic complexes. Coenzymes are inclusive of prosthetic groups and co-substrates.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrases “continuous analyte sensing” and “continuous multi-analyte sensing” as used herein are broad phrases, and are to be given their ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every second or less to about one week or more. In further examples, monitoring of analyte concentration is performed from about every 2, 3, 5, 7, 10, 15, 20, 25, 30, 35, 40, 45, 50, 55, or 60 seconds to about every 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In further examples, monitoring of analyte concentration is performed from about 10, 20, 30, 40 or 50 minutes to about every 1, 2, 3, 4, 5, 6, 7 or 8 hours. In further examples, monitoring of analyte concentration is performed from about every 8 hours to about every 12, 16, 20, or 24 hours. In further examples, monitoring of analyte concentration is performed from about every day to about every 1.5, 2, 3, 4, 5, 6, or 7 days. In further examples, monitoring of analyte concentration is performed from about every week to about every 1.5, 2, 3 or more weeks.

The term “coaxial” as used herein is to be construed broadly to include sensor architectures having elements aligned along a shared axis around a core that can be configured to have a circular, elliptical, triangular, polygonal, or other cross-section such elements can include electrodes, insulating layers, or other elements that can be positioned circumferentially around the core layer, such as a core electrode or core polymer wire.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. For example, an element is “coupled” if the element is covalently, communicatively, electrostatically, thermally connected, mechanically connected, magnetically connected, or ionically associated with, or physically entrapped, adsorbed to or absorbed by another element. Similarly, the phrases “operably connected”, “operably linked”, and “operably coupled” as used herein may refer to one or more components linked to another component(s) in a manner that facilitates transmission of at least one signal between the components. In some examples, components are part of the same structure and/or integral with one another as in covalently, electrostatically, mechanically, thermally, magnetically, ionically associated with, or physically entrapped, or absorbed (i.e. “directly coupled” as in no intervening element(s)). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein may refer to two or more system elements or components that are configured to be or have been electrically, mechanically, thermally, operably, chemically, or otherwise attached and detached without damaging any of the coupled elements or components. The phrase “permanently coupled” as used herein may refer to two or more system elements or components that are configured to be or have been electrically, mechanically, thermally, operably, chemically, or otherwise attached but cannot be uncoupled without damaging at least one of the coupled elements or components. covalently, electrostatically, ionically associated with, or physically entrapped, or absorbed

The phrase “defined edges” as used herein is a broad phrase, and is to be given its ordinary and customary meaning to a person of ordinary skill in the art (and is not to be limited to a special or customized meaning), and refers without limitation to abrupt, distinct edges or borders among layers, domains, coatings, or portions. “Defined edges” are in contrast to a gradual transition between layers, domains, coatings, or portions.

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of a membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “drift” as used herein is a broad term, and is to be given its ordinary and customary meaning to a person of ordinary skill in the art (and is not to be limited to a special or customized meaning), and refers without limitation to a progressive increase or decrease in signal over time that is unrelated to changes in host systemic analyte concentrations (e.g., host postprandial glucose concentrations). While not wishing to be bound by theory, it is believed that drift may be the result of a local decrease in glucose transport to the sensor, for example, due to formation of a foreign body capsule (FBC), or due to an insufficient amount of interstitial fluid surrounding the sensor, which results in reduced oxygen and/or glucose transport to the sensor. In one example, an increase in local interstitial fluid may slow or reduce drift and thus improve sensor performance. Drift may also be the result of sensor electronics, or algorithmic models used to compensate for noise or other anomalies that can occur with electrical signals in, for example, the picoAmp range, the femtoAmp range, the nanoAmp range, the microAmp range, the milliAmp range, the Amp range, etc.

The phrases “drug releasing membrane” and “drug releasing layer” as used interchangeably herein are each a broad phrase, and each are to be given its ordinary and customary meaning to a person of ordinary skill in the art (and is not to be limited to a special or customized meaning), and refers without limitation to a permeable or semi-permeable membrane which is permeable to one or more bioactive agents. In one example, the “drug releasing membrane” and “drug releasing layer” is typically of a few microns thickness or more and can be comprised of two or more domains. In one example the drug releasing layer and/or drug releasing membrane are substantially the same as the biointerface layer and/or biointerface membrane. In another example, the drug releasing layer and/or drug releasing membrane are distinct from the biointerface layer and/or biointerface membrane.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In one example this reaction is faradaic and results in charge transfer between the surface and its environment. In one example, hydrogen peroxide produced by an enzyme-catalyzed reaction of an analyte being oxidized on the surface results in a measurable electronic current. For example, in the detection of glucose, glucose oxidase produces hydrogen peroxide (H₂O₂) as a byproduct. The H₂O₂ reacts with the surface of the working electrode to produce two protons (2H⁺), two electrons (2e⁻) and one molecule of oxygen (O₂), which produces the electronic current being detected. In a counter electrode, a reducible species, for example, O₂ is reduced at the electrode surface so as to balance the current generated by the working electrode.

The term “electrolysis” as used herein is a broad term, and is to be given its ordinary and customary meaning to a person of ordinary skill in the art (and is not to be limited to a special or customized meeting), and refers without limitation to electrooxidation or electroreduction (collectively, “redox”) of a compound, either directly or indirectly, by one or more enzymes, cofactors, or mediators.

The phrase “hard segment” as used herein is a broad phrase, and is to be given its ordinary and customary meaning to a person of ordinary skill in the art (and is not to be limited to a special or customized meaning), and refers without limitation to an element of a copolymer, for example, a polyurethane, a polycarbonate polyurethane, or a polyurethane urea copolymer, which imparts resistance properties, e.g., resistance to bending or twisting. The phrase “hard segment” can be further characterized as a crystalline, semi-crystalline, or glassy material with a glass transition temperature determined by dynamic scanning calorimetry (“Tg”) typically above ambient temperature. Exemplary hard segment elements used to prepare a polycarbonate polyurethane, or a polyurethane urea hard segment include norbornane diisocyanate (NBDI), isophorone diisocynate (IPDI), tolylene diisocynate (TDI), 1,3-phenylene diisocyanate (MPDI), trans-1,3-bis(isocynatomethyl) cyclohexane (1,3-H6XDI), bicyclohexylmethane-4,4′-diisocynate (HMDI), 4,4′-Diphenylmethane diisocynate (MDI), trans-1,4-bis(isocynatomethyl) cyclohexane (1,4-H6XDI), 1,4-cyclohexyl diisocynate (CHDI), 1,4-phenylene diisocynate (PPDI), 3,3′-Dimethyl-4,4′-biphenyldiisocyanate (TODD, 1,6-hexamethylene diisocyanate (HDI), or combinations thereof.

The term “host” as used herein is a broad term, and is to be given its ordinary and customary meaning to a person of ordinary skill in the art (and is not to be limited to a special or customized meaning), and refers without limitation to mammals, for example humans.

The terms “indwelling,” “in dwelling,” “implanted,” or “implantable” as used herein are broad terms, and are to be given their ordinary and customary meaning to a person of ordinary skill in the art (and are not to be limited to a special or customized meaning), and refer without limitation to objects including sensors that are inserted, or configured to be inserted, subcutaneously (i.e. in the layer of fat between the skin and the muscle), intracutaneously (i.e. penetrating the stratum corneum and positioning within the epidermal or dermal strata of the skin), or transcutaneously (i.e. penetrating, entering, or passing through intact skin), which may result in a sensor that has an in vivo portion and an ex vivo portion. The term “indwelling” also encompasses an object which is configured to be inserted subcutaneously, intracutaneously, or transcutaneously, whether or not it has been inserted as such.

The phrase “insertable surface area” as used herein is a broad phrase, and is to be given its ordinary and customary meaning to a person of ordinary skill in the art (and is not to be limited to a special or customized meaning), and refers without limitation to a surface area of an insertable portion of an analyte sensor including, but not limited to, the geometric surface area e.g., planar, flat or substantially planar, and/or coaxial utilized substrates in the analyte sensor as described herein.

The phrase “insertable volume” as used herein is a broad phrase, and is to be given its ordinary and customary meaning to a person of ordinary skill in the art (and is not to be limited to a special or customized meaning), and refers without limitation to a volume ahead of and alongside a path of insertion of an insertable portion of an analyte sensor, as described herein, as well as an incision made in the skin to insert the insertable portion of the analyte sensor. The insertable volume also includes up to 5 mm radially or perpendicular to the volume ahead of and alongside the path of insertion.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In one example of an electrochemical sensor, interfering species are compounds which produce a signal that is not analyte-specific due to a reaction on an electrochemically active surface.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

As used herein, the terms “machine-storage medium,” “device-storage medium,” “computer-storage medium” (referred to collectively as “machine-storage medium” mean the same thing and may be used interchangeably in this disclosure. The terms refer to a single or multiple storage devices and/or media (e.g., a centralized or distributed database, and/or associated caches and servers) that store executable instructions and/or data, as well as cloud-based storage systems or storage networks that include multiple storage apparatus or devices. The terms shall accordingly be taken to include, but not be limited to, solid-state memories, and optical and magnetic media, including memory internal or external to processors. Specific examples of machine-storage media, computer-storage media, and/or device-storage media include non-volatile memory, including by way of example semiconductor memory devices, e.g., erasable programmable read-only memory (EPROM), electrically erasable programmable devices; magnetic disks such as internal hard disks and removable disks; magneto-optical disks; and CD-ROM and DVD-ROM disks. The terms machine-storage media, computer-storage media, and device-storage media specifically exclude carrier waves, modulated data signals, and other such media, at least some of which are covered under the term “signal medium” discussed below.

The term and phrase “mediator” and “redox mediator” as used herein are broad terms and phrases, and are to be given their ordinary and customary meaning to a person of ordinary skill in the art (and is not to be limited to a special or customized meaning), and refers without limitation to any chemical compound or collection of compounds capable of electron transfer, either directly, or indirectly, between an analyte, analyte precursor, analyte surrogate, analyte-reduced or analyte-oxidized enzyme, or cofactor, and an electrode surface held at a potential. In one example the mediator accepts electrons from, or transfer electrons to, one or more enzymes or cofactors, and/or exchanges electrons with the sensor system electrodes. In one example, mediators are transition-metal coordinated organic molecules which are capable of reversible oxidation and reduction reactions. In other examples, mediators may be organic molecules or metals which are capable of reversible oxidation and reduction reactions.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst (e.g., one or more enzymes) for enabling an enzymatic reaction, limitation or blocking of interfering species, provision of hydrophilicity at the electrochemically reactive surfaces of the sensor interface, service as an interface between host tissue and the implantable device, modulation of host tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to oxygen and is optionally permeable to, e.g., glucose or another analyte. In one example, the membrane system comprises an enzyme, which enables an analyte reaction to occur whereby a concentration of the analyte can be measured.

The phrases “machine-readable medium,” ‘computer-readable medium” and ‘device-readable medium” mean the same thing and may be used interchangeably in this disclosure. The phrases are inclusive of both machine-storage media and signal media operably coupled to a sensor, biosensor, analyte sensing device, or analyte monitoring device. Thus, the phrases are inclusive of both storage devices/media and carrier waves/modulated data signals operably coupled to a sensor, biosensor, analyte sensing device, or analyte monitoring device.

The term “micro,” as used herein is a broad term, and is to be given its ordinary and customary meaning to a person of ordinary skill in the art (and is not to be limited to a special or customized meaning), and refers without limitation to a small object or scale of approximately 10⁻⁶ m that is not visible without magnification. The term “micro” is in contrast to the term “macro,” which refers to a large object that may be visible without magnification. Similarly, the term “nano” refers to a small object or scale of approximately 10⁻⁹ m.

The term “noise,” as used herein, is a broad term and is used in its ordinary sense, including, without limitation, a signal detected by the sensor or sensor electronics that is unrelated to analyte concentration and can result in reduced sensor performance. One type of noise has been observed during the few hours (e.g., about 2 to about 24 hours) after sensor insertion. After the first 24 hours, the noise may disappear or diminish, but in some hosts, the noise may last for about three to four days. In some cases, noise can be reduced using predictive modeling, artificial intelligence, and/or algorithmic means. In other cases, noise can be reduced by addressing immune response factors associated with the presence of the implanted sensor, such as using a drug releasing layer with at least one bioactive agent. For example, noise of one or more exemplary biosensors as presently disclosed can be determined and then compared qualitatively or quantitatively. By way of example, by obtaining a raw signal timeseries with a fixed sampling interval (in units of pA), a smoothed version of the raw signal timeseries can be obtained, e.g., by applying a 3rd order lowpass digital Chebyshev Type II filter. Others smoothing algorithms can be used. At each sampling interval, an absolute difference, in units of pA, can be calculated to provide a smoothed timeseries. This smoothed timeseries can be converted into units of mg/dL, (the unit of “noise”), using a glucose sensitivity timeseries, in units of pA/mg/dL, where the glucose sensitivity timeseries is derived by using a mathematical model between the raw signal and reference blood glucose measurements (e.g., obtained from Blood Glucose Meter). Optionally, the timeseries can be aggregated as desired, e.g., by hour or day. Comparison of corresponding timeseries between different exemplary biosensors with the presently disclosed drug releasing layer and one or more bioactive agents provides for qualitative or quantitative determination of improvement of noise.

The term “optional” or “optionally” as used herein is a broad term, and is to be given its ordinary and customary meaning to a person of ordinary skill in the art (and is not to be limited to a special or customized meaning), and, without limitation, means that the subsequently described event or circumstance can or cannot occur, and that the description includes instances where the event or circumstance occurs and instances where it does not.

The term “planar” as used herein is to be interpreted broadly to describe sensor architecture having a substrate including at least a first surface and an opposing second surface, and for example, comprising a plurality of elements arranged on one or more surfaces or edges of the substrate. The plurality of elements can include conductive or insulating layers or elements configured to operate as a circuit. The plurality of elements may or may not be electrically or otherwise coupled. In one example, planar includes one or more edges separating the opposed surfaces.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme domain or layer. If the sensor is deemed to be the point of reference and the enzyme domain is positioned nearer to the sensor than the biointerface layer, then the enzyme domain is more proximal to the sensor than the biointerface layer.

The phrase and term “processor module” and “microprocessor” as used herein are each a broad phrase and term, and are to be given their ordinary and customary meaning to a person of ordinary skill in the art (and are not to be limited to a special or customized meaning), and refer without limitation to a computer system, state machine, processor, or the like designed to perform arithmetic or logic operations using logic circuitry that responds to and processes the basic instructions that drive a computer.

The term “semi-continuous” as used herein is a broad term, and is to be given its ordinary and customary meaning to a person of ordinary skill in the art (and is not to be limited to a special or customized meaning), and refers without limitation to a portion, coating, domain, or layer that includes one or more continuous and noncontinuous portions, coatings, domains, or layers. For example, a coating disposed around a sensing region but not about the sensing region is “semi-continuous.”

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms are capable of providing specific quantitative, semi-quantitative, qualitative, semi qualitative analytical signals using a biological recognition element combined with a transducing and/or detecting element.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an enzyme, for example, glucose oxidase, DNA, RNA, or a protein or aptamer, for example, one or more periplasmic binding protein (PBP) or mutant or fusion protein thereof having one or more analyte binding regions, each region capable of specifically or reversibly binding to and/or reacting with at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level, for example, glucose, ketone, lactate, potassium, etc., in the biological sample.

In one example, the sensing region or sensing portion can comprise at least a portion of a conductive substrate or at least a portion of a conductive surface, for example, a wire (coaxial) or conductive trace or a substantially planar substrate including substantially planar trace(s), and a membrane. In one example, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface. In some examples, the sensing membrane further comprises an enzyme domain, for example, an enzyme domain, and an electrolyte phase, for example, a free-flowing liquid phase comprising an electrolyte-containing fluid described further below. The terms are broad enough to include the entire device, or only the sensing portion thereof (or something in between).

In another example, the sensing region can comprise one or more periplasmic binding protein (PBP) including mutant or fusion protein thereof, or aptamers having one or more analyte binding regions, each region capable of specifically and reversibly binding to at least one analyte. Alterations of the aptamer or mutations of the PBP can contribute to or alter one or more of the binding constants, long-term stability of the protein, including thermal stability, to bind the protein to a special encapsulation matrix, membrane or polymer, or to attach a detectable reporter group or “label” to indicate a change in the binding region or transduce a signal corresponding to the one or more analytes present in the biological fluid. Specific examples of changes in the binding region include, but are not limited to, hydrophobic/hydrophilic environmental changes, three-dimensional conformational changes, changes in the orientation of amino/nucleic acid side chains in the binding region of proteins, and redox states of the binding region. Such changes to the binding region provide for transduction of a detectable signal corresponding to the one or more analytes present in the biological fluid.

In one example, the sensing region determines the selectivity among one or more analytes, so that only the analyte which has to be measured leads to (transduces) a detectable signal. The selection may be based on any chemical or physical recognition of the analyte by the sensing region, where the chemical composition of the analyte is unchanged, or in which the sensing region causes or catalyzes a reaction of the analyte that changes the chemical composition of the analyte.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, in one example, a sensor has a sensitivity (or slope) of from about 1 to about 100 picoAmps of current for every 1 mg/dL of analyte.

The phrases “signal medium” or “transmission medium” shall be taken to include any form of modulated data signal, carrier wave, and so forth. The phrase “modulated data signal” means a signal that has one or more of its characteristics set or changed in such a matter as to encode information in the signal.

The phrases and terms “small diameter sensor,” “small structured sensor,” and “micro-sensor” as used herein are broad phrases and terms, and are to be given their ordinary and customary meaning to a person of ordinary skill in the art (and are not to be limited to a special or customized meaning), and refer without limitation to sensing mechanisms that are less than about 2 mm in at least one dimension. In further examples, the sensing mechanisms are less than about 1 mm in at least one dimension. In some examples, the sensing mechanism (sensor) is less than about 0.95, 0.9, 0.85, 0.8, 0.75, 0.7, 0.65, 0.6, 0.5, 0.4, 0.3, 0.2, or 0.1 mm. In some examples, the maximum dimension of an independently measured length, width, diameter, thickness, or circumference of the sensing mechanism does not exceed about 2 mm. In some examples, the sensing mechanism is a coaxial sensor, wherein the diameter of the sensor is less than about 1 mm, see, for example, U.S. Pat. No. 6,613,379 to Ward et al. and U.S. Pat. No. 7,497,827 to Brister et al., both of which are incorporated herein by reference in their entirety. In some alternate examples, the sensing mechanism includes electrodes deposited on a planar or substantially planar substrate, wherein the thickness of the implantable portion is less than about 1 mm, see, for example U.S. Pat. No. 6,175,752 to Say et al. and U.S. Pat. No. 5,779,665 to Mastrototaro et. al., both of which are incorporated herein by reference in their entirety.

The phrase “soft segment” as used herein is a broad phrase, and is to be given its ordinary and customary meaning to a person of ordinary skill in the art (and is not to be limited to a special or customized meaning), and refers without limitation to an element of a copolymer, for example, a polyurethane, a polycarbonate-polyurethane, or a polyurethane urea copolymer, which imparts flexibility to the chain. The phrase “soft segment” can be further characterized as an amorphous material with a low Tg, e.g., a Tg not typically higher than ambient temperature or normal mammalian body temperature.

The phrase “solid portions” as used herein is a broad term, and is to be given its ordinary and customary meaning to a person of ordinary skill in the art (and is not to be limited to a special or customized meaning), and refers without limitation to portions of a membrane's material having a mechanical structure that demarcates cavities, voids, or other non-solid portions.

The terms “transducing” or “transduction” and their grammatical equivalents as are used herein are broad terms, and are to be given their ordinary and customary meaning to a person of ordinary skill in the art (and is not to be limited to a special or customized meaning), and refer without limitation to optical, electrical, electrochemical, acoustical/mechanical, or colorimetrical technologies and methods. Electrochemical properties include current and/or voltage, inductance, capacitance, impedance, transconductance, and potential. Optical properties include absorbance, fluorescence/phosphorescence, fluorescence/phosphorescence decay rate, wavelength shift, dual wave phase modulation, bio/chemiluminescence, reflectance, light scattering, and refractive index. For example, the sensing region transduces the recognition of analytes into a semi-quantitative or quantitative signal.

As used herein, the phrase “transducing element” as used herein is a broad phrase, and are to be given their ordinary and customary meaning to a person of ordinary skill in the art (and is not to be limited to a special or customized meaning), and refers without limitation to analyte recognition moieties capable of facilitating, directly or indirectly, with detectable signal transduction corresponding to the presence and/or concentration of the recognized analyte. In one example, a transducing element is one or more enzymes, one or more aptamers, one or more ionophores, one or more capture antibodies, one or more proteins, one or more biological cells, one or more oligonucleotides, and/or one or more DNA or RNA moieties. Transcutaneous continuous multi-analyte sensors can be used in vivo over various lengths of time. The continuous multi-analyte sensor systems discussed herein can be transcutaneous devices, in that a portion of the device may be inserted through the host's skin and into the underlying soft tissue while a portion of the device remains on the surface of the host's skin. In one aspect, in order to overcome the problems associated with noise or other sensor function in the short-term, one example employs materials that promote formation of a fluid pocket around the sensor, for example architectures such as a porous biointerface membrane or matrices that create a space between the sensor and the surrounding tissue. In some examples, a sensor is provided with a spacer adapted to provide a fluid pocket between the sensor and the host's tissue. It is believed that this spacer, for example a biointerface material, matrix, structure, and the like as described in more detail elsewhere herein, provides for oxygen and/or glucose transport to the sensor.

### Membrane Systems

Membrane systems disclosed herein are suitable for use with implantable devices in contact with a biological fluid. For example, the membrane systems can be utilized with implantable devices, such as devices for monitoring and determining analyte levels in a biological fluid, for example, devices for monitoring glucose levels for individuals having diabetes. In some examples, the analyte-measuring device is a continuous device. The analyte-measuring device can employ any suitable sensing element to provide the raw signal, including but not limited to those involving enzymatic, chemical, physical, electrochemical, spectrophotometric, amperometric, potentiometric, polarimetric, calorimetric, radiometric, immunochemical, or like elements.

Suitable membrane systems for the aforementioned multi-analyte systems and devices can include, for example, membrane systems disclosed in U.S. Pat. Nos. 6,015,572, 5,964,745, and 6,083,523, which are incorporated herein by reference in their entireties for their teachings of membrane systems.

In general, the membrane system includes a plurality of domains, for example, an electrode domain, an interference domain, an enzyme domain, a resistance domain, and a biointerface domain. The membrane system can be deposited on the exposed electroactive surfaces using known thin film techniques (for example, vapor deposition, spraying, electrodepositing, dipping, brush coating, film coating, drop-let coating, and the like). Additional steps may be applied following the membrane material deposition, for example, drying, annealing, and curing (for example, UV curing, thermal curing, moisture curing, radiation curing, and the like) to enhance certain properties such as mechanical properties, signal stability, and selectivity. In a typical process, upon deposition of the resistance domain membrane, a biointerface/drug releasing layer having a “dry film” thickness of from about 0.05 micron (μm), or less, to about 1, 2, 3, 4, 5, 6, 7, 8, 9, 10, 11, 12, 13, 14, 15, or 16 μm is formed. “Dry film” thickness refers to the thickness of a cured film cast from a coating formulation by standard coating techniques.

In certain examples, the biointerface/drug releasing layer is formed of a biointerface polymer, wherein the biointerface polymer comprises one or more membrane domains comprising polyurethane and/or polyurea segments and one or more zwitterionic repeating units. In some examples, the biointerface/drug releasing layer coatings are formed of a polyurethane urea having carboxyl betaine groups incorporated in the polymer and non-ionic hydrophilic polyethylene oxide segments, wherein the polyurethane urea polymer is dissolved in organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the bioprotective polymers are formed of a polyurethane urea having carboxylic acid groups and carboxyl betaine groups incorporated in the polymer and non-ionic hydrophilic polyethylene oxide segments, wherein the polyurethane urea polymer is dissolved in an organic or non-organic solvent system in a coating formulation, and is crosslinked with an a carbodiimide (e.g., 1-ethyl-3-(3-dimethylaminopropyl)carbodiimide (EDC) or polycarbodiimide crosslinkers) and cured at a moderate temperature of about 50° C. In one example, polycarbodiimide crosslinkers are used.

In other examples, the biointerface/drug releasing layer coatings are formed of a polyurethane urea having sulfobetaine groups incorporated in the polymer and non-ionic hydrophilic polyethylene oxide segments, wherein the polyurethane urea polymer is dissolved in an organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the biointerface polymers are formed of a polyurethane urea having unsaturated hydrocarbon groups and sulfobetaine groups incorporated in the polymer and non-ionic hydrophilic polyethylene oxide segments, wherein the polyurethane urea polymer is dissolved in an organic or non-organic solvent system in a coating formulation, and is crosslinked in the presence of initiators with heat or irradiation including UV, LED light, electron beam, and the like, and cured at a moderate temperature of about 50° C. Examples of unsaturated hydrocarbon includes allyl groups, vinyl groups, acrylate, methacrylate, alkenes, alkynes, and the like.

In some examples, tethers are used. A tether is a polymer or chemical moiety which does not participate in the (electro) chemical reactions involved in sensing, but forms chemical bonds with the (electro) chemically active components of the membrane. In some examples these bonds are covalent. In one example, a tether may be formed in solution prior to one or more interlayers of a membrane being formed, where the tether bonds two (electro) chemically active components directly to one another or alternately, the tether(s) bond (electro) chemically active component(s) to polymeric backbone structures. In another example, (electro) chemically active components are comixed along with crosslinker(s) with tunable lengths (and optionally polymers) and the tethering reaction occurs as in situ crosslinking. Tethering may be employed to maintain a predetermined number of degrees of freedom of NAD(P)H for effective enzyme catalysis, where “effective” enzyme catalysis causes the analyte sensor to continuously monitor one or more analytes for a period of from about 5 days to about 15 days or more.

### Membrane Fabrication

Polymers can be processed by solution-based techniques such as spraying, dipping, casting, electrospinning, vapor deposition, spin coating, coating, and the like. Water-based polymer emulsions can be fabricated to form membranes by methods similar to those used for solvent-based materials. In both cases the evaporation of a volatile liquid (e.g., organic solvent or water) leaves behind a film of the polymer. Cross-linking of the deposited film or layer can be performed through the use of multi-functional reactive ingredients by a number of methods. The liquid system can cure by heat, moisture, high-energy radiation, ultraviolet light, or by completing the reaction, which produces the final polymer in a mold or on a substrate to be coated.

In some examples, the wetting property of the membrane (and by extension the extent of sensor drift exhibited by the sensor) can be adjusted and/or controlled by creating covalent cross-links between surface-active group-containing polymers, functional-group containing polymers, polymers with zwitterionic groups (or precursors or derivatives thereof), and combinations thereof. Cross-linking can have a substantial effect on film structure, which in turn can affect the film's surface wetting properties. Crosslinking can also affect the film's tensile strength, mechanical strength, water absorption rate and other properties.

Cross-linked polymers can have different cross-linking densities. In certain examples, cross-linkers are used to promote cross-linking between layers. In other examples, in replacement of (or in addition to) the cross-linking techniques described above, heat is used to form cross-linking. For example, in some examples, imide and amide bonds can be formed between two polymers as a result of high temperature. In some examples, photo cross-linking is performed to form covalent bonds between the polycationic layers(s) and polyanionic layer(s). One major advantage to photo-cross-linking is that it offers the possibility of patterning. In certain examples, patterning using photo-cross linking is performed to modify the film structure and thus to adjust the wetting property of the membranes and membrane systems, as discussed herein.

Polymers with domains or segments that are functionalized to permit cross-linking can be made by methods at least as discussed herein. For example, polyurethaneurea polymers with aromatic or aliphatic segments having electrophilic functional groups (e.g., carbonyl, aldehyde, anhydride, ester, amide, isocyano, epoxy, allyl, or halo groups) can be crosslinked with a crosslinking agent that has multiple nucleophilic groups (e.g., hydroxyl, amine, urea, urethane, or thiol groups). In further examples, polyurethaneurea polymers having aromatic or aliphatic segments having nucleophilic functional groups can be crosslinked with a crosslinking agent that has multiple electrophilic groups. In one example, polycarbodiimide crosslinkers are used. Still further, polyurethaneurea polymers having hydrophilic segments having nucleophilic or electrophilic functional groups can be crosslinked with a crosslinking agent that has multiple electrophilic or nucleophilic groups. Unsaturated functional groups on the polyurethane urea can also be used for crosslinking by reacting with multivalent free radical agents. Non-limiting examples of suitable cross-linking agents include isocyanate, carbodiimide, glutaraldehyde, aziridine, silane, or other aldehydes, epoxy, acrylates, free-radical based agents, ethylene glycol diglycidyl ether (EGDE), poly(ethylene glycol) diglycidyl ether (PEG-DE), or dicumyl peroxide (DCP). In one example, from about 0.1% to about 15% w/w of cross-linking agent is added relative to the total dry weights of cross-linking agent and polymers added when blending the ingredients. In another example, about 1% to about 10% w/w of cross-linking agent is added relative to the total dry weights of cross-linking agent and polymers added when blending the ingredients. In yet another example, about 5% to about 15% w/w of cross-linking agent is added relative to the total dry weights of cross-linking agent and polymers added when blending the ingredients. During the curing process, substantially all of the cross-linking agent is believed to react, leaving substantially no detectable unreacted cross-linking agent in the final film.

Polymers disclosed herein can be formulated into mixtures that can be drawn into a film or applied to a surface using methods such as spraying, self-assembling monolayers (SAMs), painting, dip-coating, vapor depositing, molding, 3-D printing, slot die coating, pico jet printing, piezo inkjet printing, lithographic techniques (e.g., photolithograph), micro- and nano-pipetting printing techniques, silk-screen printing, etc.). The mixture can then be cured under high temperature (e.g., from about 30° C. to about 150° C.). Other suitable curing methods can include ultraviolet, e-beam, or gamma radiation, for example.

In some circumstances, using continuous multianalyte monitoring systems including sensor(s) configured with bioprotective and/or drug releasing membranes, it is believed that that foreign body response is the dominant event surrounding extended implantation of an implanted device and can be managed or manipulated to support rather than hinder or block analyte transport. In another aspect, in order to extend the lifetime of the sensor, one example employs materials that promote vascularized tissue ingrowth, for example within a porous biointerface membrane. For example, tissue in-growth into a porous biointerface material surrounding a sensor may promote sensor function over extended periods of time (e.g., weeks, months, or years). It has been observed that in-growth and formation of a tissue bed can take up to 3 weeks. Tissue ingrowth and tissue bed formation is believed to be part of the foreign body response. As will be discussed herein, the foreign body response can be manipulated by the use of porous bioprotective materials that surround the sensor and promote ingrowth of tissue and microvasculature over time.

Accordingly, a sensor as discussed in examples herein may include a biointerface layer. The biointerface layer, like the drug releasing layer, may include, but is not limited to, for example, porous biointerface materials including a solid portion and interconnected cavities, all of which are described in more detail elsewhere herein. The biointerface layer can be employed to improve sensor function in the long term (e.g., after tissue ingrowth).

Accordingly, a sensor as discussed in examples herein may include a drug releasing membrane at least partially functioning as or in combination with a biointerface membrane. The drug releasing membrane may include, for example, materials including a hard-soft segment polymer with hydrophilic and optionally hydrophobic domains, all of which are described in more detail elsewhere herein, can be employed to improve sensor function in the long term (e.g., after tissue ingrowth). In one example, the materials including a hard-soft segment polymer with hydrophilic and optionally hydrophobic domains are configured to release a combination of a derivative form of dexamethasone or dexamethasone acetate with dexamethasone such that one or more different rates of release of the anti-inflammatory is achieved and the useful life of the sensor is extended. Other suitable drug releasing membranes of the present disclosure can be selected from silicone polymers, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), poly vinyl acetate, ethylene vinyl acetate (EVA), polybutylene terephthalate (PBT), polymethylmethacrylate (PMMA), polyether ether ketone (PEEK), polyamides, polyurethanes and copolymers and blends thereof, polyurethane urea polymers and copolymers and blends thereof, cellulosic polymers and copolymers and blends thereof, poly(ethylene oxide) and copolymers and blends thereof, poly(propylene oxide) and copolymers and blends thereof, polysulfones and block copolymers thereof including, for example, di-block, tri-block, alternating, random and graft copolymers cellulose, hydrogel polymers, poly(2-hydroxyethyl methacrylate, pHEMA) and copolymers and blends thereof, hydroxyethyl methacrylate, (HEMA) and copolymers and blends thereof, polyacrylonitrile-polyvinyl chloride (PAN-PVC) and copolymers and blends thereof, acrylic copolymers and copolymers and blends thereof, nylon and copolymers and blends thereof, polyvinyl difluoride, polyanhydrides, poly(l-lysine), poly(L-lactic acid), hydroxyethylmetharcrylate and copolymers and blends thereof, and hydroxyapeptite and copolymers and blends thereof.

### Sensing Mechanism

In general, the analyte sensors of the present disclosure include a sensing mechanism with a small structure (e.g., small structured-, micro- or small diameter sensor), for example, a coaxial or planar sensor, in at least a portion thereof. As used herein a “small structure” refers to an architecture with at least one dimension less than about 1 mm. The small-structured sensing mechanism can be coaxial-based, or substrate-based (flat or substantially planar substrate, that can be single or double-sided, which may or may include one or more sensor elements on any of the sides or surfaces), or any other architecture. In some alternate examples, the term “small structure” can also refer to slightly larger structures, such as those having their smallest dimension being greater than about 1 mm, however, the architecture (e.g., mass or size) is designed to minimize the foreign body response due to size and/or mass.

The present disclosure is inclusive of sensor systems including two or more sensors, each sensor being configured to sense a different analyte. The two or more sensors can be configured to function independently or simultaneously to sense two or more analytes concurrently, sequentially, and/or randomly (which is inclusive of events that can take place independently in picoseconds, nanoseconds, milliseconds, seconds, or minutes) or in an alternating or overlapping fashion. The two or more sensors of the sensor system can be communicatively coupled to electronics, e.g., a single transmitter or receiver. The two or more sensors of the sensor system can be communicatively coupled to separate, independent electronics.

In one example of a continuous analyte monitoring system, a first, single, sensor is configured to continuously monitor at least a first analyte (e.g., glycose, glycerol, lactate, bilirubin, oxygen, etc.) and a second, different, analyte. In this example, the single sensor may include a single coaxial or planar sensor configured to monitor the at least first analyte and the second analyte. In another example, a first sensor is configured to monitor the first analyte, and a second sensor is configured to continuously monitor a second analyte (e.g., ketones). Each of the first sensor and the second sensor may be planar, substantially planar, or coaxial, or a combination of two or more top, side, or cross-sectional geometries. In one example, each of the first sensor and the second sensor are communicatively coupled to the same sensor electronics and networking elements to continuously monitor and provide feedback to a device, e.g., a mobile device, tablet, laptop, wearable technology (clothing, jewelry, other accessory) or other IoT (internet-of-things) device or combinations of devices. In another example, the first sensor and the second sensor are communicatively coupled to independent sensor electronics and networking elements. Each of the first sensor and the second sensor are positioned in a subject in a subcutaneous layer through a skin layer. In another example, a sensor system is configured as a monolithic sensor body having both the first sensor and the second sensor with their electrodes configured to detect two or more analytes. At least one plurality of electrodes of the sensor system is configured to detect a first analyte, and a second plurality of electrodes is configured to detect a second analyte. The sensor system is positioned in a subject in a subcutaneous layer through a skin layer. In yet another example, a sensor system includes a first sensor and a second sensor, where each sensor of the sensor system includes one or more fiber elements. For example, two or more sensors such as the first sensor and the second sensor may be electrically, mechanically, or otherwise coupled together ex vivo, in vivo, or both. Each of the first sensor and the second sensor of the sensor system is positioned in a subject in a subcutaneous layer through a skin layer.

The multi-analyte sensor device and systems discussed herein may include elements such as on-body wearable devices, wireless communication capabilities, electronics, software, GUI(s), or other elements configured to cause the analyte monitoring systems to continuously monitor analyte levels in a host. Various alerts and actions may be taken in response to this monitoring. As discussed herein, an “on-body” device or wearable device includes devices configured to couple to a host for at least a predetermined period of time via one or more coupling elements including an in-vivo component such as a sensor, and/or adhesives, mechanical elements, electrical elements, magnetic elements, or other combinations of elements.

### Sensing Membrane

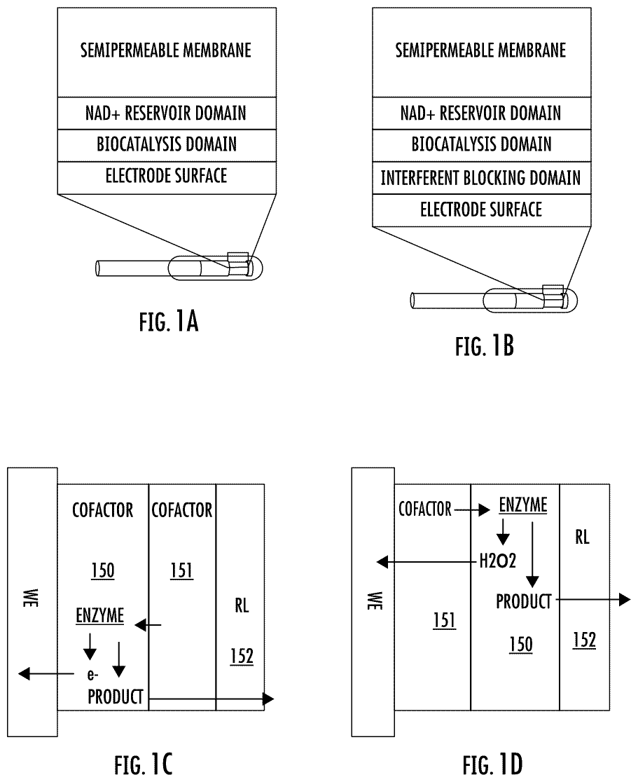

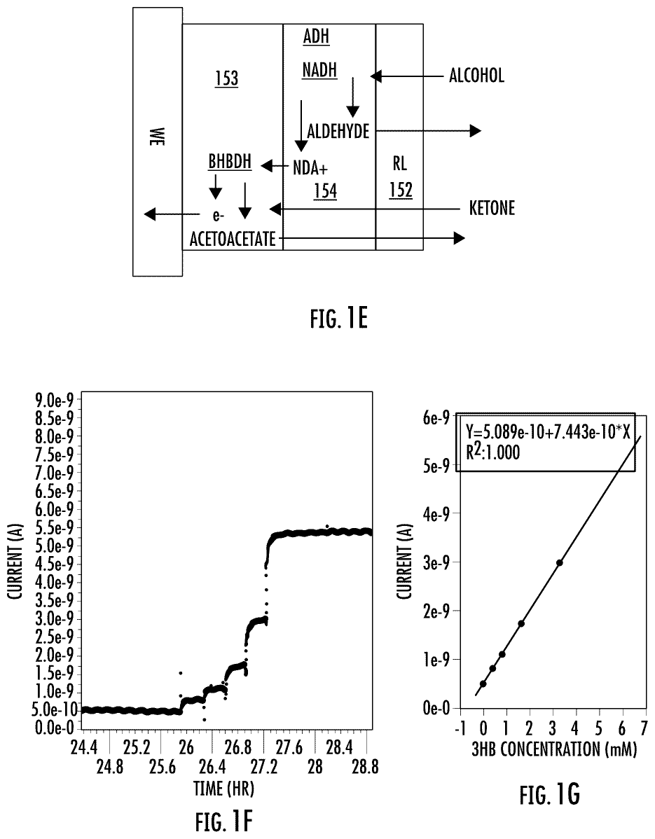

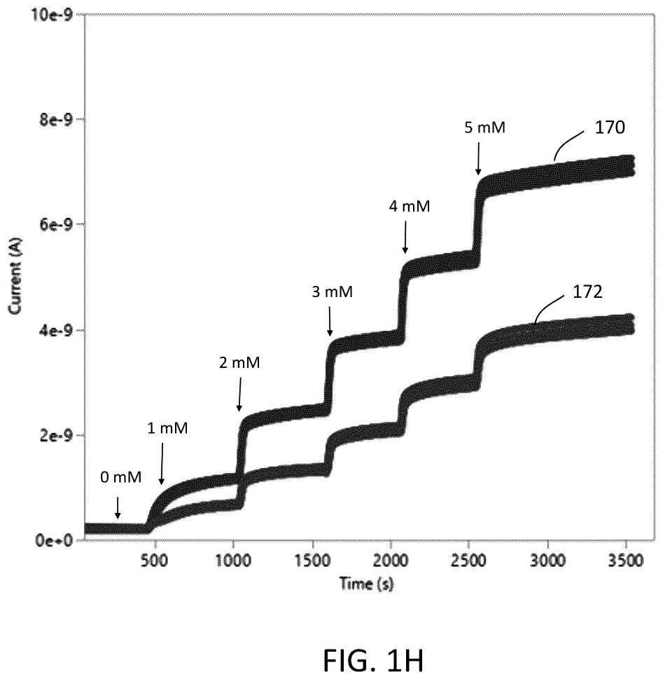

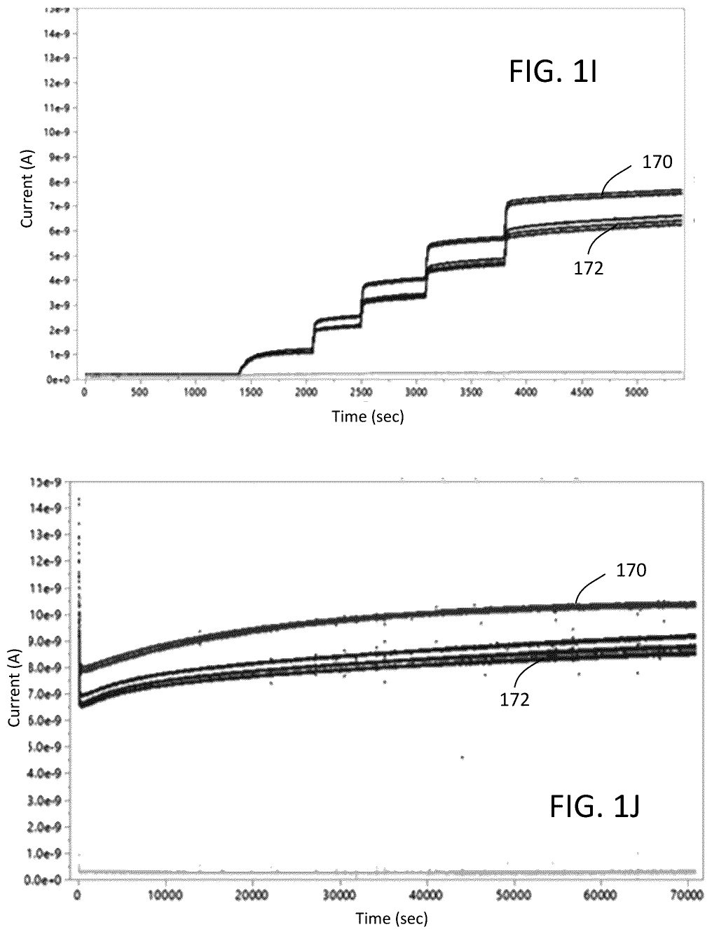

In some examples, as shown in FIG. 1, a sensing membrane is disposed over the electroactive surfaces of the continuous multi-analyte sensor 100 and includes one or more domains or layers. In general, the sensing membrane functions to control the flux of a biological fluid there through and/or to protect sensitive regions of the sensor from contamination by the biological fluid, for example. Some 1 electrochemical enzyme-based analyte sensors generally include a sensing membrane that controls the flux of the analyte being measured, protects the electrodes from contamination of the biological fluid, and/or provides an enzyme that catalyzes the reaction of the analyte with a co-factor, for example. See, e.g., U.S. Pat. Appl. Pub. No. 2005/0245799 to Brauker et al. and U.S. Pat. No. 7,497,827 to Brister et al., which are incorporated herein by reference in their entirety.

The sensing membranes of the present disclosure can include any membrane configuration suitable for use with any analyte sensor (such as described in more detail above). In general, the sensing membranes of the present disclosure include one or more domains, all or some of which can be adhered to or deposited on the analyte sensor as is appreciated by a person of ordinary skill in the art. In one example, the sensing membrane generally provides one or more of the following functions: 1) protection of the exposed electrode surface from the biological environment, 2) diffusion resistance (limitation) of the analyte, 3) a catalyst for enabling an enzymatic reaction, 4) limitation or blocking of interfering species, and 5) hydrophilicity at the electrochemically reactive surfaces of the sensor interface, such as described in U.S. Pat. No. 7,497,827 to Brister et al., referenced above. The sensing membranes discussed herein may include one or more adhesive layers positioned in between two adjacent membrane layers. In one example, the one or more adhesive layers can increase robustness and adherence, thus improving the sensing membrane integrity. In various examples, the adhesive layer may include silane groups, polyvinyl alcohol (PVA), glutaraldehyde, or silicone-based or silicone-including materials, or other adhesives or combinations of adhesives.

### Electrode Domain

In some examples, the membrane system comprises an optional electrode domain. The electrode domain is provided to promote and/or enhance an electrochemical reaction between the electroactive surfaces of the working electrode and the reference electrode, and thus the electrode domain is situated more proximal to the electroactive surfaces than the enzyme domain. In some examples, the electrode domain includes a semipermeable coating that maintains a layer of water at the electrochemically reactive surfaces of the sensor, for example, a humectant in a binder material can be employed as an electrode domain; this allows for the full transport of ions in the aqueous environment. The electrode domain can also assist in stabilizing the operation of the sensor by overcoming electrode start-up and drifting problems caused by inadequate electrolyte. The material that forms the electrode domain can also protect against pH-mediated damage that can result from the formation of a large pH gradient due to the electrochemical activity of the electrodes.

In one example, the electrode domain includes a flexible, water-swellable, hydrogel film having a “dry film” thickness of from about 0.05 micron or less to about 20 microns or more. In some examples, the “dry film” thickness is from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns. In further examples, the “dry film” thickness is from about 2, 2.5 or 3 microns to about 3.5, 4, 4.5, or 5 microns. “Dry film” thickness refers to the thickness of a cured film cast from a coating formulation by standard coating techniques.

In certain examples, the electrode domain is formed of a curable mixture of a urethane polymer and a hydrophilic polymer. Coatings are formed of a polyurethane polymer having carboxylate functional groups and non-ionic hydrophilic polyether segments, wherein the polyurethane polymer is crosslinked with a water soluble carbodiimide (e.g., 1-ethyl-3-(3-dimethylaminopropyl)carbodiimide (EDC) or polycarbodiimide crosslinker) in the presence of polyvinylpyrrolidone and cured at a moderate temperature of about 50° C.

In some examples, the electrode domain is deposited by spray or dip-coating the electroactive surfaces of the sensor. In further examples, the electrode domain is formed by dip-coating the electroactive surfaces in an electrode solution and curing the domain for a time of from about 15 to about 30 minutes at a temperature of from about 40 to about 55° C. (and can be accomplished under vacuum (e.g., 20 mmHg to 30 mmHg)). In examples wherein dip-coating is used to deposit the electrode domain, a insertion rate of from about 1 to about 3 inches per minute, with a dwell time of from about 0.5 to about 2 minutes, and a withdrawal rate of from about 0.25 to about 2 inches per minute provide a functional coating. However, values outside of those set forth above can be acceptable or even desirable in certain examples, for example, dependent upon viscosity and surface tension as is appreciated by a person of ordinary skill in the art. In one example, the electroactive surfaces of the electrode system are dip-coated one time (one layer) and cured at 50° C. under vacuum for 20 minutes.

As discussed herein, the insertable portion coating composition applied to the insertable portion may have a viscosity of from about 10 centipoise (cP) to about 350 cP. In another example, the insertable portion coating composition applied to the insertable portion has a viscosity from about 20 cP to about 200 cP. In still another example, the insertable portion coating composition applied to the insertable portion has a viscosity from about 30 cP to about 300 cP.

Although an independent electrode domain is described herein, in some examples, sufficient hydrophilicity can be provided in the interference domain and/or enzyme domain (depending on which domain is adjacent to the electroactive surfaces) so as to provide for the full transport of ions in the aqueous environment (e.g. without a distinct electrode domain).

### Non-Mediated System Interference Domain

In some examples, an optional interference domain is provided for non-mediated systems disclosed herein, which generally includes a polymer domain that restricts the flow of one or more interferants to the working electrode. In some examples, the interference domain functions as a molecular sieve that allows analytes and other substances that are to be measured by the electrodes to pass through, while preventing passage of other substances, including interferants such as ascorbate and urea (see U.S. Pat. No. 6,001,067 to Shults). Some known interferants for a glucose-oxidase based electrochemical sensor include acetaminophen, ascorbic acid, bilirubin, cholesterol, creatinine, dopamine, ephedrine, ibuprofen, L-dopa, methyldopa, salicylate, tetracycline, tolazamide, tolbutamide, triglycerides, and uric acid.

Several polymer types that can be utilized as a base material for the interference domain include polyurethanes, polymers having pendant ionic groups (e.g., polyurethane-zwitterion) NAFION™, chitosan, cellulose, or alternating layers of polyallylamine and polyacrylate acid, etc., and polymers having controlled pore size, for example. In one example, the interference domain includes a thin, hydrophobic membrane that is non-swellable and restricts diffusion of low molecular weight species. The interference domain is permeable to relatively low molecular weight substances, such as hydrogen peroxide, but restricts the passage of higher molecular weight substances, including glucose and ascorbic acid. In one example, the interference domain comprises charged species (e.g., polymers with pendent charged groups as disclosed herein) that function to interact with one or more species of the sensing system, such as a cofactor, to reduce or eliminate migration from a domain.

Other systems and methods for reducing or eliminating interference species that can be applied to the membrane system of the present disclosure are described in U.S. Pat. No. 7,816,004 to Muradov et al., U.S. Pat. Appl. Pub. No. 2005/0176136 to Burd et al., U.S. Pat. No. 7,081,195 to Simpson et al., and U.S. Pat. No. 7,715,893 to Kamath et al. In some alternate examples, a distinct interference domain is not included.

In one example, the interference domain is deposited onto the electrode domain (or directly onto the electroactive surfaces when a distinct electrode domain is not included) for a dry film domain thickness of from about 0.05 micron or less to about 20 microns or more. In other examples, the dry film domain thickness is from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns. In further examples, the dry film domain thickness is from about 2, 2.5 or 3 microns to about 3.5, 4, 4.5, or 5 microns. Thicker membranes can also be useful, but, in some examples, thinner membranes have a lower impact on the rate of diffusion of the electroactive species from the enzyme domain to the electrodes.

As discussed herein, when two or more sensors are employed in a sensor system, each sensor optionally includes an interference domain configured to prevent the same interferent(s) from permeating the membrane. In another example, when two or more sensors are employed in a sensor system, each sensor optionally includes an interference domain configured to prevent the different, or overlapping but also different, interferent(s) from permeating the membrane.

### Mediated System—Interference Domains

Some second generation electrochemical analyte sensor technologies (2″-gen) leverage immobilized redox mediators to reduce the overpotential required to detect an analyte. This reduction can be significant in contrast to the typical operating potentials for first generation electrochemical analyte sensors (1st-gen, e.g., those operating on the principle of hydrogen peroxide detection on a catalytic metal surface). As an example, 2nd-gen analyte sensor may be biased between +0.0V and +0.3V verses+0.5 to +0.8V for 1st-gen sensors. However, despite this reduction in operating potential and reduction in susceptibility to electroactive interference from endogenous and pharmacologic agents, these 2nd-generation sensors can still succumb to the undue effect of residual interference.

For example, exemplary 2nd-generation analyte sensors utilize polymer-bound covalently-bound redox mediators (e.g., polyvinyl imidazole (PVI)-OS(4,4′-dimethyl-2,2′-bipyridine)2Cl]+/2+) that reduce the overpotential required for the enzymatic detection of a target analyte. An example of such mediator-based sensors include the systems where undue signal influence arising from the presence of co-circulating endogenous electroactive species can result as evidenced by product labeling warnings regarding large doses of ascorbic acid/ascorbate ion (i.e., Vitamin C), possibly resulting in false hyperglycemia alerts and the like. While charge-selective membranes or further reduction in overpotential may mitigate such interference effects, it may result in a material impact to sensitivity and signal-to-noise figures of merit. Accordingly, at present, mediated electrochemical analyte sensing systems continue to exhibit undue signal influence from endogenous metabolites, such as ascorbic acid.

Thus, the present disclosure includes mediated system interference domains developed for 2nd-gen sensor systems, whether they be continuous glucose monitoring or multianalyte monitoring, e.g., ketone-glucose monitoring, the mediated system interference domains comprising one or more oxidase enzymes, which elicit the enzymatic degradation of an interfering metabolite, or ensemble of metabolites, into a peroxide product, for example hydrogen peroxide. The present disclosure provides domains comprising oxidase enzymes alone or in combination with any of the conventional membranes (electrode, enzyme, resistance domains/layers) used with an indwelling 2nd-generation (e.g., mediated) analyte sensor. Exemplary oxidase enzymes include, for example, ascorbate oxidase or urate oxidase that are configured to catalytically convert an undesired interfering species (e.g., ascorbic acid, uric acid) to a hydrogen peroxide product, which manifests significantly less influence at the bias voltages/overpotentials conventionally applied in 2nd-generation sensing systems. This conversion provides reduced overall concentration of the interfering species at the electrode surface (e.g., trading the flux of the interfering species with the flux of hydrogen peroxide), which provides less detrimental effect to the sensed signal than otherwise would be possible in the presence of the interfering species. The mediated system interference domain can be used alone or in combination with other interference domains, interference membranes, or interference domains, said other interference domains, interference membranes, or interference domains can comprise the same polymer(s) matrix, for example, without the one or more oxidase enzymes, peroxidase or catalase.

In some examples of the mediated system interference domain, the oxidase enzymes can be combined with one or more peroxidase or peroxidase-like enzymes (e.g., horseradish peroxidase, catalase) to further cleave the generated hydrogen peroxide product from the oxidase enzyme(s), thereby rendering peroxide electroactive agent inert and unable to undergo a redox reaction at the electrode surface. The present disclosure includes deployment of the mediated system interference domain in one or more of the electrode domain, the enzyme domain, the resistance domain and the interference membrane. The present disclosure includes deployment of the mediated system interference domain in one or more of the electrode domain, the enzyme domain, the resistance domain and the interference domain.

Thus, in one example, using an exemplary ketone/glucose multianalyte sensor system, can comprise the mediated system interference domain comprising at least one of ascorbate oxidase, urate oxidase, horseradish peroxidase, or catalase is present in an enzyme domain comprising a dehydrogenase enzyme (e.g., beta-hydroxybutyrate dehydrogenase, NADH-acting enzyme (e.g., diaphorase, NAD(P)H dehydrogenase), redox polymer (e.g., PVI-Os(bpy)2Cl), optionally a co-factor (if needed, e.g., NAD+, NADP+) and be optionally crosslinked, e.g., using PEG-DGE, CDI or polycarbodiimide crosslinkers. A resistance domain of a biocompatible material or blend of hydrophobic/hydrophilic polymer, for example PVP/PEG-DGE) can be applied over the enzyme domain/mediated system interference domain.

In another example, using an exemplary ketone/glucose multianalyte sensor system, the mediated system interference domain comprising at least one of ascorbate oxidase, urate oxidase, horseradish peroxidase, or catalase is present in a resistance domain comprising of a biocompatible material or blend of hydrophobic/hydrophilic polymer, for example PVP/PEG-DGE). A separate enzyme domain can be positioned proximal to the electrode and adjacent the mediated system interference domain present in the resistance domain, the enzyme domain comprising dehydrogenase enzyme (e.g., beta-hydroxybutyrate dehydrogenase, NADH-acting enzyme (e.g., diaphorase, NAD(P)H dehydrogenase), redox polymer (e.g., PVI-Os(bpy)2Cl), optionally a co-factor (if needed, e.g., NAD+, NADP+) and be optionally crosslinked, e.g., using PEG-DGE or polycarbodiimide crosslinker.

In another example, using an exemplary ketone/glucose multianalyte sensor system, the mediated system interference domain comprising at least one of ascorbate oxidase, urate oxidase, horseradish peroxidase, or catalase is present between an enzyme domain and at least one electrode surface. A resistance domain of a biocompatible material or blend of hydrophobic/hydrophilic polymer, for example PVP/PEG-DGE, can be applied over the enzyme domain.

In other examples, an exemplary ketone or ketone/glucose multianalyte sensor system that is without a mediator, is provided, as discussed further herein. In one example, an exemplary ketone or ketone/glucose multianalyte sensor system that is without a metal-based mediator, e.g., osmium complexes of biimidazole and/or imidazole ligands, is provided. In one example, an exemplary ketone or ketone/glucose multianalyte sensor system that is without a metal-based mediator, e.g., osmium complexes of biimidazole and/or imidazole ligands, configured to provide amperometric signal at an applied voltage of greater than +0.2 V, greater than or equal to +0.3 V, greater than or equal to +0.4 V, greater than or equal to +0.5 V, or greater than or equal to +0.6 V, is provided. In one example, an exemplary ketone or ketone/glucose multianalyte sensor system that is without a metal-based mediator and includes an interference layer, is provided. In one example, an exemplary ketone or ketone/glucose multianalyte sensor system that is without a metal-based mediator, e.g., osmium complexes of biimidazole and/or imidazole ligands, configured to provide amperometric signal at an applied voltage of greater than +0.2 V, greater than or equal to +0.3 V, greater than or equal to +0.4 V, greater than or equal to +0.5 V, or greater than or equal to +0.6 V, and includes an interference layer, is provided.

### Transducing Element Domain

In one example, the membrane system further includes a transducing element domain, for example an enzyme, RNA, DNA, aptamer, binding protein, etc., disposed more distally from the electroactive surfaces than the interference domain (or electrode domain when a distinct interference is not included). In some examples, the transducing element domain is directly deposited onto the electroactive surfaces (when neither an electrode nor interference domain is included). In one example, the transducing element domain provides an enzyme to catalyze the reaction of the analyte and its co-reactant, as described in more detail below. In some examples, the transducing element domain includes glucose oxidase; however other oxidases, for example, galactose oxidase or uricase, can also be used.

For enzyme-based electrochemical sensors to perform effectively and accurately, the sensor's response is limited by neither enzyme activity nor by co-reactant concentration. Enzymes, including glucose oxidase, can be subject to deactivation as a function of time even in ambient conditions, and this behavior is compensated for in forming the enzyme domain. In some examples, the enzyme domain is constructed of aqueous dispersions of colloidal polyurethane polymers including the enzyme. However, in alternate examples the enzyme domain is constructed from an oxygen-enhancing material, for example, at least one of silicone or fluorocarbon, in order to provide a supply of excess oxygen to ensure that oxygen does not limit the sensing reaction. In some examples, the enzyme is immobilized within the enzyme domain. See U.S. Pat. No. 7,379,765 Petisce et al.

In one example, the transducing element domain is deposited onto the interference domain for a “dry film” domain thickness of from about 0.05 micron or less to about 20 microns or more. In other examples, the dry film domain thickness is from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns. In further examples, the dry film domain thickness is from about 2, 2.5 or 3 microns to about 3.5, 4, 4.5, or 5 microns. “Dry film” thickness refers to the thickness of a film cast from a coating formulation by standard coating techniques and includes post-curing of the film.

However, in some examples, the transducing element domain is deposited onto the electrode domain or directly onto the electroactive surfaces. In some examples, the transducing element domain is deposited by spray or dip-coating, slot die coating, 3D printing, pico jet printing, piezo inkjet printing, and the like. In further examples, the transducing element domain is formed by dip-coating the electrode domain into an transducing element domain solution and curing the transducing element domain for from about 15 to about 30 minutes at a temperature of from about 40 to about 55° C. (and can be accomplished under vacuum (e.g., 20 to 30 mmHg)). In examples wherein dip-coating is used to deposit the transducing element domain at room temperature, a insertion rate of from about 1 inch per minute to about 3 inches per minute, with a dwell time of from about 0.5 minutes to about 2 minutes, and a withdrawal rate of from about 0.25 inch per minute to about 2 inches per minute provide a functional coating. However, values outside of those set forth above can be acceptable or even desirable in certain examples, for example, dependent upon viscosity and surface tension as is appreciated by a person of ordinary skill in the art. In one example, the transducing element domain is formed by dip-coating two times (namely, forming two layers) in a coating solution and curing at 50° C. under vacuum for 20 minutes. However, in some examples, the transducing element domain can be formed by dip-coating and/or spray-coating one or more layers at a predetermined concentration of the coating solution, insertion rate, dwell time, withdrawal rate, and/or desired thickness. In yet another example, the transducing element layer is formed from multiple interlayers deposited by self-assembling-monolayers (SAMs) which are usually formed by immersion in the solution that facilitates the surface chemistry. A substrate may be disposed in this solution for a period of time from about 30 minutes to about 24 hours to form the desired transducing element layer to a predetermined thickness. In another example, the substrate may be disposed in this solution for a period of time from about 1 hour to about 18 hours. In another example, the substrate may be disposed in this solution for a period of time from about 3 hours to about 12 hours.

In still other example, the transducing element layer is formed from multiple interlayers. One or more interlayers of the transducing element layer may be varied alone or in combination in various aspects such as chemistry (composition), thickness, or other mechanical, electrical, biological, or other material properties to achieve a target electron mobility or a range of electron mobility through each interlayer.

### Resistance Domain

In one example, the membrane system includes a resistance domain disposed more distal from the electroactive surfaces than the enzyme domain. Although the following description is directed to a resistance domain for a glucose sensor, the resistance domain can be modified for facilitating detection of the concentrations(s) of other analytes and co-reactants as well. In one example, the resistance domain is configured to control the flux of oxygen through the membrane. In another example, the resistance domain is configured to control the flux of an analyte or co-reactant other than oxygen through the membrane. In yet another example, the resistance domain is configured to control the flux of two or more analytes through the membrane.

An immobilized enzyme-based glucose sensor employing oxygen as co-reactant is supplied with oxygen in non-rate-limiting excess in order for the sensor to respond linearly to changes in glucose concentration, while not responding to changes in oxygen concentration. Specifically, when a glucose-monitoring reaction is oxygen limited, linearity is not achieved above minimal concentrations of glucose. Without a semipermeable membrane situated over the enzyme domain to control the flux of glucose and oxygen, a linear response to glucose levels can be obtained only for glucose concentrations of up to about 40 mg/dL. However, in a clinical setting, a linear response to glucose levels is desirable up to at least about 400 mg/dL.

In one example, the resistance domain includes a semi-permeable membrane that controls the flux of oxygen and glucose to the underlying enzyme domain, rendering oxygen in a non-rate-limiting excess. As a result, the upper limit of linearity of glucose measurement is extended to a much higher value than that which is achieved without the resistance domain. In one example, the resistance domain exhibits an oxygen to glucose permeability ratio of from about 50:1 or less to about 400:1 or more. In further examples, the oxygen to glucose permeability ratio is about 200:1.

In alternate examples, a lower ratio of oxygen-to-glucose can be sufficient to provide excess oxygen by using a high oxygen solubility domain (for example, a silicone or fluorocarbon-based material or domain) to enhance the supply/transport of oxygen to the transducing element domain. If more oxygen is supplied to the enzyme, then more glucose can also be supplied to the transducing element without creating an oxygen rate-limiting excess. In alternate examples, the resistance domain is formed from a silicone composition, such as is described in U.S. Pat. Appl. Pub. No. 2005/0090607 to Tapsak et al.

In one example, the resistance domain includes a polyurethane membrane with both hydrophilic and hydrophobic regions. The hydrophilic and hydrophobic regions may be used in combination to control the diffusion of an analyte or analytes (e.g., glucose, oxygen, ketones, lactate, uric acid, etc.) to an analyte sensor. A suitable hydrophobic polymer component is a polyurethane, or polyurethane urea. Polyurethane is a polymer produced by the condensation reaction of a diisocyanate and a difunctional hydroxyl-containing material. A polyurethane urea is a polymer produced by the condensation reaction of a diisocyanate and a difunctional amine-containing material. In polyurethanes and polyurethane urea polymers, either of the hard segment or soft segment can comprise a plurality of distinct chemical structures, e.g., a soft segment can comprise hydrophobic and hydrophilic segments.

Example diisocyanates useful as the hard segment component of polyurethane or polyurethane urea polymers of the present disclosure include aliphatic diisocyanates containing from about 4 to about 8 methylene units. Diisocyanates containing cycloaliphatic moieties can also be useful in the preparation of the polymer and copolymer components of the membranes of the present disclosure. The material that forms the basis of the hydrophobic matrix of the resistance domain may be selected to exhibit sufficient permeability to allow relevant compounds to pass through it, for example, to allow an oxygen molecule to pass through the membrane from the sample under examination in order to reach the active enzyme or electrochemical electrodes. Examples of materials which can be used to make non-polyurethane type membranes include vinyl polymers (including polyvinylimidazole and poly vinylpyridine), polyethers, polyesters, polyamides, inorganic polymers such as polysiloxanes and polycarbosiloxanes, natural polymers such as cellulosic and protein-based materials, and mixtures or combinations thereof. In some examples, these non-polyurethane type membranes include a crosslinking agent in addition to the base polymer, in order to improve mechanical properties and/or tune mass transport of analyte or other species. In some examples, the resistance domain may polyvinyl butyral (PVB). In other examples, the base polymer can be a segmented block copolymer. In another example, the hard segments may be from about 15 wt. % to about 75 wt. %. In yet another example, the hard segments may be from about 25 wt. % to about 55 wt. %. In yet another example, the hard segments may be from about 35 wt. % to about 45 wt. %. For example, the base polymer can comprise polyurethane and/or polyurea segments and one or more of polycarbonate, polydimethylsiloxane (PDMS), polyether, fluoro-modified segments, perfluoropolyols, or polyester segments. In other examples, the base polymer can be a polyurethane copolymer chosen from the group including a polyether-urethane-urea, polycarbonate urethane, polyether-urethane, polyester-urethane, and/or copolymers thereof.

In one example, the hydrophilic polymer component of the resistance domain is polyethylene oxide. For example, one useful hydrophobic-hydrophilic copolymer component is a polyurethane polymer that includes from about 1 wt. % to about 50 wt. % polyethylene oxide (PEO). In one example, the resistance domain includes 5 wt. % to about 30 wt. % polyethylene oxide (PEO). In another example, the resistance domain includes from about 10 wt. % to about 40 wt. % PEO. The polyethylene oxide portions of the copolymer are thermodynamically driven to separate from the hydrophobic portions of the copolymer and the hydrophobic polymer component. The polyethylene oxide-based soft segment portion of the copolymer used to form the final blend affects the water pick-up and subsequent glucose permeability of the membrane.

In one example, one or more of NBDI, IPDI, TDI, MPDI, HMDI, MDI, 1,3-H6XDI, 1,4-H6XDI, CHDI, PPDI, TODI, or HDI, diisocyanates are used to form various polyurethanes and polyurethane ureas for the resistance domain and/or other sensor domains. In one example, the polyurethanes and polyurethane ureas have soft segments that are aliphatic or amphiphilic. In one example, the soft segment is comprised diol, diamine, diester, or dicarbonate. In one example, the soft segment is comprised of a plurality of two or more of diol, diamine, diester, or dicarbonate.

In one example, one or more of NBDI, IPDI, TDI, MPDI, HMDI, MDI, 1,3-H6XDI, 1,4-H6XDI, CHDI, PPDI, TODI, HDI, is reacted with one or more dicarbonates, polyethers, polyesters, polyalkyl-diols or polyakyl-diamines.

In one example, one or more of NBDI, IPDI, TDI, MPDI, HMDI, MDI, 1,3-H6XDI, 1,4-H6XDI, CHDI, PPDI, TODI, HDI, is reacted with a C5 or C6 dicarbonate, for example U90 OXYMER™, polyhexmethylene carbonate glycol (PHA). In one example, NBDI, IPDI, TDI, MPDI, HMDI, MDI, 1,3-H6XDI, 1,4-H6XDI, CHDI, PPDI, TODI, HDI, or mixtures thereof is reacted with a C5 or C6 dicarbonate, for example U90 OXYMER™ and one or more polyethers, polyesters, polyalkyl-diols or polyakyl-diamines. In one example, the dicarbonate is sterically branched to increase the Tg of the soft segment, for example to provide a Tg around body temperature.

In one example, one or more of hard segment diisocyantes of NBDI, IPDI, TDI, MPDI, HMDI, MDI, 1,3-H6XDI, 1,4-H6XDI, CHDI, PPDI, TODI, HDI, is reacted with a polyether, for example, one or more of polytetramethylene oxide (PTMO), polypropylene oxide (PPO), polyethylene glycol (PEG), polybutadiene diol (PBU) alone or in combination with polydimethylpolysiloxane (PDMS). In one example, the same polyether of different molecular weight (Mw) is used. In one example, two or more polyethers of the same or different Mw are used. In one example, one or more polyethers of the same or different Mw are used in combination with one or more PDMS polymers having the same or different Mw. While not be held to any particular theory, as the molecular weight of the soft segment decreases, phase mixing of different soft segment components increases. In one example, it has been observed that high molecular weight of the soft segment provides for the formation of rich phases, likely due to entropic contributions, among other things.

In one example, one or more of hard segment diisocyanates of NBDI, IPDI, TDI, MPDI, HMDI, MDI, 1,3-H6XDI, 1,4-H6XDI, CHDI, PPDI, TODI, HDI, is reacted with one or more polyesters, for example, polyethylene adipate glycol (PEA), polyteramethylene adipate glycol (PBA), alone or in combination with one or more polyethers, polyalkyl-diols or polyakyl-diamines.

In one example, NBDI, IPDI, TDI, MPDI, HMDI, MDI, 1,3-H6XDI, 1,4-H6XDI, CHDI, PPDI, TODI, HDI, or mixtures thereof is reacted with one or more polyalkyl-diols, alone or in combination with one or more polycarbonates, polyethers, polyesters, or polyakyl-diamines.

In one example, the resistance domain described above is deposited directly onto the electrode surface or onto the enzyme domain in one or more layers to yield a resistance domain thickness of from about 0.05 micron or less to about 20 microns or more. In another example, the total resistance domain thickness is from about 0.05, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns. In another example, the total resistance domain thickness is from about 2, 2.5, or 3 microns to about 3.5, 4, 4.5, or 5 microns. In some examples, the resistance domain is deposited onto the enzyme domain by spray coating or dip-coating, slot die coating, 3D printing, pico jet printing, piezo inkjet printing. In certain examples, spray coating is the deposition technique. The spraying process atomizes and mists the solution, and therefore most or all of the solvent is evaporated prior to the coating material settling on the underlying domain, thereby minimizing contact of the solvent with the enzyme. One additional advantage of spray-coating the resistance domain as described in the present disclosure includes formation of a membrane system that substantially blocks or resists ascorbate (a known electrochemical interferent in hydrogen peroxide-measuring glucose sensors). While not wishing to be bound by theory, it is believed that during the process of depositing the resistance domain as described in the present disclosure, a structural morphology is formed, characterized in that ascorbate does not substantially permeate there through.

### Heterocyclic Resistance Domains and CoFactor Immobilization or Retention Domains

In one example, the cofactor and enzyme are present in a domain, e.g., enzyme and/or resistance domain comprising poly vinylpyridine, poly vinylpyridine-co-styrene, poly vinylpyridine copolymers with vinyl and (meth)acrylic monomers, poly(styrene-co-acrylonitrile), polyvinylimidazoles, or polyvinylimidazole copolymers with vinyl and (meth)acrylic monomers and/or provided as a layer adjacent the electrode domain or the electrode surface. As used herein, “poly vinylpyridine” encompasses poly 2-vinylpyridine, 3-vinylpryidine, 3-vinylpryidine, and alkyl substituted derivatives thereof. Blends and/or graphs of the above polymers can be used. Blends and/or graphs of the above polymers with chitosan, amphiphilic or aliphatic polyurethanes or polyurethane urea, polyols (PEG, PTMO etc.), or zwitterionic polymers can be used. In some examples, polystyrene copolymers with vinyl monomers containing electron withdrawing groups, such as nitrile can be used. In some examples, vinyl polymers with benzene and nitrile functional groups can be used.

In one example, the cofactor and enzyme are present in a domain, e.g., enzyme and/or resistance domain comprising an at least partially cross-linked poly(4-vinylpyridine), polyvinyl pyridine-co-styrene, polyvinyl pyridine copolymers with vinyl and (meth)acrylic monomers, poly(styrene-co-acrylonitrile), polyvinylimidazoles, or polyvinyl imidazole copolymers with vinyl and (meth)acrylic monomers are used as the resistance domain and/or provided as a layer adjacent the electrode domain or the electrode surface. In one example, poly(4-vinylpyridine), polyvinyl pyridine-co-styrene, polyvinyl pyridine copolymers with vinyl and (meth)acrylic monomers, poly(styrene-co-acrylonitrile), polyvinylimidazoles, or polyvinyl imidazole copolymers with vinyl and (meth)acrylic monomers, with or without cross-linking provide for immobilization or retention of one or more cofactors in the resistance domain. In one example, immobilization or retention of the cofactor is via covalent bonding with a function group of the polymer. In another example, immobilization or retention of the cofactor is via a non-covalent interaction, e.g., via equilibrium with a function group of the polymer.

Examples of cofactor immobilization via non-covalent interaction with polymers includes NAD+, for example, with cationic polymers (chitosan, quaternized PVPy, poly zwitterionic polymers, etc.), and/or polymers containing boronic acid functional groups. Thus, in one example, the cofactor and enzyme are present in a domain, e.g., enzyme and/or resistance domain comprising polymers with pendant boronic acid groups that provide for strong dynamic covalent bonding with diol functionalities on cofactors with diol functionality, e.g. NAD, at certain pH, allowing for NAD(H) immobilization or retention. Thus, in one example, the ribose ring structure containing 1,2 diols of the NADH and NAD+ structure are used to associate and/or bind to one or more boronic acid functional groups via at least the covalent interactions as shown in Scheme 1a and Scheme 1b for the NADH form. Similar covalent interactions are envisaged for the NAD+ form.

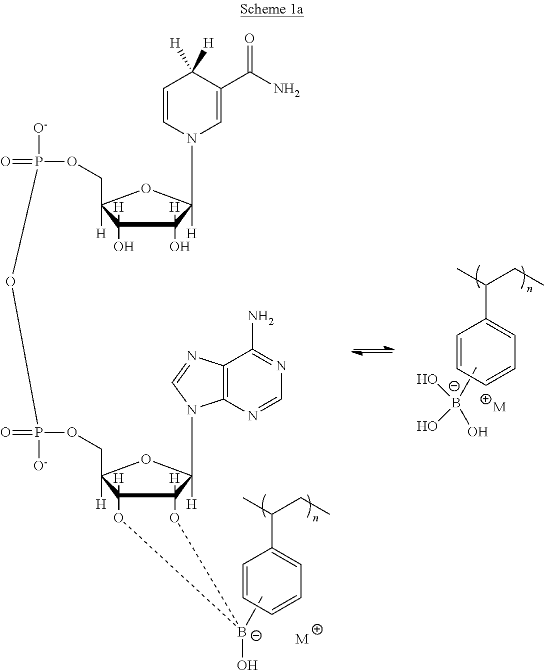

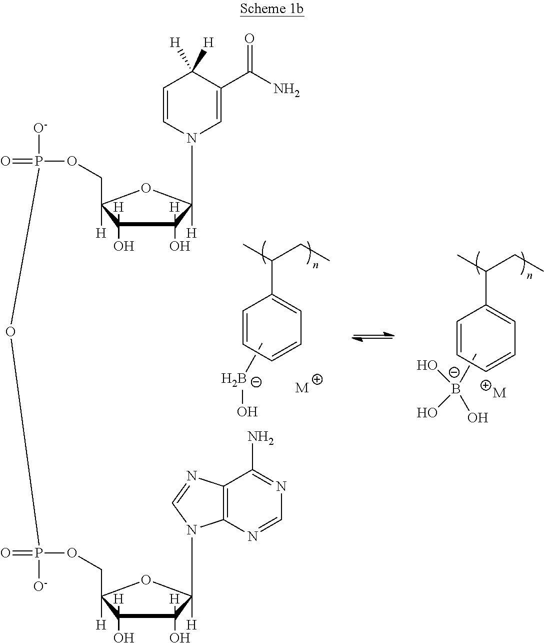

In one example, boronic acid polymer structure and coating solution pH are adjusted to provide sufficient association of the NAD/NADH structure to reduce or eliminate migration from the polymeric membrane. In one example, boronic acid polymers include styrenic polymers, styrenic copolymers (e.g., with acrylic, acrylate, acrylamide, olefinic, cyclic olefinic), naphthyl, anthracenyl polymers and copolymers thereof. In one example, the boronic acid polymer is at least partially crosslinked.

In some examples, the domain is configured to repulse cofactor, for example, the RL functions to “repel” NAD(H) and not let it through, thus attenuating its migration out of the EZL.

In another example, the NAD is tethered to a domain or electrode surface. In another example, the NAD is directly tethered to a domain or directly coupled to an electrode surface. In another example, the NAD is coupled to the electrode surface with an electron transfer agent. In one example, the free amine of the adenine group of the NAD(H) is extended to have an alkyl chain with a primary amine to provide for EDC or (sulfo-)NHS coupling chemistry with a —COOH group on a mediator as shown in Scheme 3, that depicts the modified NAD+ cofactor with an extended free —NH2 coupled to one of the —COOH groups in a PQQ (pyrroloquinoline quinone) mediator.

In another example, the free amine of the adenine group of the NAD(H) is extended to have an alkyl chain with a primary amine to provide for EDC or (sulfo-)NHS coupling chemistry with a HBDH enzyme.

In one example, the modified NAD+ cofactor has an extended free —NH2 which can be readily crosslinked to one of the —COOH groups in a PQQ (pyrroloquinoline quinone) mediator. This PQQ mediator has another —COOH group that can then be crosslinked to a polymer backbone, enzyme, or directly onto the electrode surface.

In one example, the cofactor and enzyme are present in a domain, e.g., enzyme and/or resistance domain comprising an amphiphilic polyurethane or polyurethane urea polymer as disclosed above for the biointerface/drug releasing layer, the aliphatic polyurethane or polyurethane urea having a hard segment content of about 20-40 wt %, a polysiloxane segment of about 10-30 wt %, and a polyglycol segment of about 15-40 wt %. In one example, the amphiphilic polyurethane or polyurethane urea also includes polyvinyl pyrrolidone polymer of 0-25 wt %. In one example, the amphiphilic polyurethane or polyurethane urea also includes polyvinyl pyridine or an alkylated or polyol substituted pyridine polymer of 0-25 wt %. Such amphiphilic polyurethane or polyurethane urea polymer resistance domains provided acceptable sensitivity and greater than two week stability. In one example, the amphiphilic polyurethane or polyurethane urea polymer is at least partially crosslinked. Such amphiphilic polyurethane or polyurethane urea polymer resistance domains provided sensitivity and stability compatible with the PVPy resistance domains discussed above.

In one example, the cofactor and enzyme are present in a domain, e.g., enzyme and/or resistance domain comprising an aliphatic polyurethane or polyurethane urea polymer comprising a hard segment content of about 40-60 wt %, polytetrahydrofuran (PTMO) segments of about 15-50 wt. %, and a polysiloxane segment of about 5-30 wt %. In one example, the aliphatic polyurethane or polyurethane urea polymer is at least partially crosslinked. In one example, polycarbodiimide crosslinkers are used. Such aliphatic polyurethane or polyurethane urea resistance domains showed sensitivity and stability less than the amphiphilic polyurethane or polyurethane urea based resistance domains.

In one example, the cofactor and enzyme are present in a domain, e.g., enzyme and/or resistance domain comprising a block copolymer obtained by polycondensation of a carboxylic acid polyamide (e.g., PA6, PA11, PA12) with a polyether (e.g., polytetramethylene glycol, polyethylene glycol, PEG, polytetrahydrofuran PTMO). In one example, the block copolymer obtained by polycondensation of a carboxylic acid polyamide with a polyether can be at least partially crosslinked.

In one example, the cofactor and enzyme are present in a domain, e.g., enzyme and/or resistance domain, comprising a polyvinyl pyridine-poly(ethylene glycol) diglycidyl ether (PVPy-PEG-DGE) matrix. In one example, the mass ratio of PEG-DGE to PVPy is about 1-10 wt. %. In one example, polyvinyl pyridine-PEG-DGE matrix is at least partially crosslinked.

In one example, the cofactor and enzyme are present in a domain, e.g., enzyme and/or resistance domain, comprising a water dispersible polyurethane-zwitterion polymer crosslinked with a carbodiimide or polycarbodiimide. Examples of such domains include those disclosed in U.S. Pat. Appl. Pub. No. 2017/0191955, U.S. Pat. Appl. Pub. No. 2017/0188922, and U.S. Pat. No. 11,112,377B2, the disclosures of which are incorporated herein by reference. In one example, the domain comprises an enzyme and a polymer comprising polyurethane and/or polyurea segments and one or more zwitterionic repeating units. In one example, the domain comprises an enzyme and a blend of a polyurethane base polymer and polyvinylpyrrolidone. In some examples, the enzyme domains are formed of a polyurethane urea having carboxyl betaine groups incorporated in the polymer and non-ionic hydrophilic polyethylene oxide segments, wherein the polyurethane urea polymer is dissolved in an organic or non-organic solvent system according to a pre-determined coating formulation, and is optionally crosslinked and/or cured. The above described domain can be from 0.01 μm to about 250 μm thick.

Depending upon the example, the resistance domain(s) discussed herein can be formed by any number of methods, for example, but not limited to, dip-coating or spray-coating any layer or plurality of layers, depending upon the concentration of the solution, insertion rate, dwell time, withdrawal rate, and/or the desired thickness of the resulting film, or other factors or combinations of factors.

Advantageously, sensors with the membrane system of the present disclosure, including an electrode domain and/or interference domain, an enzyme domain, and a resistance domain, provide stable signal response to increasing glucose levels of from about 40 to about 400 mg/dL, and sustained function (at least 90% signal strength) even at low oxygen levels (for example, at about 0.6 mg/L O₂). While not wishing to be bound by theory, it is believed that the resistance domain provides sufficient resistivity, or the enzyme domain provides sufficient enzyme, such that oxygen limitations are seen at a much lower concentration of oxygen as compared to prior art sensors.

In one example, a sensor signal has a current in the picoAmp range, which is described in more detail elsewhere herein. However, the ability to produce a signal with a current in the picoAmp range can be dependent upon a combination of factors, including the electronic circuitry design (e.g., A/D converter, bit resolution, and the like), the membrane system (e.g., permeability of the analyte through the resistance domain, enzyme concentration, and/or electrolyte availability to the electrochemical reaction at the electrodes), and the exposed surface area of the working electrode. For example, the resistance domain can be designed to be more or less restrictive to the analyte depending upon to the design of the electronic circuitry, membrane system, and/or exposed electroactive surface area of the working electrode.

Accordingly, in one example, the membrane system is designed with a sensitivity of from about 1 pA/mg/dL to about 100 pA/mg/dL. In other examples, the sensitivity is from about 5 pA/mg/dL to 25 pA/mg/dL. In further examples, the sensitivity is from about 4 to about 7 pA/mg/dL. While not wishing to be bound by any particular theory, it is believed that membrane systems designed with a sensitivity in the above ranges permit measurement of the analyte signal in low analyte and/or low oxygen situations. Namely, conventional analyte sensors have shown reduced measurement accuracy in low analyte ranges due to lower availability of the analyte to the sensor and/or have shown increased signal noise in high analyte ranges due to insufficient oxygen necessary to react with the amount of analyte being measured. While not wishing to be bound by theory, it is believed that the membrane systems of the present disclosure, in combination with the electronic circuitry design and exposed electrochemical reactive surface area design, support measurement of the analyte in the picoAmp range, which enables an improved level of resolution and accuracy in both low and high analyte ranges not seen in the prior art.

Although sensors of some examples described herein include an optional interference domain in order to block or reduce one or more interferants, sensors with the membrane system of the present disclosure, including an electrode domain, an enzyme domain, and a resistance domain, have been shown to inhibit ascorbate without an additional interference domain. Namely, the membrane system of the present disclosure, including an electrode domain, an enzyme domain, and a resistance domain, has been shown to be substantially non-responsive to ascorbate in physiologically acceptable ranges. While not wishing to be bound by theory, it is believed that the process of depositing the resistance domain by spray coating, as described herein, results in a structural morphology that is substantially resistance resistant to ascorbate.

### Interference-Free Membrane Systems

In general, it is believed that appropriate solvents and/or deposition methods can be chosen for one or more of the domains of the membrane system that form one or more transitional domains such that interferants do not substantially permeate therethrough. Thus, sensors can be built without distinct or deposited interference domains, which are non-responsive to interferants. While not wishing to be bound by theory, it is believed that a simplified multilayer membrane system, more robust multilayer manufacturing process, and reduced variability caused by the thickness and associated oxygen and glucose sensitivity of the deposited micron-thin interference domain can be provided. Additionally, the optional polymer-based interference domain, which usually inhibits hydrogen peroxide diffusion, is eliminated, thereby enhancing the amount of hydrogen peroxide that passes through the membrane system. In other examples, the interference domain can be configured to block or reduce the diffusion of one or more interfering species, including H₂O₂, acetaminophen, or other interferents or combinations of interferents.

### Oxygen Conduit

As described above, some sensors employ transducing element within the membrane system through which the host's bodily fluid passes and in which the analytes (for example, glucose, ketone) within the bodily fluid reacts in the presence of a co-reactant (for example, oxygen) to generate a product(s). The product is then measured using electrochemical methods, and thus the output of an electrode system functions as a measure of the analyte. For example, when the sensor is a glucose oxidase based glucose sensor, the species measured at the working electrode is H₂O₂. An enzyme, glucose oxidase, catalyzes the conversion of oxygen and glucose to hydrogen peroxide and gluconate according to the following reaction:

Glucose+O₂→Gluconate+H₂O₂.

Because for each glucose molecule reacted there is a proportional change in the product, H₂O₂, one can monitor the change in H₂O₂ to determine glucose concentration. Oxidation of H₂O₂ by the working electrode is balanced by reduction of ambient oxygen, enzyme generated H₂O₂ and other reducible species at a counter electrode, for example. See Fraser, D. M., “An Introduction to In vivo Biosensing: Progress and Problems.” In “Biosensors and the Body,” D. M. Fraser, ed., 1997, pp. 1-56 John Wiley and Sons, New York.

In vivo, glucose concentration is generally about one hundred times or more than that of the oxygen concentration. Consequently, oxygen is a limiting reactant in the electrochemical reaction, and when insufficient oxygen is provided to the sensor, the sensor is unable to accurately measure glucose concentration. Thus, depressed sensor function or inaccuracy is believed to be a result of problems in availability of oxygen to the enzyme and/or electroactive surface(s).

Accordingly, in an alternate example, an oxygen conduit (for example, a high oxygen solubility domain formed from silicone or fluorochemicals or perfluorocarbon compound) is provided that extends from the ex vivo portion of the sensor to the in vivo portion of the sensor to increase oxygen availability to the enzyme. The oxygen conduit can be formed as a part of the coating (insulating) material or can be a separate conduit associated with the assembly of wire(s) that forms the sensor.

In some examples, one or more domains of the sensing membranes are formed from materials such as silicone, polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, poly(ethylene oxide), poly(propylene oxide) and copolymers and blends thereof, polysulfones and block copolymers thereof including, for example, di-block, tri-block, alternating, random and graft copolymers. U.S. Pat. Appl. Pub. No. 2005/0245799 to Brauker et al., which is incorporated herein by reference in its entirety, describes biointerface and sensing membrane configurations and materials that may be applied to the presently disclosed sensor.

The sensing membrane can be deposited on the electroactive surfaces of the electrode material using known thin or thick film techniques (for example, spraying, electro-depositing, dipping, or the like). It is noted that the sensing membrane that surrounds the working electrode does not have to be the same structure as the sensing membrane that surrounds a reference electrode, etc. For example, the transducing element domain deposited over the working electrode does not necessarily need to be deposited over the reference and/or counter electrodes.

In one example, the sensor is an enzyme-based electrochemical sensor, wherein the working electrode measures the hydrogen peroxide produced by the enzyme catalyzed reaction of glucose being detected and creates a measurable electronic current (for example, detection of glucose utilizing glucose oxidase produces hydrogen peroxide as a by-product, H₂O₂ reacts with the surface of the working electrode producing two protons (2H⁺), two electrons (2e⁻) and one molecule of oxygen (O₂) which produces the electronic current being detected), such as described in more detail above and as is appreciated by a person of ordinary skill in the art. In some examples, one or more potentiostats are employed to monitor the electrochemical reaction at the electroactive surface of the working electrode(s). The potentiostat applies a constant potential to the working electrode and its associated reference electrode to determine the current produced at the working electrode. The current that is produced at the working electrode (and flows through the circuitry to the counter electrode) is substantially proportional to the amount of H₂O₂ that diffuses to the working electrode. The output signal is typically a raw data stream, e.g., a raw signal processed by algorithms prior to display of values, that is used to provide a useful value of the measured analyte concentration in a host to the host or doctor, for example.

Some alternate analyte sensors that can benefit from the systems and methods of the present disclosure include U.S. Pat. No. 5,711,861 to Ward et al., U.S. Pat. No. 6,642,15 to Vachon et al., U.S. Pat. No. 6,654,625 to Say et al., U.S. Pat. No. 6,565,509 to Say et al., U.S. Pat. No. 6,514,718 to Heller, U.S. Pat. No. 6,465,66 to Essenpreis et al., U.S. Pat. No. 6,214,185 to Offenbacher et al., U.S. Pat. No. 5,310,469 to Cunningham et al., and U.S. Pat. No. 5,683,562 to Shaffer et al., U.S. Pat. No. 6,579,690 to Bonnecaze et al., U.S. Pat. No. 6,484,46 to Say et al., U.S. Pat. No. 6,512,939 to Colvin et al., U.S. Pat. No. 6,424,847 to Mastrototaro et al., U.S. Pat. No. 6,424,847 to Mastrototaro et al., for example. Each of the above patents are incorporated in their entirety herein by reference and are not inclusive of all applicable analyte sensors; in general, it should be understood that the disclosed examples are applicable to a variety of analyte sensor configurations. in other examples of sensor systems including biointerface/drug release layer(s), the sensor may be a planar or substantially planar sensor.

### Exemplary Multi-Analyte Sensor Membrane Configurations

Continuous multi-analyte sensors with various membrane configurations suitable for facilitating signal transduction corresponding to analyte concentrations, either simultaneously, intermittently, and/or sequentially are provided. In one example, such sensors can be configured using a signal transducer, comprising one or more transducing elements (“TL”). Such continuous multi-analyte sensor can employ various transducing means, for example, amperometry, voltametric, potentiometry, and impedimetric methods, among other techniques.

In one example, the transducing element comprises one or more membranes that can comprise one or more layers and or domains, each of the one or more layers or domains can independently comprise one or more signal transducers, e.g., enzymes, RNA, DNA, aptamers, binding proteins, etc. As used herein, transducing elements includes enzymes, ionophores, RNA, DNA, aptamers, binding proteins and are used interchangeably.

In one example, the transducing element is present in one or more membranes, layers, or domains formed over a sensing region. In one example, such sensors can be configured using one or more enzyme domains, e.g., membrane domains including enzyme domains, also referred to as EZ layers (“EZLs”), each enzyme domain may comprise one or more enzymes. Reference hereinafter to an “enzyme layer” is intended to include all or part of an enzyme domain, either of which can be all or part of a membrane system as discussed herein, for example, as a single layer, as two or more layers, as pairs of bi-layers, or as combinations thereof.

In one example, the continuous multi-analyte sensor uses one or more of the following analyte-substrate/enzyme pairs: for example, sarcosine oxidase in combination with creatinine amidohydrolase, creatine amidohydrolase being employed for the sensing of creatinine. Other examples of analytes/oxidase enzyme combinations that can be used in the sensing region include, for example, alcohol/alcohol oxidase, cholesterol/cholesterol oxidase, glactose:galactose/galactose oxidase, choline/choline oxidase, glutamate/glutamate oxidase, glycerol/glycerol-3phosphate oxidase (or glycerol oxidase), bilirubin/bilirubin oxidase, ascorbic/ascorbic acid oxidase, uric acid/uric acid oxidase, pyruvate/pyruvate oxidase, hypoxanthine:xanthine/xanthine oxidase, glucose/glucose oxidase, lactate/lactate oxidase, L-amino acid oxidase, and glycine/sarcosine oxidase. Other analyte-substrate/enzyme pairs can be used, including such analyte-substrate/enzyme pairs that comprise genetically altered enzymes, immobilized enzymes, mediator-wired enzymes, dimerized and/or fusion enzymes.

### NAD Based Multi-Analyte Sensor Platform

Nicotinamide adenine dinucleotide (NAD(P)VNAD(P)H) is a coenzyme, e.g., a dinucleotide that consists of two nucleotides joined through their phosphate groups. One nucleotide contains an adenine nucleobase and the other nicotinamide. NAD exists in two forms, e.g., an oxidized form (NAD(P)+) and reduced form (NAD(P)H) (H=hydrogen). The reaction of NAD+ and NADH is reversible, thus, the coenzyme can continuously cycle between the NAD(P)Vand NAD(P)H forms essentially without being consumed.

In one example, one or more enzyme domains of the sensing region of the presently disclosed continuous multi-analyte sensor device comprise an amount of NAD+ or NADH for providing transduction of a detectable signal corresponding to the presence or concentration of one or more analytes. In one example, one or more enzyme domains of the sensing region of the presently disclosed continuous multi-analyte sensor device comprise an excess amount of NAD+ or NADH for providing extended transduction of a detectable signal corresponding to the presence or concentration of one or more analytes.

In one example, NAD, NADH, NAD+, NAD(P)+, ATP, flavin adenine dinucleotide (FAD), magnesium (Mg++), pyrroloquinoline quinone (PQQ), and functionalized derivatives thereof can be used in combination with one or more enzymes in the continuous multi-analyte sensor device. In one example, NAD, NADH, NAD+, NAD(P)+, ATP, flavin adenine dinucleotide (FAD), magnesium (Mg++), pyrroloquinoline quinone (PQQ), and functionalized derivatives are incorporated in the sensing region. In one example, NAD, NADH, NAD+, NAD(P)+, ATP, flavin adenine dinucleotide (FAD), magnesium (Mg++), pyrroloquinoline quinone (PQQ), and functionalized derivatives are dispersed or distributed in one or more membranes or domains of the sensing region.

In one aspect of the present disclosure, continuous sensing of one or more or two or more analytes using NAD+ dependent enzymes is provided in one or more membranes or domains of the sensing region. In one example, the membrane or domain provides retention and stable recycling of NAD+ as well as mechanisms for transducing NADH oxidation or NAD+ reduction into measurable current with amperometry. In one example, described below, continuous, sensing of multi-analytes, either reversibly bound or at least one of which are oxidized or reduced by NAD+ dependent enzymes, for example, ketones (beta-hydroxybutyrate dehydrogenase), glycerol (glycerol dehydrogenase), cortisol (11β-hydroxysteroid dehydrogenase), glucose (glucose dehydrogenase), alcohol (alcohol dehydrogenase), aldehydes (aldehyde dehydrogenase), and lactate (lactate dehydrogenase) is provided. In other examples, described below, membranes are provided that enable the continuous, on-body sensing of multiple analytes which utilize FAD-dependent dehydrogenases, such as fatty acids (Acyl-CoA dehydrogenase).

Exemplary configurations of one or more membranes or portions thereof are an arrangement for providing retention and recycling of NAD+ are provided. Thus, an electrode surface of a conductive wire (coaxial) or a planar conductive surface is coated with at least one layer comprising at least one enzyme as depicted in FIG. 1A. With reference to FIG. 1B, one or more optional layers may be positioned between the electrode surface and the one or more enzyme domains. For example, one or more interference domains (also referred to as “interferent blocking layer”) can be used to reduce or eliminate signal contribution from undesirable species present, or one or more electrodes (not shown) can used to assist with wetting, system equilibrium, and/or start up. As shown in FIGS. 1A, 1B, one or more of the membranes provides a NAD+ reservoir domain providing a reservoir for NAD+. In one example, one or more interferent blocking membranes is used, and potentiostat is utilized to measure H₂O₂ production or O2 consumption of an enzyme such as or similar to NADH oxidase, the NAD+ reservoir and enzyme domain positions can be switched, to facilitate better consumption and slower unnecessary outward diffusion of excess NAD+. Exemplary sensor configurations can be found in U.S. Provisional Patent Application No. 63/321,340, “CONTINUOUS ANALYTE MONITORING SENSOR SYSTEMS AND METHODS OF USING THE SAME,” filed Mar. 18, 2022, and incorporated by reference in its entirety herein; and U.S. Provisional Patent Application No. 63/291,726, “MEDIATOR-TETHERED NAD(H) FOR KETONE SENSING,” filed Dec. 20, 2021, and incorporated by reference in its entirety herein.

In one example, one or more mediators that are optimal for NADH oxidation are incorporated in the one or more electrode domains or enzyme domains. In one example, organic mediators, such as phenanthroline dione, or nitrosoanilines are used. In another example, metallo-organic mediators, such as ruthenium-phenanthroline-dione or osmium(bpy)₂Cl, polymers containing covalently coupled organic mediators or organometallic coordinated mediators polymers for example polyvinylimidizole-Os(bpy)₂Cl, or poly vinylpyridine-organometallic coordinated mediators (including ruthenium-phenanthroline dione) are used. Other mediators can be used as discussed further below.

In humans, serum levels of beta-hydroxybutyrate (BHB) are usually in the low micromolar range but can rise up to about 6-8 mM. Serum levels of BHB can reach 1-2 mM after intense exercise or consistent levels above 2 mM are reached with a ketogenic diet that is almost devoid of carbohydrates. Other ketones are present in serum, such as acetoacetate and acetone, however, most of the dynamic range in ketone levels is in the form of BHB. Thus, monitoring of BHB, e.g., continuous monitoring is useful for providing health information to a user or health care provider.

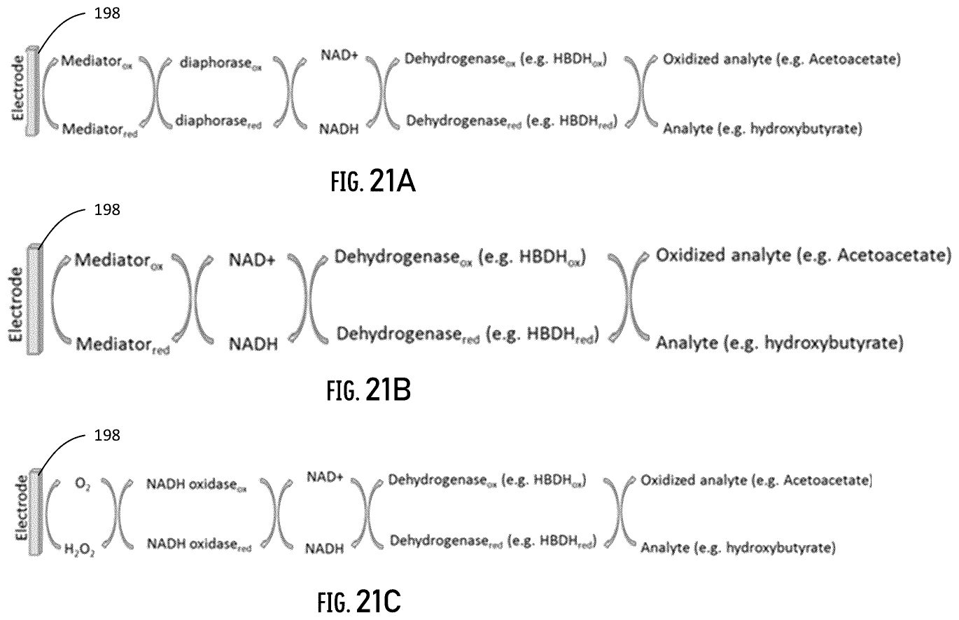

Thus, an exemplary continuous ketone analyte detection employing electrode-associated mediator/NAD+/dehydrogenase, for example, beta-hydroxybutyrate dehydrogenase (HBDH) for continuous monitoring of BHB is provided. In one example, a continuous ketone sensor configuration, capable of monitoring BHB, is depicted in FIG. 21A where a mediator/NAD+/dehydrogenase are present adjacent to the electrode surface 198. Alternatively, for example, multiple enzyme domains can be used in an enzyme layer, with the mediator/NAD+ comprising layer being more proximal to the electrode surface than an adjacent enzyme domain comprising the dehydrogenase enzyme. In one example, the NAD+ and/or HBDH are present in the same or different enzyme domain, and either can be immobilized, for example, using amine reactive crosslinker (e.g., glutaraldehyde, epoxides, NHS esters, imidoesters). In one example, the NAD+ is coupled to a polymer and is present in the same or different enzyme domain as HBDH. In one example, the molecular weight of NAD+ is increased to prevent or reduce migration from the sensing region, for example the NAD+ is dimerized using its C6 terminal amine with any amine-reactive crosslinker, or NAD+ is immobilized to a polymer from its C6 terminal amine. In one example, mediator polymer containing organic mediators, or organometallic coordinated mediator polymers are covalently or otherwise operably coupled to the electrode are used. In other examples, NAD+ may be electrografted to an electroactive surface (e.g., a working electrode). In one example, the electrografted NAD+ is enzyme-active. In another example, the electrografted NAD+ is not enzyme-active.

In some examples, the flux of reactant/co-reactant, such as oxygen through the sensing region has little if any effect on the transduced signal. In the configuration above, there is no consumption of oxygen or production of hydrogen peroxide, rather, direct transfer of electrons from the enzymes to the electrode surface for signal transduction. Thus, notwithstanding endogenous electroactive species such as ascorbate and urate, the need to preferentially attenuate flux of analyte relative to such other reactants such as oxygen and peroxide is reduced or eliminated. For example, homogeneous polymer which have controlled mesh size can be used. In other examples, the sensing region comprises one or more enzyme that is oxygen dependent, and oxygen flux is maximized, for example, including silicone, polysiloxane or copolymers. Other membranes can be used, e.g., positioned in-between or above the aforementioned EZL's or NAD+ reservoirs, for example, drug releasing and/or biointerface layers.

Another example of a continuous ketone analyte detection configuration employing mediator-coupled diaphorase/NAD+/dehydrogenase associated with electrode surface 198 is depicted in FIG. 21B.

In one example, the diaphorase is electrically coupled to the electrode with organometallic coordinated mediator polymer. In another example, the diaphorase is covalently coupled to the electrode with an organometallic coordinated mediator polymer.

Alternatively, multiple enzyme domains can be used in an enzyme layer, for example, separating the electrode-associated diaphorase (closest to the electrode surface) from the more distal adjacent NAD+ or the dehydrogenase enzyme, to essentially decouple NADH oxidation from analyte (ketone) oxidation. Alternatively, NAD+ can be more proximal to the electrode surface than an adjacent enzyme domain comprising the dehydrogenase enzyme. In one example, the NAD+ and/or HBDH are present in the same or different enzyme domain, and either can be immobilized, for example, using amine reactive crosslinker (e.g., glutaraldehyde, epoxides, NHS esters, imidoesters). In one example, the NAD+ is coupled to a polymer and is present in the same or different enzyme domain as HBDH. In one example, the molecular weight of NAD+ is increased to prevent or eliminate migration from the sensing region, for example the NAD+ is dimerized using its C6 terminal amine with any amine-reactive crosslinker. In one example, NAD+ may be covalently coupled to an aspect of the enzyme domain having a higher molecular weight than the NAD+ which may improve a stability profile of the NAD+, improving the ability to retain and/or immobilize the NAD+ in the enzyme domain. For example, dextran-NAD.

In yet another example, transduced signal from a transducing element for a continuous ketone (and one or more other analytes) sensor configuration can be provided using the oxidation of NADH oxidase enzyme for the formation of hydrogen peroxide at electrode surface 198 as the signal transducing species, as illustrated in FIG. 21C. In this configuration, electrode surface 198, membranes, layers, or domains that selectively reduces flux of analyte and NAD+, while allowing high flux of oxygen into the sensing region can be used. Thus, one or more interference domains are used, for example, NAFION™ or alternating layers of polyallylamine and polyacrylate acid, etc. In one example, the HBDH and one or more other analyte-specific oxidase enzymes are present in the same or different enzyme domain, and either can be immobilized.

In one example, the NAD+ may be coupled or non-coupled to a polymer or physically entrapped therein, and is present in the same or different enzyme domain as HBDH. In one example, the molecular weight of NAD+ is increased to prevent or eliminate migration from the sensing region, for example the NAD+ is dimerized using its C6 terminal amine with any amine-reactive crosslinker. In one example, superoxide dismutase (SOD) can be included in the configuration, for example, in the same enzyme domain as NADH for scavenging free radicals generated by NADH oxidase and thus improving signal stability and sensor performance. In the above configuration, the transduced signal is oxygen dependent, and oxygen flux is maximized, for example, using homogeneous polymer membranes which have controlled mesh size, and/or including silicone, polysiloxane or copolymers in the one or more enzyme domains. In one example, where the signal is non-oxygen dependent, a resistance domain is employed to attenuate the flux of analyte(s) into the EZL, so that the sensor response remains linear throughout the physiological range of the target analyte(s). A “target” analyte as discussed herein is an analyte intended to be detected by the sensor systems discussed herein. One or more target analytes may be detected and analyzed using the sensor systems discussed herein.

In one example, the sensing region comprises one or more NADH:acceptor oxidoreductases and one or more NAD-dependent dehydrogenases. In one example, sensing region comprises one or more NADH:acceptor oxidoreductases and one or more NAD(P)-dependent dehydrogenases with NAD(P)+ or NAD(P)H as cofactors present in sensing region. In one example, the sensing region comprises an amount of diaphorase.

In one example, a ketone sensing configuration suitable for combination with another analyte sensing configuration is provided. Thus, an EZL layer of about 1-20 um thick is prepared by presenting a EZL solution composition in 10 mM HEPES in water having about 20 uL 500 mg/mL HBDH, about 20 uL [500 mg/mL NAD(P)H, 200 mg/mL polyethylene glycol-diglycol ether (PEG-DGE) of about 400 MW], about 20 uL 500 mg/mL diaphorase, about 40 uL 250 mg/mL poly vinyl imidazole-osmium bis(2,2′-bipyridine)chloride (PVI-Os(bpy)2Cl) to a substrate such as a working electrode, so as to provide, after drying, about 15-40% by weight HBDH, about 5-30% diaphorase about 5-30% NAD(P)H, about 10-50% PVI-Os(bpy)2Cl and about 1-12% PEG-DGE(400 MW). The substrates discussed herein that may include working electrodes may be formed from gold, platinum, palladium, rhodium, iridium, titanium, tantalum, chromium, and/or alloys or combinations thereof, or carbon (e.g., graphite, glassy carbon, carbon nanotubes, graphene, or doped diamond, as well combinations thereof.

To the above enzyme domain was contacted a resistance domain, also referred to as a resistance layer (“RL”). In one example, the RL comprises about 55-100% polyvinyl pyrrolidone (PVP), and about 0.1-45% PEG-DGE. In another example, the RL comprises about 75-100% PVP, and about 0.3-25% PEG-DGE. In yet another example, the RL comprises about 85-100% PVP, and about 0.5-15% PEG-DGE. In yet another example, the RL comprises essentially 100% PVP.

The exemplary continuous ketone sensor as depicted in FIGS. 1A, 1B comprising NAD(P)H reservoir domain is configured so that NAD(P)H is not rate-limiting in any of the enzyme domains of the sensing region. In one example, the loading of NAD(P)H in the NAD(P)H reservoir domain is greater than about 20%, 30%, 40% or 50% w/w. The one or more of the membranes or portions of one or more membrane domains (hereinafter also referred to as “membranes”) may also contain a polymer or protein binder, such as zwitterionic polyurethane, and/or albumin. Alternatively, in addition to NAD(P)H, the membrane may contain one or more analyte specific enzymes (e.g. HBDH, glycerol dehydrogenase, etc.), so that optionally, the NAD(P)H reservoir membrane also provides a catalytic function. In one example, the NAD(P)H is dispersed or distributed in or with a polymer (or protein), and may be crosslinked to an extent that still allows adequate enzyme/cofactor functionality and/or reduced NAD(P)H flux within the domain.

In one example, NADH oxidase enzyme alone or in combination with superoxide dismutase (SOD) is used in the one or more membranes of the sensing region. In one example, an amount of superoxide dismutase (SOD) is used that is capable of scavenging some or most of one or more free radicals generated by NADH oxidase. In one example, NADH oxidase enzyme alone or in combination with superoxide dismutase (SOD) is used in combination with NAD(P)H and/or a functionalized polymer with NAD(P)H immobilized onto the polymer from a C6 terminal amine in the one or more membranes of the sensing region.

In one example, the NAD(P)H is immobilized to an extent that maintains NAD(P)H catalytic functionality. In one example, dimerized NAD(P)H is used to entrap NAD(P)H within one or more membranes by crosslinking their respective C6 terminal amine together with appropriate amine-reactive crosslinker such as glutaraldehyde or PEG-DGE or polycarbodiimide crosslinker.

The aforementioned continuous ketone sensor configurations can be adapted to other analytes or used in combination with other sensor configurations. For example, analyte(s)-dehydrogenase enzyme combinations can be used in any of the membranes of the sensing region include; glycerol (glycerol dehydrogenase); cortisol (11β-hydroxysteroid dehydrogenase); glucose (glucose dehydrogenase); alcohol (alcohol dehydrogenase); aldehydes (aldehyde dehydrogenase); and lactate (lactate dehydrogenase).

In one example, a semipermeable membrane is used in the sensing region or adjacent thereto or adjacent to one or more membranes of the sensing region so as to attenuate the flux of at least one analyte or chemical species. In one example, the semipermeable membrane attenuates the flux of at least one analyte or chemical species so as to provide a linear response from a transduced signal. In another example, the semipermeable membrane prevents or eliminates the flux of NAD(P)H out of the sensing region or any membrane or domain. In one example, the semipermeable membrane can be an ion selective membrane selective for an ion analyte of interest, such as ammonium ion.

In another example, a continuous multi-analyte sensor configuration comprising one or more enzymes and/or at least one cofactor was prepared. FIG. 1C depicts this exemplary configuration, of an enzyme domain 150 comprising an enzyme (Enzyme) with an amount of cofactor (Cofactor) that is positioned proximal to at least a portion of a working electrode (“WE”) surface, where the WE comprises an electrochemically reactive surface. In one example, a second membrane 151 comprising an amount of cofactor is positioned adjacent the first enzyme domain. The amount of cofactor in the second membrane can provide an excess for the enzyme, e.g., to extend sensor life. One or more resistance domains 152 (“RL”) are positioned adjacent the second membrane (or can be between the membranes). The RL can be configured to block diffusion of cofactor from the second membrane. Electron transfer from the cofactor to the WE transduces a signal that corresponds directly or indirectly to an analyte concentration.

FIG. 1D depicts an alternative enzyme domain configuration comprising a first membrane 151 with an amount of cofactor that is positioned more proximal to at least a portion of a WE surface. Enzyme domain 150 comprising an amount of enzyme is positioned adjacent the first membrane.

In the membrane configurations depicted in FIGS. 1C, 1D, production of an electrochemically active species in the enzyme domain diffuses to the WE surface and transduces a signal that corresponds directly or indirectly to an analyte concentration. In some examples, the electrochemically active species comprises hydrogen peroxide. For sensor configurations that include a cofactor, the cofactor from the first layer can diffuse to the enzyme domain to extend sensor life, for example, by regenerating the cofactor. For other sensor configurations, the cofactor can be optionally included to improve performance attributes, such as stability. For example, a continuous ketone sensor can comprise NAD(P)H and a divalent metal cation, such as Mg⁺². One or more resistance domains RL can be positioned adjacent the second membrane (or can be between the layers). The RL can be configured to block diffusion of cofactor from the second membrane and/or interferents from reaching the WE surface. Other configurations can be used in the aforementioned configuration, such as electrode, resistance, bio-interfacing, and drug releasing membranes, layers or domains. In other examples, continuous analyte sensors including one or more cofactors that contribute to sensor performance.

FIG. 1E depicts another continuous multi-analyte membrane configuration, where {beta}-hydroxybutyrate dehydrogenase BHBDH in a first enzyme domain 153 is positioned proximate to a working electrode WE and second enzyme domain 154, for example, comprising alcohol dehydrogenase (ADH) and NADH is positioned adjacent the first enzyme domain. One or more resistance domains RL 152 may be deployed adjacent to the second enzyme domain 154. In this configuration, the presence of the combination of alcohol and ketone in serum works collectively to provide a transduced signal corresponding to at least one of the analyte concentrations, for example, ketone. Thus, as the NADH present in the more distal second enzyme domain consumes alcohol present in the serum environment, NADH is oxidized to NAD(P)H that diffuses into the first membrane layer to provide electron transfer of the BHBDH catalysis of acetoacetate ketone and transduction of a detectable signal corresponding to the concentration of the ketone. In one example, an enzyme can be configured for reverse catalysis and can create a substrate used for catalysis of another enzyme present, either in the same or different layer or domain. Other configurations can be used in the aforementioned configuration, such as electrode, resistance, bio-interfacing, and drug releasing membranes, layers, or domains. Thus, a first enzyme domain that is more distal from the WE than a second enzyme domain may be configured to generate a cofactor or other element to act as a reactant (and/or a reactant substrate) for the second enzyme domain to detect the one or more target analytes.

Combinations of the above configurations can be employed. FIGS. 1F, 1G depicts experimental data and linear regression, respectively, of the exemplary continuous ketone sensor configuration over a ketone range of 0 mM-8 mM and provides continuous, linear response and signal stability ex-vivo.

FIGS. 1H, 1I and 1J depict experimental data (0-5 mM ketone) demonstrating sensitivity, calibration, and drift (20 hours at 5 mM), respectively, of the exemplary continuous ketone sensor configurations using NAD+ cofactors and NADH oxidases 170, 172 from different natural sources and demonstrates continuous, linear response and signal stability. Data 199 shown in FIGS. 1H-1J represent samples without cofactor (only BHBDH). In one example, mutant NAD+ cofactors are used to improve retention of the cofactor in one or more membranes, provide improved covalent or non-covalent binding of the cofactor with the one or more membranes of the continuous ketone sensor.

### Alcohol Sensor Configurations

In one example, a continuous alcohol (e.g., ethanol) sensor device configuration is provided. In one example, one or more enzyme domains comprising alcohol oxidase (AOX) is provided and the presence and/or amount of alcohol is transduced by creation of hydrogen peroxide, alone or in combination with oxygen consumption or with another substrate-oxidase enzyme system, e.g., glucose-glucose oxidase, in which hydrogen peroxide and or oxygen and/or glucose can be detected and/or measured qualitatively or quantitatively, using amperometry.

In one example, the sensing region for the aforementioned enzyme substrate-oxidase enzyme configurations has one or more enzyme domains comprises one or more electrodes. In one example, the sensing region for the aforementioned enzyme substrate-oxidase enzyme configurations has one or more enzyme domains, with or without the one or more electrodes, further comprises one or interference blocking membranes (e.g. permselective membranes, charge exclusion membranes) to attenuate one or more interferents from diffusing through the membrane to the working electrode. In one example, the sensing region for the aforementioned substrate-oxidase enzyme configurations has one or more enzyme domains, with or without the one or more electrodes, and further comprises one or resistance domains with or without the one or more interference blocking membranes to attenuate one or more analytes or enzyme substrates. In one example, the sensing region for the aforementioned substrate-oxidase enzyme configurations has one or more enzyme domains, with or without the one or more electrodes, one or more resistance domains with or without the one or more interference blocking membranes further comprises one or biointerface membranes and/or drug releasing membranes, independently, to attenuate one or more analytes or enzyme substrates and attenuate the immune response of the host after insertion.

In one example, the one or more interference blocking membranes are deposited adjacent the working electrode and/or the electrode surface. In one example, the one or interference blocking membranes are directly deposited adjacent the working electrode and/or the electrode surface. In one example, the one or interference blocking membranes are deposited between another layer or membrane or domain that is adjacent the working electrode or the electrode surface to attenuate one or all analytes diffusing thru the sensing region but for oxygen. Such membranes can be used to attenuate alcohol itself as well as attenuate other electrochemically actives species or other analytes that can otherwise interfere by producing a signal if they diffuse to the working electrode.

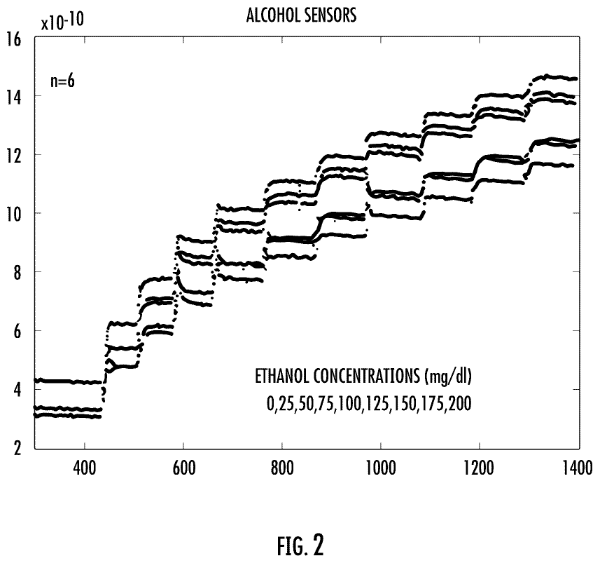

FIG. 2 depicts experimental data from a non-limiting example of an alcohol sensor comprising alcohol oxidase in one or more layers that is coated with one or more interference membranes. In one example, the working electrode used comprised platinum and the potential applied was about 0.5 volts. As shown in FIG. 2, signal response (Amperes) over time (seconds) with stepwise addition of alcohol at ethanol concentrations of from 0 mg/dL to 200 mg/dL. FIG. 2 shows substantial linearity of the current response from 1 to about 200 mg/dL of at least a portion of the pharmacological range of alcohol for a human.

In one example, sensing oxygen level changes electrochemically, for example in a Clark type electrode setup, or in a different configuration can be carried out, for example by coating the electrode with one or more membranes of one or more polymers, such as NAFION™ Based on changes of potential, oxygen concentration changes can be recorded, which correlate directly or indirectly with the concentrations of alcohol. When appropriately designed to obey stoichiometric behavior, the presence of a specific concentration of alcohol should cause a commensurate reduction in local oxygen in a direct (linear) relation with the concentration of alcohol. Accordingly, a multi-analyte sensor for both alcohol and oxygen can therefore be provided.

In another example, the above mentioned alcohol sensing configuration can include one or more secondary enzymes that react with a reaction product of the alcohol/alcohol oxidase catalysis, e.g., hydrogen peroxide, and provide for a oxidized form of the secondary enzyme that transduces an alcohol-dependent signal to the WE/RE at a lower potential than without the secondary enzyme. Thus, in one example, the alcohol/alcohol oxidase is used with a reduced form of a peroxidase, for example horse radish peroxidase. The alcohol/alcohol oxidase can be in same or different layer as the peroxidase, or they may be spatially separated distally from the electrode surface, for example, the alcohol/alcohol oxidase being more distal from the electrode surface and the peroxidase being more proximal to the electrode surface, or alternatively, the alcohol/alcohol oxidase being more proximal from the electrode surface and the peroxidase being more distal to the electrode surface. In one example, the alcohol/alcohol oxidase, being more distal from the electrode surface and the peroxidase, further includes any combination of electrode, interference, resistance, and biointerface membranes to optimize signal, durability, reduce drift, or extend end of use duration.

In another example, the above mentioned alcohol sensing configuration can include one or more mediators. In one example, the one or more mediators are present in, on, or about one or more electrodes or electrode surfaces and/or are deposited or otherwise associated with the surface of the working electrode (WE) or reference electrode (RE). In one example, the one or more mediators eliminate or reduce direct oxidation of interfering species that may reach the WE or RE. In one example, the one or more mediators provide a lowering of the operating potential of the WE/RE, for example, from about 0.6V to about 0.3V or less on a platinum electrode, which can reduce or eliminates oxidation of endogenous interfering species. Examples of one or mediators are provided below. Other electrodes, e.g., counter electrodes, can be employed.

In one example, other enzymes or additional components may be added to the polymer mixture(s) that constitute any part of the sensing region to increase the stability of the aforementioned sensor and/or reduce or eliminate the biproducts of the alcohol/alcohol oxidase reaction. Increasing stability includes storage or shelf life and/or operational stability (e.g., retention of enzyme activity during use). For example, byproducts of enzyme reactions may be undesirable for increased shelf life and/or operational stability, and may thus be desirable to reduce or remove. In one example, xanthine oxidase can be used to remove bi-products of one or more enzyme reactions.

In another example, a dehydrogenase enzyme is used with a oxidase for the detection of alcohol alone or in combination with oxygen. Thus, in one example, alcohol dehydrogenase is used to oxidize alcohol to aldehyde in the presence of reduced nicotinamide adenine dinucleotide (NAD(P)H) or reduced nicotinamide adenine dinucleotide phosphate (NAD(P)+). So as to provide a continuous source of NAD(P)H or NAD(P)+, NADH oxidase or NADPH oxidases is used to oxidize the NAD(P)H or NAD(P)+, with the consumption of oxygen. In another example, Diaphorase can be used instead of or in combination with NADH oxidase or NADPH oxidases. Alternatively, an excess amount of NAD(P)H can be incorporated into the one or more enzyme domains and/or the one or more electrodes in an amount so as to accommodate the intended duration of planned life of the sensor.

In the aforementioned dual enzyme configuration, a signal can be sensed either by: (1) an electrically coupled (e.g., “wired”) alcohol dehydrogenase (ADH), for example, using an electro-active hydrogel polymer comprising one or more mediators; or (2) oxygen electrochemical sensing to measure the oxygen consumption of the NADH oxidase. In an alternative example, the co-factor NAD(P)H or NAD(P)+ may be coupled to a polymer, such as dextran, the polymer immobilized in the enzyme domain along with ADH. This provides for retention of the co-factor and availability thereof for the active site of ADH. In the above example, any combination of electrode, interference, resistance, and biointerface membranes can be used to optimize signal, durability, reduce drift, or extend end of use duration. In one example, electrical coupling, for example, directly or indirectly, via a covalent or ionic bond, to at least a portion of a transducing element, such as an aptamer, an enzyme or cofactor and at least a portion of the electrode surface is provided. A chemical moiety capable of assisting with electron transfer from the enzyme or cofactor to the electrode surface can be used and includes one or more mediators as described below.

In one example, any one of the aforementioned continuous alcohol sensor configurations are combined with any one of the aforementioned continuous ketone monitoring configurations to provide a continuous multi-analyte sensor device as further described below. In one example a continuous glucose monitoring configuration combined with any one of the aforementioned continuous alcohol sensor configurations and any one of the aforementioned continuous ketone monitoring configurations to provide a continuous multi-analyte sensor device as further described below.

### Uric Acid Sensor Configurations

In another example, a continuous uric acid sensor device configuration is provided. Thus, in one example, uric acid oxidase (UOX) can be included in one or more enzyme domains and positioned adjacent the working electrode surface. The catalysis of the uric acid using UOX, produces hydrogen peroxide which can be detected using, among other techniques, amperometry, voltametric and impedimetric methods. In one example, to reduce or eliminate the interference from direct oxidation of uric acid on the electrode surface, one or more electrode, interference, and/or resistance domains can be deposited on at least a portion of the working electrode surface. Such membranes can be used to attenuate diffusion of uric acid as well as other analytes to the working electrode that can interfere with signal transduction.

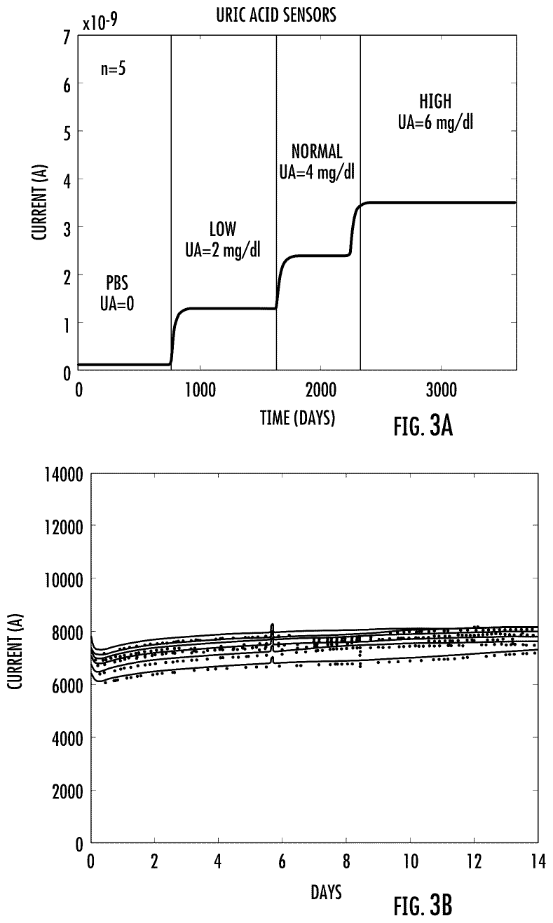

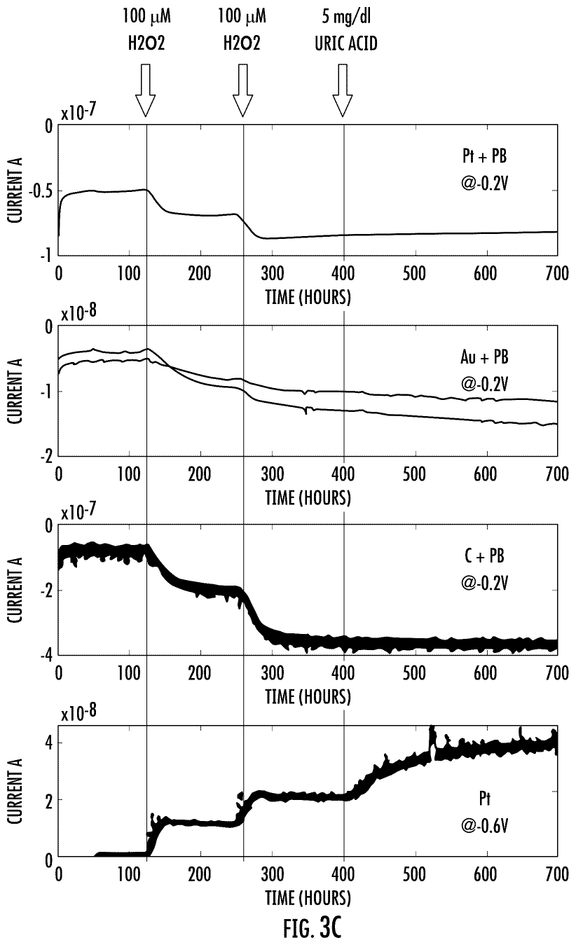

FIGS. 3A, 3B show experimental data from a continuous uric acid sensor configuration. In this example, a platinum working electrode coated with at least one interference membrane, over which there is at least one enzyme domain comprising UOX. The enzyme domain is covered with one or more resistance domains to control or attenuate diffusional characteristics. As shown in FIG. 3A, the exemplary sensors show sensitivity in a physiological concentration range, with stability over drift (at 37° C.; FIG. 3B showing 14 days @ 8 mg/dl UA) as well as high repeatability.

In one alternative example, a uric acid continuous sensing device configuration comprises sensing oxygen level changes about the WE surface, e.g., for example, as in a Clark type electrode setup, or the one or more electrodes can comprise, independently, one or more different polymers such as NAFION™, zwitterion polymers, or polymeric mediator adjacent at least a portion of the electrode surface. In one example, the electrode surface with the one or more electrode domains provide for operation at a different or lower voltage to measure oxygen. Oxygen level and its changes in can be sensed, recorded, and correlated to the concentration of uric acid based using, for example, using conventional calibration methods.

In one example, alone or in combination with any of the aforementioned configurations, uric acid sensor configurations, so as to lower the potential at the WE for signal transduction of uric acid, one or more coatings can be deposited on the WE surface. The one or more coatings may be deposited or otherwise formed on the WE surface and/or on other coatings formed thereon using various techniques including, but not limited to, dipping, electrodepositing, vapor deposition, spray coating, etc. In one example, the coated WE surface can provide for redox reactions, e.g., of hydrogen peroxide, at lower potentials (as compared to 0.6 V on platinum electrode surface without such a coating. Example of materials that can be coated or annealed onto the WE surface includes, but are not limited to Prussian Blue, Medola Blue, methylene blue, methylene green, methyl viologen, ferrocyanide, ferrocene, cobalt ion, and cobalt phthalocyanine, and the like.

With reference to FIG. 3C, graphical depictions of the signal transduction of uric acid in the presence of added hydrogen peroxide with bare or coated electrodes is shown. In the graph, addition of hydrogen peroxide is detected by all electrodes. However, the addition of uric acid is detected at a “bare” platinum electrode (d), in contrast to (a) platinum with electrodeposited Prussian blue, (b) gold with electrodeposited Prussian blue and (c) carbon with electrodeposited Prussian blue, indicating selectiveness of such electrode/enzyme domain configurations. In the above example, any combination of electrode, interference, resistance, and biointerface membranes can be arranged to further optimize signal, durability, reduce drift, or extend end of use duration.

In one example, one or more secondary enzymes, cofactors and/or mediators (electrically coupled or polymeric mediators) can be added to the enzyme domain with UOX to facilitate direct or indirect electron transfer to the WE. In such configurations, for example, regeneration of the initial oxidized form of secondary enzyme is reduced by the WE for signal transduction. In one example, the secondary enzyme is horse radish peroxidase (HRP).

### Choline Sensor Configurations

In one example continuous choline sensor device can be provided, for example, using choline oxidase enzyme that generates hydrogen peroxide with the oxidation of choline. Thus, in one example, at least one enzyme domain comprises choline oxidase (COX) adjacent at least one WE surface, optionally with one or more electrodes and/or interference membranes positioned in between the WE surface and the at least one enzyme domain. The catalysis of the choline using COX results in creation of hydrogen peroxide which can be detectable using, among other techniques, amperometry, voltametric and impedimetric methods.

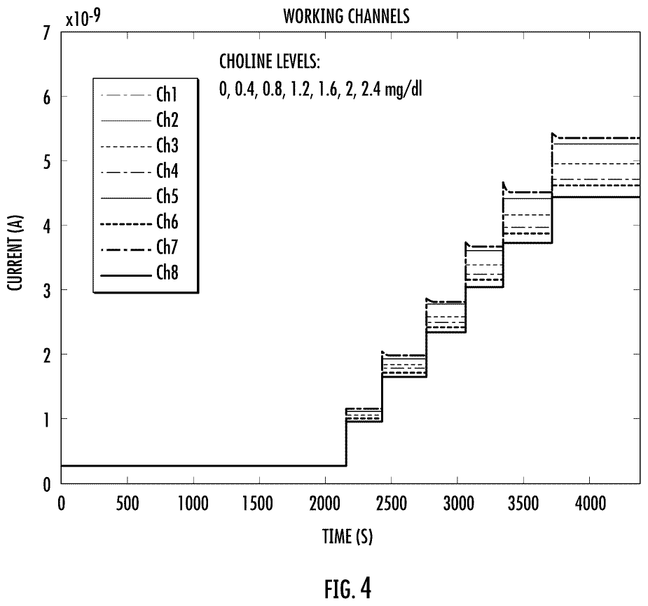

FIG. 4 shows experimental data of an operational choline sensor configuration. In this example, a first enzyme domain is formed over one or more interference membranes adjacent a platinum WE. The first enzyme domain comprising choline oxidase, which is covered with one or more resistance domains to control diffusional characteristics. The data shows the choline sensors can provide sensitivity in physiological concentration ranges, with thermal operational stability over drift (37° C.) as well as high repeatability.

In one example, the aforementioned continuous choline sensor configuration is combined with any one of the aforementioned continuous alcohol sensor configurations, and continuous uric acid sensor configurations to provide a continuous multi-analyte sensor device as further described below. This continuous multi-analyte sensor device can further include continuous glucose monitoring capability. Other membranes can be used in the aforementioned continuous choline sensor configuration, such as electrode, resistance, bio-interfacing, and drug releasing membranes.

### Cholesterol Sensor Configurations

In one example, continuous cholesterol sensor configurations can be made using cholesterol oxidase (CHOX), in a manner similar to previously described sensors. Thus, one or more enzyme domains comprising CHOX can be positioned adjacent at least one WE surface. The catalysis of free cholesterol using CHOX results in creation of hydrogen peroxide which can be detectable using, among other techniques, amperometry, voltametric and impedimetric methods.

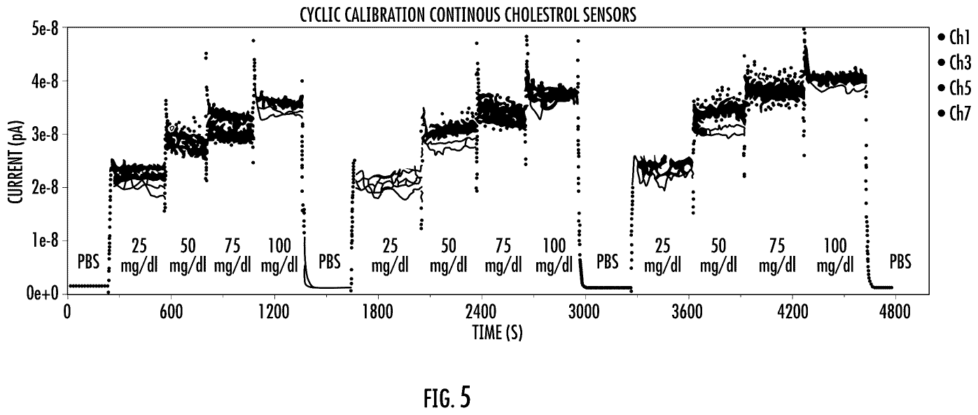

An exemplary cholesterol sensor configuration using a platinum WE, where at least one interference membrane is positioned adjacent at least one WE surface, over which there is at least one enzyme domain comprising CHOX, over which is positioned at least one resistance domain to control diffusional characteristics was prepared. FIG. 5 depicts data demonstrating that the cholesterol sensor configuration above can provide sensitivity in physiological concentration ranges, with thermal operational stability over drift (37° C.) as well as high repeatability.

The method described above and the cholesterol sensors described can measure free cholesterol, however, with modification, the configuration can measure more types of cholesterol as well as total cholesterol concentration. Measuring different types of cholesterol and total cholesterol is important, since due to low solubility of cholesterol in water significant amount of cholesterol is in unmodified and esterified forms. Thus, in one example, a total cholesterol sample is provided where a secondary enzyme is introduced into the at least one enzyme domain, for example, to provide the combination of cholesterol esterase with CHOX Cholesteryl ester, which essentially represents total cholesterols can be measured indirectly from signals transduced from cholesterol present and formed by the esterase.

In one example, the aforementioned continuous (total) cholesterol sensor configuration is combined with any one of the aforementioned continuous alcohol sensor configurations and/or continuous uric acid sensor configurations to provide a continuous multi-analyte sensor system as further described below. This continuous multi-analyte sensor device can further include continuous glucose monitoring capability. Other membrane configurations can be used in the aforementioned continuous cholesterol sensor configuration, such as one or more electrode domains, resistance domains, bio-interfacing domains, and drug releasing membranes.

### Bilirubin Sensor and Ascorbic Acid Sensor Configurations

In one example, continuous bilirubin and ascorbic acid sensors are provided. These sensors can employ bilirubin oxidase and ascorbate oxidase, respectively. However, unlike some oxidoreductase enzymes, the final product of the catalysis of analytes of bilirubin oxidase and ascorbate oxidase is water instead of hydrogen peroxide. Therefore, redox detection of hydrogen peroxide to correlate with bilirubin or ascorbic acid is not possible. However, these oxidase enzymes still consume oxygen for the catalysis, and the levels of oxygen consumption correlates with the levels of the target analyte present. Thus, bilirubin and ascorbic acid levels can be measured indirectly by electrochemically sensing oxygen level changes, as in a Clark type electrode setup, for example.

Alternatively, a different configuration for sensing bilirubin and ascorbic acid can be employed. For example, an electrode domain including one or more electrode domains comprising electron transfer agents, such as NAFION™, zwitterion polymers, or polymeric mediator can be coated on the electrode. Measured oxygen levels transduced from such enzyme domain configurations can be correlated with the concentrations of bilirubin and ascorbic acid levels. In one example, an electrode domain comprising one or more mediators electrically coupled to a working electrode can be employed and correlated to the levels of bilirubin and ascorbic acid levels.

In one example, the aforementioned continuous bilirubin and ascorbic acid sensor configurations can be combined with any one of the aforementioned continuous alcohol sensor configurations, continuous uric acid sensor configurations, continuous cholesterol sensor configurations to provide a continuous multi-analyte sensor device as further described below. This continuous multi-analyte sensor device can further include continuous glucose monitoring capability. Other membranes can be used in the aforementioned continuous bilirubin and ascorbic acid sensor configuration, such as electrode, resistance, bio-interfacing, and drug releasing membranes.

### One-Working-Electrode Configurations for Dual Analyte Detection

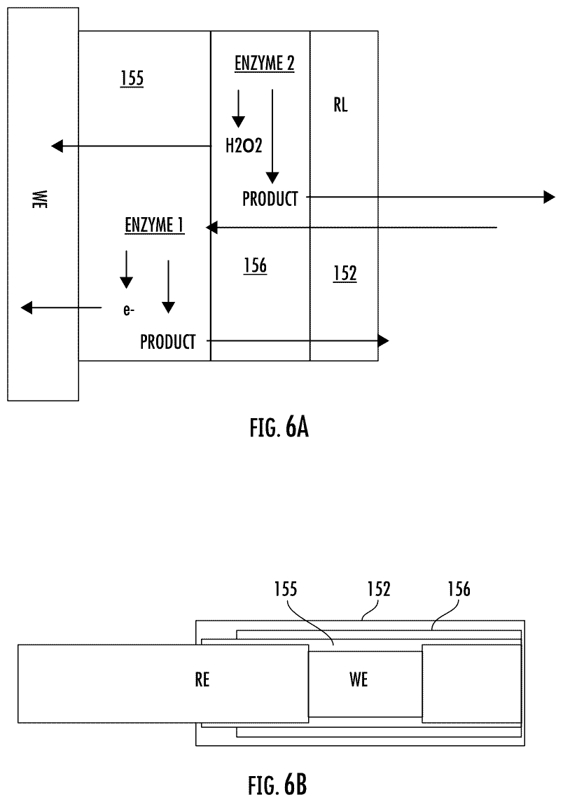

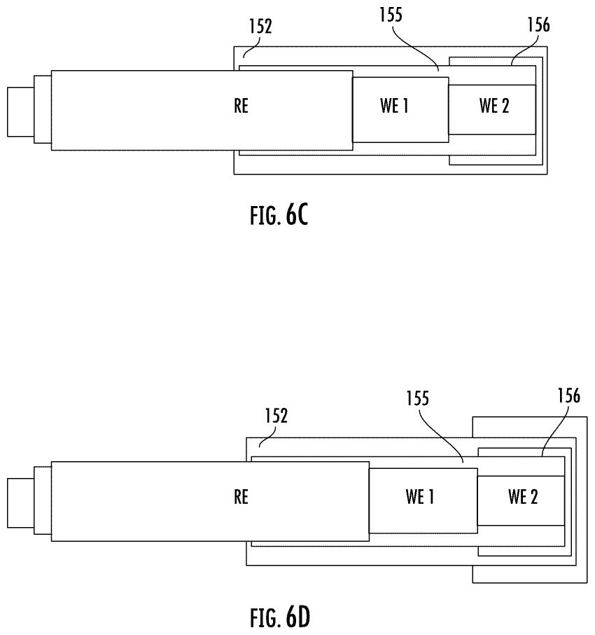

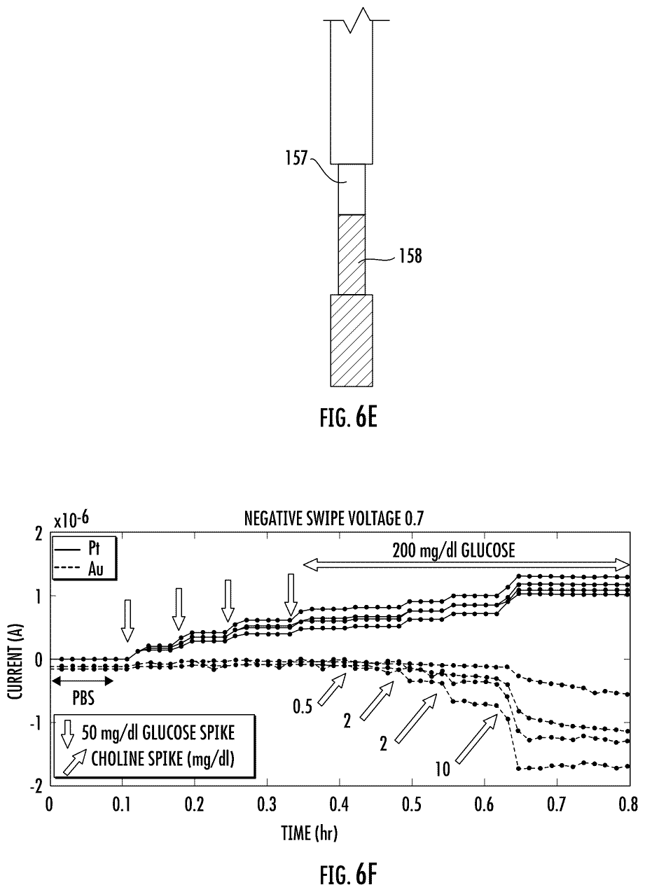

In one example, at least a dual enzyme domain configuration in which each layer contains one or more specific enzymes and optionally one or more cofactors is provided. In a broad sense, one example of a continuous multi-analyte sensor configuration is depicted in FIG. 6A where a first membrane 155 (EZL1) comprising at least one enzyme (Enzyme 1) of the at least two enzyme domain configuration is proximal to at least one surface of a WE. One or more analyte-substrate enzyme pairs with Enzyme 1 transduces at least one detectable signal to the WE surface by direct electron transfer or by mediated electron transfer that corresponds directly or indirectly to an analyte concentration. Second membrane 156 (EZL2) with at least one second enzyme (Enzyme 2) is positioned adjacent 155 ELZ1, and is generally more distal from WE than EZL1. One or more resistance domains (RL) 152 can be provided adjacent EZL2 156, and/or between EZL1 155 and EZL2 156. The different enzymes catalyze the transformation of the same analyte, but at least one enzyme in EZL2 156 provides hydrogen peroxide and the other at least one enzyme in EZL1 155 does not provide hydrogen peroxide. Accordingly, each measurable species (e.g., hydrogen peroxide and the other measurable species that is not hydrogen peroxide) generates a signal associated with its concentration.

For example, in the configuration shown in FIG. 6A, a first analyte diffuses through RL 152 and into EZL2 156 resulting in peroxide via interaction with Enzyme 2. Peroxide diffuses at least through EZL1 155 to WE and transduces a signal that corresponds directly or indirectly to the first analyte concentration. A second analyte, which is different from the first analyte, diffuses through RL 152 and EZL2 156 and interacts with Enzyme 1, which results in electron transfer to WE and transduces a signal that corresponds directly or indirectly to the second analyte concentration.

As shown in FIG. 6B, the above configuration is adapted to a conductive wire electrode construct, where at least two different enzyme-containing layers are constructed on the same WE with a single active surface. In one example, the single WE is a wire, with the active surface positioned about the longitudinal axis of the wire. In another example, the single WE is a conductive trace on a substrate, with the active surface positioned about the longitudinal axis of the trace. In one example, the active surface is substantially continuous about a longitudinal axis or a radius.

In the configuration described above, at least two different enzymes can be used and catalyze the transformation of different analytes, with at least one enzyme in EZL2 156 providing hydrogen peroxide and the at least other enzyme in EZL1 155 not providing hydrogen peroxide, e.g., providing electron transfer to the WE surface corresponding directly or indirectly to a concentration of the analyte.

In one example, an inner layer of the at least two enzyme domains EZL1, EZL2 155, 156 comprises at least one immobilized enzyme in combination with at least one mediator that can facilitate lower bias voltage operation of the WE than without the mediator. In one example, for such direct electron transductions, a potential P1 is used. In one example, at least a portion of the inner layer EZL1 155 is more proximal to the WE surface and may have one or more intervening electrode domains and/or overlaying interference and/or bio-interfacing and/or drug releasing membranes, provided that the at least one mediator can facilitate low bias voltage operation with the WE surface. In another example, at least a portion of the inner layer EZL1 155 is directly adjacent the WE.

The second layer of at least dual enzyme domain (the outer layer EZL2 156) of FIG. 6B contains at least one enzyme that result in one or more catalysis reactions that eventually generate an amount of hydrogen peroxide that can electrochemically transduce a signal corresponding to the concentration of the analyte(s). In one example, the generated hydrogen peroxide diffuses through layer EZL2 156 and through the inner layer EZL1 155 to reach the WE surface and undergoes redox at a potential of P2, where P2≠P1. In this way electron transfer and electrolysis (redox) can be selectively controlled by controlling the potentials P1, P2 applied at the same WE surface. Any applied potential durations can be used for P1, P2, for example, equal/periodic durations, staggered durations, random durations, as well as various potentiometric sequences, cyclic voltammetry etc. In some examples, impedimetric sensing may be used. In one example, a phase shift (e.g., a time lag) may result from detecting two signals from two different working electrodes, each signal being generated by a different EZL (EZL1, EZL2, 155, 156) associated with each electrode. The two (or more) signals can be broken down into components to detect the individual signal and signal artifacts generated by each of EZL1 155 and EZL2 156 in response to the detection of two analytes. In some examples, each EZL detects a different analyte. In other examples, both EZLs detect the same analyte.

In another alternative exemplary configuration, as shown in FIGS. 6C, 6D a multienzyme domain configuration as described above is provided for a continuous multi-analyte sensor device using a single WE with two or more active surfaces is provided. In one example, the multienzyme domain configurations discussed herein are formed on a planar substrate. In another example, the single WE is coaxial, e.g., configured as a wire, having two or more active surfaces positioned about the longitudinal axis of the wire. Additional wires can be used, for example, as a reference and/or counter electrode. In another example, the single WE is a conductive trace on a substrate, with two or more active surfaces positioned about the longitudinal axis of the trace. At least a portion of the two or more active surfaces are discontinuous, providing for at least two physically separated WE surfaces on the same WE wire or trace. (e.g., WE1, WE2), In one example, the first analyte detected by WE1 is glucose, and the second analyte detected by WE2 is lactate. In another example, the first analyte detected by WE1 is glucose, and the second analyte detected by WE2 is ketones.

Thus, FIGS. 6C, 6D depict exemplary configurations of an continuous multi-analyte sensor construct in which EZL1 155, EZL2 156 and RL 152 (resistance domain) as described above, arranged, for example, by sequential dip-coating techniques, over a single coaxial wire comprising spatially separated electrode surfaces WE1, WE2. One or more parameters, independently, of the enzyme domains, resistance domains, etc., can be controlled along the longitudinal axis of the WE, for example, thickness, length along the axis from the distal end of the wire, etc. In one example, at least a portion of the spatially separated electrode surfaces are of the same composition. In another example, at least a portion of the spatially separated electrode surfaces are of different composition. In FIGS. 6C, 6D, WE1 represents a first working electrode surface configured to operate at P1, for example, and is electrically insulated from second working electrode surface WE2 that is configured to operate at P2, and RE represents a reference electrode RE electrically isolated from both WE1, WE2. One resistance domain is provided in the configuration of FIG. 6C that covers the reference electrode and WE1, WE2. An addition resistance domain is provided in the configuration of FIG. 6D that covers extends over essentially WE2 only. Additional electrodes, such as a counter electrode can be used. Such configurations (whether single wire or dual wire configurations) can also be used to measure the same analyte using two different techniques. Using different signal generating sequences as well as different RLs, the data collected from two different mode of measurements provides increase fidelity, improved performance and device longevity. A non-limiting example is a glucose oxidase (H₂O₂ producing) and glucose dehydrogenase (electrically coupled) configuration. Measurement of Glucose at two potentials and from two different electrodes provides more data points and accuracy. Such approaches may not be needed for glucose sensing, but the can be applied across the biomarker sensing spectrum of other analytes, alone or in combination with glucoses sensing, such as ketone sensing, ketone/lactate sensing, and ketone/glucose sensing.

In an alternative configuration of that depicted in FIGS. 6C, 6D, two or more wire electrodes, which can be colinear, wrapped, or otherwise juxtaposed, are presented, where WE1 is separated from WE2, for example, from other elongated shaped electrode. Insulating layer electrically isolates WE1 from WE2. In this configuration, independent electrode potential can be applied to the corresponding electrode surfaces, where the independent electrode potential can be provided simultaneously, sequentially, or randomly to WE1, WE2. In one example, electrode potentials presented to the corresponding electrode surfaces WES1, WES2, are different. One or more additional electrodes can be present such as a reference electrode and/or a counter electrode. In one example, WES2 is positioned longitudinally distal from WES1 in an elongated arrangement. Using, for example, dip-coating methods, WES1 and WES2 are coated with enzyme domain EZL1, while WES2 is coated with different enzyme domain EZL2. Based on the dipping parameters, or different thickness of enzyme domains, multi-layered enzyme domains, each layer independently comprising different loads and/or compositions of enzyme and/or cofactors, mediators can be employed. Likewise, one or more resistance domains (RL) can be applied, each can be of a different thickness along the longitudinal axis of the electrode, and over different electrodes and enzyme domains by controlling dip length and other parameters, for example. With reference to FIG. 6D, such an arrangement of RL's is depicted, where an additional RL 152′ is adjacent WES2 but substantially absent from WES1.

In one example of measuring two different analytes, the above configuration comprising enzyme domain EZL1 155 comprising one or more enzyme(s) and one or more mediators for at least one enzyme of EZL1 to provide for direct electron transfer to the WES1 and determining a concentration of at least a first analyte. In addition, enzyme domain EZL2 156 can comprise at least one enzyme that provides peroxide (e.g., hydrogen peroxide) or consumes oxygen during catalysis with its substrate. The peroxide or the oxygen produced in EZL2 156 migrates to WES2 and provides a detectable signal that corresponds directly or indirectly to a second analyte. For example, WES2 can be carbon, wired to glucose dehydrogenase to measure glucose, while WES1 can be platinum, that measures peroxided produced from lactate oxidase/lactate in EZL2 156. The combinations of electrode material and enzyme(s) as disclosed herein are examples and non-limiting.

In one example, the potentials of P1 and P2 can be separated by an amount of potential so that both signals (from direct electron transfer from EZL1 155 and from hydrogen peroxide redox at WE) can be separately activated and measured. In one example, the electronic module of the sensor can switch between two sensing potentials continuously in a continuous or semi-continuous periodic manner, for example a period (t1) at potential P1, and period (t2) at potential P2 with optionally a rest time with no applied potential. Signal extracted can then be analyzed to measure the concentration of the two different analytes. In another example, the electronic module of the sensor can undergo cyclic voltammetry, providing changes in current when swiping over potentials of P1 and P2 can be correlated to transduced signal coming from either direct electron transfer or electrolysis of hydrogen peroxide, respectably. In one example, the modality of sensing is non limiting and can include different amperometry techniques, e.g., cyclic voltammetry. In one example, an alternative configuration is provided but hydrogen peroxide production in EZL2 is replaced by another suitable electrolysis compound that maintains the P2≠P1 relationship, such as oxygen, and at least one enzyme-substrate combination that provide the other electrolysis compound.

For example, a continuous multi-analyte sensor configuration, for choline and glucose, in which enzyme domains EZ1 155, EZ2 156 were associated with different WEs, e.g., platinum WE2, and gold WE1 was prepared. In this exemplary case, EZL1 155 contained glucose oxidase and a mediator coupled to WE1 to facilitate electron direct transfer upon catalysis of glucose, and EZL2 156 contained choline oxidase that will catalyze choline and generate hydrogen peroxide for electrolysis at WE2. The EZL's were coated with resistance domains; upon cure and readiness they underwent cyclic voltammetry in the presence of glucose and choline. A wired glucose oxidase enzyme to a gold electrode is capable of transducing signal at 0.2 volts, therefore, by analyzing the current changes at 0.2 volts, the concentration of glucose can be determined. The data also demonstrates that choline concentration is also inferentially detectable at the WE2 platinum electrode if the CV trace is analyzed at the voltage P2.

In one example, either electrode WE1 or WE2 can be, for example, a composite material, for example a gold electrode with platinum ink deposited on top, a carbon/platinum mix, and or traces of carbon on top of platinum, or porous carbon coating on a platinum surface. In one example, with the electrode surfaces containing two distinct materials, for example, carbon used for the wired enzyme and electron transfer, while platinum can be used for hydrogen peroxide redox and detection. As shown in FIG. 6E, an example of such composite electrode surfaces is shown, in which an extended platinum covered wire 157 is half coated with carbon 158, to facilitate multi sensing on two different surfaces of the same electrode. In one example WE2 can be grown on or extend from a portion of the surface or distal end of WE1, for example, by vapor deposition, sputtering, or electrolytic deposition and the like.

Additional examples include a composite electrode material that may be used to form one or both of WE1 and WE2. In one example, a platinum-carbon electrode WE1, comprising EZL1 with glucose dehydrogenase is wired to the carbon surface, and outer EZL2 comprising lactate oxidase generating hydrogen peroxide that is detectable by the platinum surface of the same WE1 electrode. Other examples of this configuration can include ketone sensing (beta-hydroxybutyrate dehydrogenase electrically coupled enzyme in EZL1 155) and glucose sensing (glucose oxidase in EZL2 156). Other membranes can be used in the aforementioned configuration, such as electrode, resistance, bio-interfacing, and drug releasing membranes. In other examples, one or both of the working electrodes (WE1, WE2) may be gold-carbon (Au—C), palladium-carbon (Pd—C), iridium-carbon (Ir—C), rhodium-carbon (Rh—C), or ruthenium-carbon (Ru—C). In some examples, the carbon in the working electrodes discussed herein may instead or additionally include graphene, graphene oxide, or other materials suitable for forming the working electrodes, such as commercially available carbon ink.

FIG. 6F graphically depicts obtained sweep traces for the configuration depicted in FIG. 6B, in which the current at 0.7 volt at each swipe of CV is graphed. Glucose was spiked from 50-200 mg/dl, followed by spikes of choline from 0.5-14.5 mg/dl. At 0.7 V, the data indicates that platinum WE2 electrodes (n=4) are sensitive to both glucose and choline, whereas the traces from gold WE1 electrodes (n=4) only show changes in current at 0.7 volt upon choline spikes. This change may be due to formation of hydrogen peroxide in the non-electrically coupled outer EZL2 layer 156 and partial detection of hydrogen peroxide at the gold electrode. Nonetheless, combined with the of detection of glucose at P1 (0.2 Volt) on a gold WE1 electrode, both glucose and choline can be detected using a continuous multi-analyte sensor.

In one example, the first layer EZL1 155 comprises oxidase enzymes that do not produce hydrogen peroxide. Such enzymes include, but are not limited to lactate dehydrogenase, glucose dehydrogenase, beta-hydroxybutyrate dehydrogenase, diaphorase, and the like. In one example, these dehydrogenase enzymes are wired to at least a portion of the WE1 electrode so as to at reduce or eliminate cross talk, reduce potential, and minimize or eliminate interfering signals. In one example the EZL1 155 can comprise any enzyme that can provide electron transfer while wired or covalently coupled to the electrode surface or in the presence of any type of redox mediator, and the EZL2 156 can comprise any oxidoreductase enzymes that produces hydrogen peroxide or other suitable compound that will under redox or electrolysis at the electrode surface at the applied potential.

In one example, the aforementioned continuous choline sensor configurations can be combined with any one of the aforementioned continuous alcohol sensor configurations, continuous uric acid sensor configurations, continuous cholesterol sensor configurations, continuous bilirubin/ascorbic acid sensor configurations, continuous ketone, ketone and glucose, or ketone and lactate, as well as other sensor configurations to provide a continuous multi-analyte sensor device as further described below. This continuous multi-analyte sensor device can further include continuous glucose monitoring capability.

### Glycerol Sensor Configurations

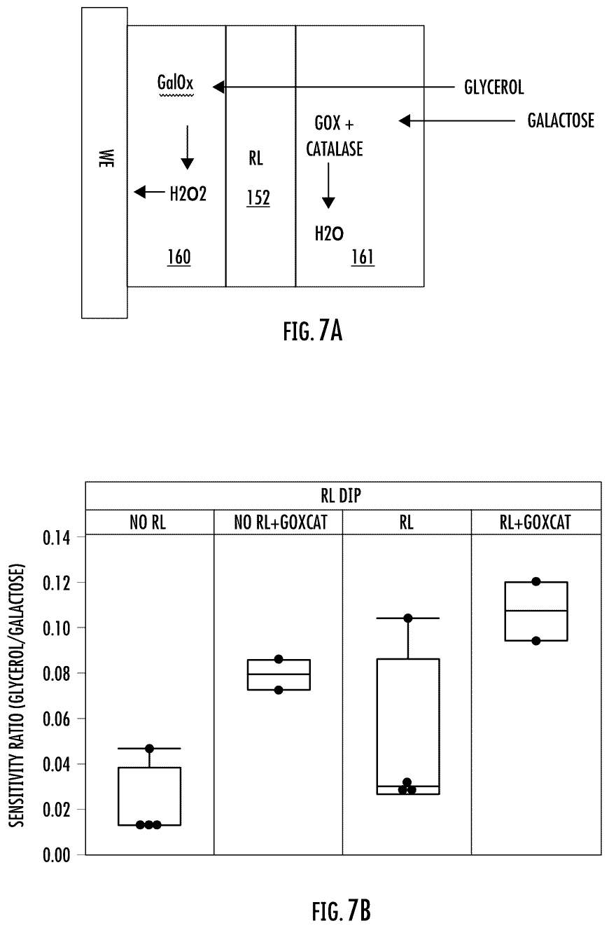

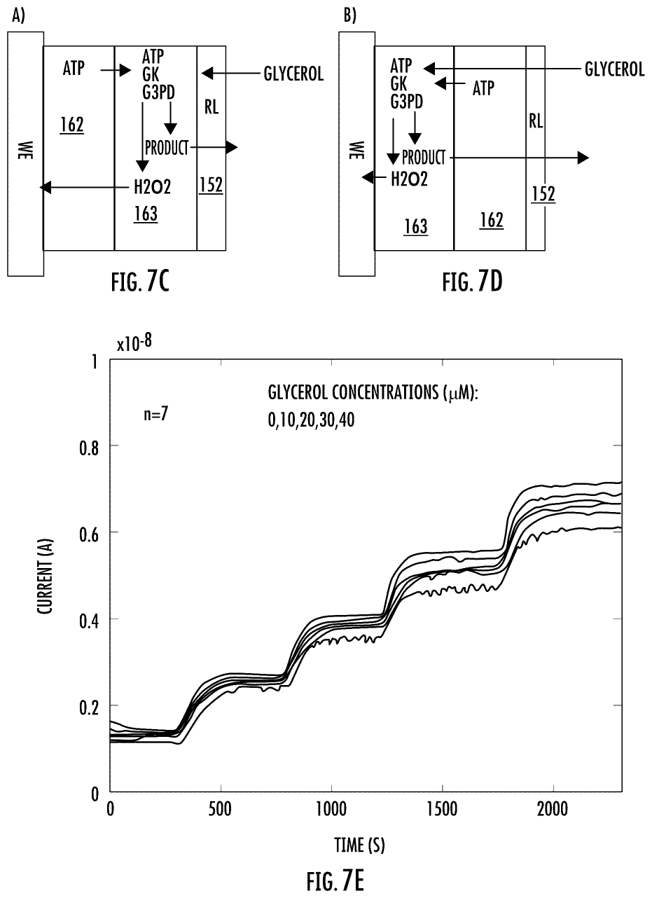

As shown in FIG. 7A, an exemplary continuous glycerol sensor configuration is depicted where a first enzyme domain EZL1 160 comprising galactose oxidase is positioned proximal to at least a portion of a WE surface. A second enzyme domain EZL2 161 comprising glucose oxidase and catalase is positioned more distal from the WE. As shown in FIG. 7A, one or more resistance domains (RL) 152 are positioned between EZL1 160 and EZL2 161. Additional RLs can be employed, for example, adjacent to EZL2 161. Modification of the one or more RL membranes to attenuate the flux of either analyte and increase glycerol to galactose sensitivity ratio is envisaged. The above glycerol sensing configuration provides for a glycerol sensor that can be combined with one or more additional sensor configurations as disclosed herein.

Glycerol can be catalyzed by the enzyme galactose oxidase (GalOx), however, GalOx has an activity ratio of 1%-5% towards glycerol. In one example, the activity of GalOx towards this secondary analyte glycerol can be utilized. The relative concentrations of glycerol in vivo are much higher that galactose (˜2 umol/1 for galactose, and −100 umol/1 for glycerol), which compliments the aforementioned configurations.

If the GalOx present in EZL1 160 membrane is not otherwise functionally limited, then the GalOx will catalyze most if not all of the glycerol that passes through the one or more RLs. The signal contribution from the glycerol present will be higher as compared to the signal contribution from galactose. In one example, the one or more RL's are chemically configured to provide a higher influx of glycerol or a lower influx of galactose.

In another example, a glycol sensor configuration is provided using multiple working electrodes WEs that provides for utilizing signal transduced from both WEs. Utilizing signal transduced from both WEs can provide increasing selectivity. In one example EZL1 160 and EZL2 161 comprise the same oxidase enzyme (e.g., galactose oxidase) with different ratios of enzyme loading, and/or a different immobilizing polymer and/or different number and layers of RL's over the WEs. Such configurations provide for measurement of the same target analyte with different sensitivities, resulting in a dual measurement. Using a mathematical algorithm to correct for noise and interference from a first signal, and inputting the first signal from one sensing electrode with a first analyte sensitivity ratio into the mathematical algorithm, allows for the decoupling of the second signal corresponding to the desired analyte contributions. Modification of the sensitivity ratio of the one or more EZL's to distinguish signals from the interfering species and the analyte(s) of interest can be provided by adjusting one or more of enzyme source, enzyme load in EZL's, chemical nature/diffusional characteristics of EZL's, chemical/diffusional characteristics of the at least one RL's, and combinations thereof.

As discussed herein, a secondary enzyme domain can be utilized to catalyze the non-target analyte(s), reducing their concentration and limiting diffusion towards the sensing electrode through adjacent membranes that contains the primary enzyme and necessary additives. In this example, the most distal enzyme domain, EZL2, 161 is configured to catalyze a non-target analyte that would otherwise react with EZL1, thus providing a potentially less accurate reading of the target analyte (glycerol) concentration. This secondary enzyme domain can act as a “selective diffusion exclusion membrane” by itself, or in some other configurations can be placed above or under a resistant layer (RL) 152. In this example, the target analyte is glycerol and GalOX is used to catalyze glycerol to form a measurable species (e.g., hydrogen peroxide).

FIG. 7B shows sensitivity changes of glycerol sensors in which Galactose Oxidase is used as primary enzyme to convert glycerol and in some cases secondary “selective diffusion exclusion membrane” is used with or without an additional RL layer. As shown in FIG. 7B, application of the selective secondary enzyme domain can increase the selectivity of the sensing platform to the targeted analyte. For example, as shown the sensitivity ratio (sensitivity to targeted analyte/sensitivity to non-targeted analyte) can increase by utilizing RL selective secondary enzyme domain. FIG. 7B shows four dip configurations including: no enzyme or RL (labeled “No RL”), in this configuration as there is neither a secondary enzyme selective layer nor an RL; no RL that includes a second enzyme domain (labeled “No RL+ GOXCAT”), this configuration includes a secondary enzyme domain to catalyze the non-targeted analyte; an RL without an enzyme (labeled “RL”), this configuration only contains resistance domain; and the last configuration includes an RL layer which is placed underneath the selective secondary enzyme domain (and may be described as being positioned in between two EZLs (labeled “RL+GOX-CAT”). “No RL” and “RL” in FIG. 7B indicate the examples including no resistance domain and including a resistance domain, respectively. “No RL+ GOXCAT” and “RL+ GOXCAT” in FIG. 7B indicates example sensors configured to have an outer layer containing GOX and catalase without and with an RL, respectively. An increase in glycerol/galactose sensitivity ratio with smaller variance relative to the RL dip configurations is demonstrated for the RL+ GOXCAT configuration, as compared to the “No RL+ GOXCAT” configuration that did not include an RL.

In one example, a continuous glycerol sensor configuration is provided using at least glycerol oxidase, which provides hydrogen peroxide upon reaction and catalysis of glycerol. Thus, in one example, enzyme domain comprising glycerol oxidase can be positioned adjacent at least a portion of a WE surface and hydrogen peroxide is detected using amperometry. In another example, enzyme domain comprising glycerol oxidase is used for sensing oxygen level changes, for example, in a Clark type electrode setup. Alternatively, at least a portion of the WE surface can be coated with one more layers of electrically coupled polymers, such as a mediator system discussed below, to provide a coated WE capable of electron transfer from the enzyme at a lower potential. The coated WE can then operate at a different and lower voltage to measure oxygen and its correlation to glycerol concentration.

In another example, a glycerol sensor configuration is provided using glycerol-3-phosphate oxidase in the enzyme domain. In one example, ATP is used as the cofactor. Thus, as shown in FIGS. 7C and 7D, exemplary sensor configurations are depicted where in one example (FIG. 7C), one or more cofactors (e.g. ATP) 162 is proximal to at least a portion of an WE surface. One or more enzyme domains 163 comprising glycerol-3-phospohate oxidase (G3PD), lipase, and/or glycerol kinase (GK) and one or more regenerating enzymes capable of continuously regenerating the cofactor are contained in an enzyme domain are adjacent the cofactor, or more distal from the WE surface than the cofactor layer 162. Examples of regenerating enzymes that can be used to provide ATP regeneration include, but are not limited to, ATP synthase, pyruvate kinase, acetate kinase, and creatine kinase. The one or more regenerating enzymes can be included in one or more enzyme domains, or in a separate layer.

An alternative configuration is shown in FIG. 7D, where one or more enzyme domains 163 comprising G3PD, at least one cofactor and at least one regenerating enzyme, are positioned proximal to at least a portion of WE surface, with one or more cofactor reservoirs 162 adjacent to the enzyme domains comprising G3PD and more distal from the WE surface, and one or more RL's 152 are positioned adjacent the cofactor reservoir. In either of these configurations, an additional enzyme domain comprising lipase can be included to indirectly measure triglyceride, as the lipase will produce glycerol for detection by the aforementioned glycerol sensor configurations.

FIG. 7E graphically depicts measured current response to step additions of glycerol levels using a glycerol kinase, glycerol 3 phosphate oxidase and ATP sensor configuration as describe in FIG. 7D, using amperometry at 0.6 volts and in low ranges of physiological concentrations. A “low” range in this example may be based on an expected glycerol value being from about 5 uM to about 100 uM, so a low value may be a value below about 50 uM. In other examples, a low glycerol value may be a value below about 25 uM. In still other examples, a low glycerol value may be below about 10 uM. In yet another example, a low glycerol value may be below about 5 uM.

In another example, a glycerol sensor configuration is provided using dehydrogenase enzymes with cofactors and regenerating enzymes. In one example, cofactors that can be incorporated in the one or more enzyme domains include one or more of NAD(P)H, NADP+, and ATP. In one example, e.g., for use of NAD(P)H a regenerating enzyme can be NADH oxidase or diaphorase to convert NADH, the product of the dehydrogenase catalysis back to NAD(P)H. Similar methodologies can be used for creating other glycerol sensors, for example, glycerol dehydrogenase, combined with NADH oxidase or diaphorase can be configured to measure glycerol or oxygen.

In one example, mathematical modeling can be used to identify and remove interference signals, measuring very low analyte concentrations, signal error and noise reduction so as to improve and increase of multi-analyte sensor end of life. For example, with a two WE electrode configuration where WE1 is coated with a first EZL while WE2 is coated with two or more different EZL, optionally with one or more resistance domains (RL) a mathematical correction such interference can be corrected for, providing for increasing accuracy of the measurements.

Changes of enzyme load, immobilizing polymer and resistance domain characteristics over each analyte sensing region can result in different sensitive ratios between two or more target analyte and interfering species. If the signal are collected and analyzed using mathematical modeling, a more precise concentration of the target analytes can be calculated.

One example in which use of mathematical modeling can be helpful is with glycerol sensing, where galactose oxidase is sensitive towards both galactose and glycerol. The sensitivity ratio of galactose oxidase to glycerol is about is 1%-5% of its sensitivity to galactose. In such case, modification of the sensitivity ratio to the two analytes is possible by adjusting the one or more parameters, such as enzyme source, enzyme load, enzyme domain (EZL) diffusional characteristics, RL diffusional characteristics, and combinations thereof. If two WEs are operating in the sensor system, signal correction and analysis from both WEs using mathematical modeling provides high degree of fidelity and target analyte concentration measurement.

In the above configurations, the proximity to the WE of one or more of these enzyme immobilizing layers discussed herein can be different or reversed, for example if the most proximal to the WE enzyme domain provides hydrogen peroxide, this configuration can be used.

In some examples, the target analyte can be measured using one or multiple of enzyme working in concert. In one example, ATP can be immobilized in one or more EZL membranes, or can be added to an adjacent layer alone or in combination with a secondary cofactor, or can get regenerated/recycled for use in the same EZL or an adjacent third EZL. This configuration can further include a cofactor regenerator enzyme, e.g., alcohol dehydrogenase or NADH oxidase to regenerate NAD(P)H. Other examples of cofactor regenerator enzymes that can be used for ATP regeneration are ATP synthase, pyruvate kinase, acetate kinase, creatine kinase, and the like.

In one example, the aforementioned continuous glycerol sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations to provide a continuous multi-analyte sensor device as further described below. This continuous multi-analyte sensor device can further include continuous glucose monitoring capability. Other configurations can be used in the aforementioned continuous glycerol sensor configuration, such as electrode, resistance, bio-interfacing, and drug releasing membranes.

### Creatinine Sensor Configurations

In one example, continuous creatinine sensor configurations are provided, such configurations containing one or more enzymes and/or cofactors. Creatinine sensor configurations are examples of continuous analyte sensing systems that generate intermediate, interfering products, where these intermediates/interferents are also present in the biological fluids sampled. The present disclosure provides solutions to address these technical problems and provide for accurate, stable, and continuous creatinine monitoring alone or in combination with other continuous multi-analyte sensor configurations.

Creatinine sensors, when in use, are subject to changes of a number of physiologically present intermediate/interfering products, for example sarcosine and creatine, that can affect the correlation of the transduced signal with the creatinine concentration. The physiological concentration range of sarcosine, for example, is an order of magnitude lower that creatinine or creatine, so signal contribution from circulating sarcosine is typically minimal. However, changes in local physiological creatine concentration can affect the creatinine sensor signal. In one example, eliminating or reducing such signal contribution is provided.

Thus, in one example, eliminating or reducing creatine signal contribution of a creatinine sensor comprises using at least one enzyme that will consume the non-targeted interfering analyte, in this case, creatine. For example, two enzyme domains are used, positioned adjacent to each other. At least a portion of a first enzyme domain is positioned proximal to at least a portion of a WE surface, the first enzyme domain comprising one or more enzymes selected from creatinine amidohydrolase (CNH), creatine amidohydrolase (CRH), and sarcosine oxidase (SOX). A second enzyme domain, adjacent the first enzyme domain and more distal from the WE surface, comprises one or more enzymes using creatine as their substrate so as to eliminate or reduce creatine diffusion towards the WE. In one example, combinations of enzymes include CRH, SOX, creatine kinase, and catalase, where the enzyme ratios are tuned to provide ample number of units such that circulating creatine will at least partially be consumed by CRH providing sarcosine and urea, whereas the sarcosine produced will at least partially be consumed by SOX, providing an oxidized form of glycine (e.g. glycine aldehyde) which will at least be partially consumed by catalase. In an alternative configuration of the above, the urea produced by the CRH catalysis can at least partially be consumed by urease to provide ammonia, with the aqueous form (NH4+) being detected via an ion-selective electrode (e.g., nonactin ionophore). Such an alternative potentiometric sensing configuration may provide an alternative to amperometric peroxide detection (e.g., improved sensitivity, limits of detection, and lack of depletion of the reference electrode, alternate pathways/mechanisms). This dual-analyte-sensing example may include a creatinine-potassium sensor having potentiometric sensing at two different working electrodes. In this example, interference signals can be identified and corrected. In one alternative example, the aforementioned configuration can include multi-modal sensing architectures using a combination of amperometry and potentiometry to detect concentrations of peroxide and ammonium ion, measured using amperometry and potentiometry, respectively, and correlated to measure the concentration of the creatinine. In one example, the aforementioned configurations can further comprise one or more configurations (e.g., without enzyme) separating the two enzyme domains to provide complementary or assisting diffusional separations and barriers.

In yet another example, a method to isolate the signal and measure essentially only creatinine is to use a second WE that measures the interfering species (e.g., creatine) and then correct for the signal using mathematical modeling. Thus, for example, signal from the WE interacting with creatine is used as a reference signal. Signal from another WE interacting with creatinine is from corrected for signal from the WE interacting with creatine to selectively determine creatinine concentration.

In yet another example, sensing creatinine is provided by measuring oxygen level changes electrochemically, for example in a Clark type electrode setup, or using one or more electrodes coated with layers of different polymers such as NAFION™ and correlating changes of potential based on oxygen changes, which will indirectly correlate with the concentrations of creatinine.

In yet another example, sensing creatinine is provided by using sarcosine oxidase wired to at least one WE using one or more electrically coupled mediators. In this approach, concentration of creatinine will indirectly correlate with the electron transfer generated signal collected from the WE.

For the aforementioned creatinine sensor configurations based on hydrogen peroxide and/or oxygen measurements the one or more enzymes can be in a single enzyme domain, or the one or more enzymes, independently, can be in one or more enzyme domains, or any other combination thereof, in which in each layer at least one enzyme is present. For the aforementioned creatinine sensor configurations based on use of an electrically coupled sarcosine oxidase containing layer, the layer positioned adjacent to the electrode and is electrically coupled to at least a portion of the electrode surface using mediators.

In another example, the aforementioned creatinine sensor configurations can be sensed using potentiometry by using urease enzyme (UR) that creates ammonium from urea, the urea created by CRH from creatine, the creatine being formed from the interaction of creatinine with CNH. Thus, ammonium can be measured by the above configuration and correlated with the creatinine concentration. Alternatively, creatine amidohydrolase (CI) or creatinine deiminase can be used to create ammonia gas, which under physiological conditions of an transcutaneous sensor, would provide ammonium ion for signal transduction.

In yet another example, sensing creatinine is provided by using one or more enzymes and one or more cofactors. Some non-limiting examples of such configurations include creatinine deaminase (CD) providing ammonium from creatinine, glutamate dehydrogenase (GLDH) providing peroxide from the ammonium, where hydrogen peroxide correlates with levels of present creatinine. The above configuration can further include a third enzyme glutamate oxidase (GLOD) to further break down glutamate formed from the GDLH and create additional hydrogen peroxide. Such combinations of enzymes, independently, can be in one or more enzyme domains, or any other combination thereof, in which in each domain or layer, at least one enzyme is present.

In yet another example, sensing creatinine is provided by the combination of creatinine amidohydrolase (CNH), creatine kinase (CK) and pyruvate kinase (PK), where pyruvate, created by PK can be detected by one or more of either lactate dehydrogenase (LDH) or pyruvate oxidase (PDX) enzymes configured independently, where one or more of the aforementioned enzyme are present in one layer, or, in which in each of a plurality of layers comprises at least one enzyme, any other combination thereof.

In such sensor configurations where one or more cofactors and/or regenerating enzymes for the cofactors are used, providing excess amounts of one or more of NADH, NAD(P)H and ATP in any of the one or more configurations can be employed, and one or more diffusion resistance domains can be introduced to limit or prevent flux of the cofactors from their respective membrane(s). Other configurations can be used in the aforementioned configurations, such as electrode, resistance, bio-interfacing, and drug releasing membranes.

In yet another example, creatinine detection is provided by using creatinine deiminase in one or more enzyme domains and providing ammonium to the enzyme domain(s) via catalysis of creatinine. Ammonium ion can then be detected potentiometrically or by using composite electrodes that undergo redox when exposed to ammonium ion, for example NAFION™/polyaniline composite electrodes, in which polyaniline undergoes redox in the presence of ammonium at the electrode under potential. Ammonium concentration can then be correlated to creatinine concentration.

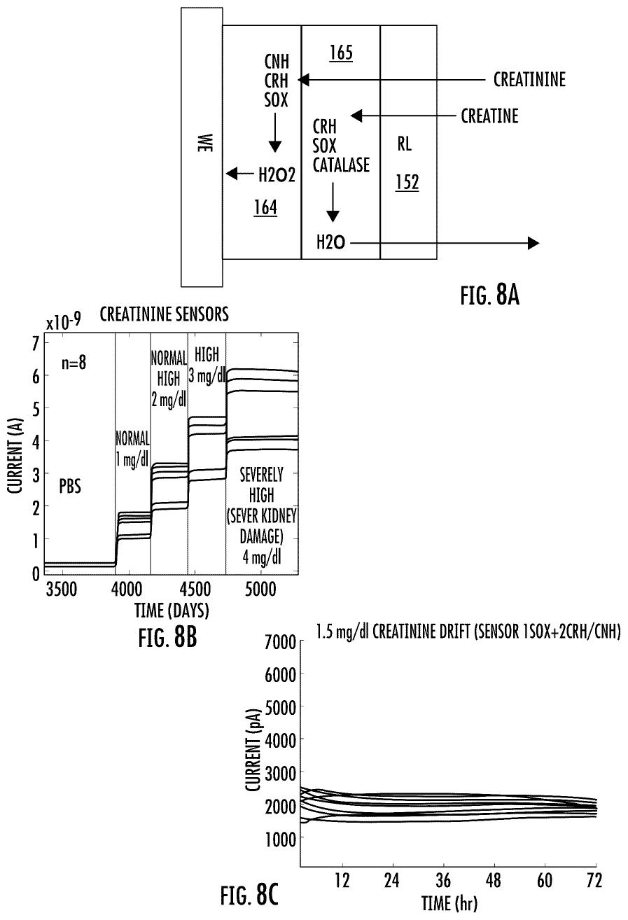

FIG. 8A depicts an exemplary continuous sensor configuration for creatinine. In the example of FIG. 8A, the sensor includes a first enzyme domain 164 comprising CNH, CRH, and SOX are adjacent a working electrode WE, e.g., platinum. A second enzyme domain 165 is positioned adjacent the first enzyme domain and is more distal from the WE. One or more resistance domains (RL) 152 can be positioned adjacent the second enzyme domain or between the first and second layers. Creatinine is diffusible through the RL and the second enzyme domain to the first enzyme domain where it is converted to peroxide and transduces a signal corresponding to its concentration. Creatine is diffusible through the RL and is converted in the second enzyme domain to sarcosine and urea, the sarcosine being consumed by the sarcosine oxidase and the peroxide generated is consumed by the catalase, thus preventing transduction of the creatine signal.

FIG. 8B depicts experimental results of the exemplary creatinine senor with the (CNH, CRH, SOX) configuration discussed above using platinum electrode surfaces. The data demonstrates repeatable sensitivity in creatinine physiological range with up to 7 days of drift stability in the physiological range (FIG. 8C). Such combinations of CNH, CRH, SOX enzymes, independently, can be in one or more enzyme domains, or any other combination thereof, in which in each layer at least one enzyme is present. Moreover, such sensor configurations can include one or more electrode domains, interference membranes, resistance domains so as to modify and adapt sensitivity, selectivity and stability of the sensor.

For example, variations of the above configuration are possible for continuous monitoring of creatinine alone or in combination with one or more other analytes. Thus, one alternative approach to sensing creatinine could be sensing oxygen level changes electrochemically, for example in a Clark-type electrode setup. In one example, the WE can be coated with layers of different polymers, such as NAFION™ and based on changes of potential oxygen changes, the concentrations of creatinine can be correlated. In yet another example, one or more enzyme most proximal to the WE, i.e., sarcosine oxidase, can be “wired” to the electrode using one or more mediators. Each of the different enzymes in the above configurations can be distributed inside a polymer matrix or domain to provide one enzyme domain. In another example, one or more of the different enzymes discussed herein can be formed as the enzyme domain and can be formed layer by layer, in which each layer has at least one enzyme present. In an example of a “wired” enzyme configuration with a multilayered membrane, the wired enzyme domain would be most proximal to the electrode. One or more interferent layers can be deposited among the multilayer enzyme configuration so as to block of non-targeted analytes from reaching electrodes.

In one example, the aforementioned continuous creatinine sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations to provide a continuous multi-analyte sensor device as further described below. This continuous multi-analyte sensor device can further include continuous glucose monitoring capability.

### Lactose Sensor Configurations

In one example, a continuous lactose sensor configuration, alone or in combination with another analyte sensing configuration comprising one or more enzymes and/or cofactors is provided. In a general sense, a lactose sensing configuration using at least one enzyme domain comprising lactase enzyme is used for producing glucose and galactose from the lactose. The produced glucose or galactose is then enzymatically converted to a peroxide for signal transduction at an electrode. Thus, in one example, at least one enzyme domain EZL1 comprising lactase is positioned proximal to at least a portion of a WE surface capable of electrolysis of hydrogen peroxide. In one example, glucose oxidase enzyme (GOX) is included in EZL1, with one or more cofactors or electrically coupled mediators. In another example, galactose oxidase enzyme (GalOx) is included in EZL1, optionally with one or more cofactors or mediators. In one example, glucose oxidase enzyme and galactose oxidase are both included in EZL1. In one example, glucose oxidase enzyme and galactose oxidase are both included in EZL1, optionally with one or more cofactors or electrically coupled mediators.

One or more additional EZL's (e.g. EZL2) can be positioned adjacent the EZL1, where at least a portion of EZL2 is more distal from at least a portion of WE than EZL1. In one example, one or more layers can be positioned in between EZL1 and EZL2, such layers can comprise enzyme, cofactor or mediator or be essentially devoid of one or more of enzymes, cofactors or mediators. In one example, the one or more layers positioned in between EZL1 and EZL2 is essentially devoid of enzyme, e.g., no purposefully added enzyme. In one example one or layers can be positioned adjacent EZL2, being more distal from at least a portion of EZL1 than EZL2, and comprise one or more of the enzymes present in either EZL1 or EZL2.

In one example of the aforementioned lactose sensor configurations, the peroxide generating enzyme can be electrically coupled to the electrode using coupling mediators. The transduced peroxide signals from the aforementioned lactose sensor configurations can be correlated with the level of lactose present.

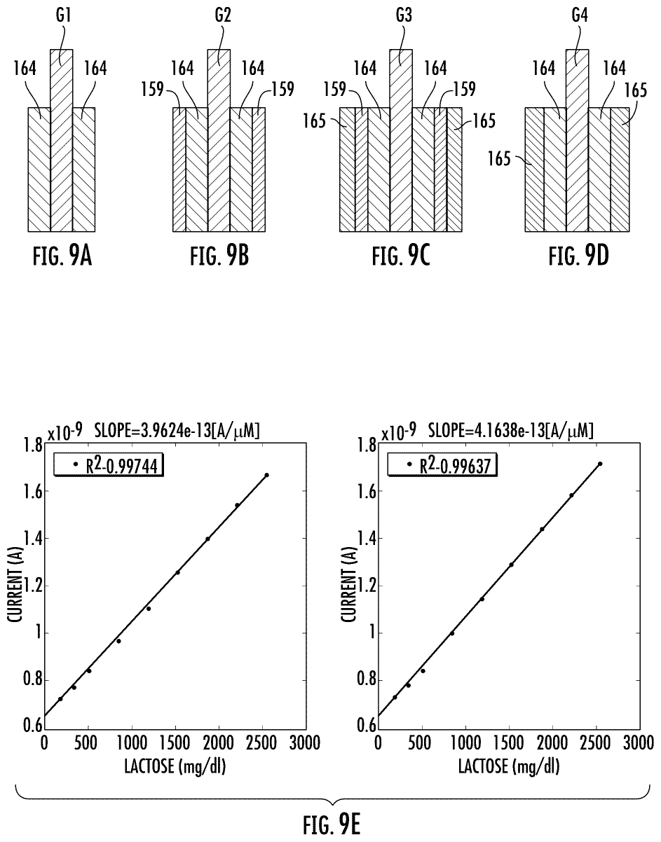

FIGS. 9A, 9B, 9C, and 9D depict alternative continuous lactose sensor configurations. Thus, in an enzyme domain EZL1 164 most proximal to WE (G1), comprising GalOx and lactase, provides a lactose sensor that is sensitive to galactose and lactose concentration changes and is essentially non-transducing of glucose concentration. As shown in FIGS. 9B, 9C, and 9D, additional layers, including non-enzyme containing layers 159, and a lactase enzyme containing layer 165, and optionally, electrode, resistance, bio-interfacing, and drug releasing membranes. (not shown) are used. Since changes in physiological galactose concentration are minimal, the transduced signal would essentially be from physiological lactose fluctuations. FIG. 9E demonstrates a linear response to lactose from the above described configuration depicted in FIG. 9C.

In one example, the aforementioned continuous lactose sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations, creatinine sensor configurations to provide a continuous multi-analyte sensor device as further described below. This continuous multi-analyte sensor device can further include continuous glucose monitoring capability. Other membranes can be used in the aforementioned sensor configuration, such as electrode, resistance, bio-interfacing, and drug releasing membranes.

### Urea Sensor Configurations

Similar approach as described above can also be used to create a continuous urea sensor. For example urease (UR), which can break down the urea and to provide ammonium can be used in an enzyme domain configuration. Ammonium can be detected with potentiometry or by using a composite electrodes, e.g., electrodes that undergo redox when exposed to ammonium. Example electrodes for ammonium signal transduction include, but are not limited to, NAFION™/polyaniline composite electrodes, in which polyaniline undergoes redox in the presence of ammonium at an applied potential, with essentially direct correlation of signal to the level of ammonium present in the surrounding. This method can also be used to measure other analytes such as glutamate using the enzyme glutaminase (GLUS).

In one example, the aforementioned continuous uric acid sensor configurations can be combined with any one of the aforementioned continuous alcohol sensor configurations and/or continuous uric acid sensor configurations and/or continuous cholesterol sensor configurations and/or continuous bilirubin/ascorbic acid sensor configurations and/or continuous ketone sensor configurations and/or continuous choline sensor configurations and/or continuous glycerol sensor configurations and/or continuous creatinine sensor configurations and/or continuous lactose sensor configurations to provide a continuous multi-analyte sensor device as further described below. This continuous multi-analyte sensor device can further include continuous glucose monitoring capability. Other membranes can be used in the aforementioned uric acid sensor configuration, such as electrode, resistance, bio-interfacing, and drug releasing membranes.

### Mediators

One or more mediators can be employed to facilitate the electrolysis of one or more analytes or of a second compound that correlates with or interferes with the signal transduction of the one or more analytes. Non-polymeric and polymeric redox mediators can be used in the presently disclosed continuous multi-analyte sensor device.

In one example, zwitterionic compounds/polymers, Prussian blue, medola blue, methylene blue, methylene green, methyl viologen, ferrocyanide, ferrocene, cobalt ion and cobalt phthalocyanine can be used as a coating on one or more WEs to facilitate or otherwise assist in electron transfer and transduction of a detectable signal corresponding to one or more analytes. In one example, a transition metal complex is attached to one or more polymeric backbones as a redox mediator. In one example, the transition metal complexes include at least one substituted or unsubstituted biimidazole ligand. In another example, the transition metal complexes include at least one substituted or unsubstituted biimidazole ligand and a substituted or unsubstituted bipyridine or pyridylimidazole ligand.

In one example the mediator is one or more metal compounds or metal complexes of ruthenium, osmium, iron (e.g., polyvinylferrocene or hexacyanoferrate), or cobalt, including metallocene compounds thereof, for example. In one example, the mediator is coupled or otherwise bound to the conductive material of any one of the reference or working or counter electrode. In one example, non-polymeric or polymeric mediator can be adsorbed on or covalently bound to the conductive material of the electrode, such as a carbon surface or surfaces of gold, platinum, palladium, rhodium and alloys thereof. In one example, the mediator is quaternized.

A variety of methods may be used to immobilize a polymeric or non-polymeric mediator on an electrode surface, for example, adsorptive immobilization with or without cross-linking, vapor depositing, functionalization of at least a portion of the electrode surface and then chemical bonding, (ionically or covalently), of the mediator polymer to the functional groups on the electrode surface. In one example, poly(4-vinylpyridine) or poly vinylpyridine-co-styrene or polyvinylimidazoles are at least in part complexed with a transition metal compound, such as [Os(bpy)2 Cl]^{+/2+} where bpy is 2,2′-bipyridine. In one example, at least a part of the pyridine rings of the poly(4-vinylpyridine) or poly vinylpyridine-co-styrene are reacted with 2-bromoethylamine, then crosslinked, for example, using a diepoxide, such as polyethylene glycol diglycidyl ether. Other polymeric and/or non-polymeric mediators can be used, such as PVI- and PVP-Ruthenium (phenanthroline dione).

Carbon surfaces can be modified for attachment of one or more polymeric and/or non-polymeric mediators, for example, by electroreduction of a diazonium salt, followed by activated by a carbodiimide, such as 1-ethyl-3-(3-dimethylaminopropyl)-carbodiimide hydrochloride then bound with a amine-functionalized mediator, such as the osmium-containing polymer described above, or 2-aminoethylferrocene, to form the mediator couple.

Similarly, gold can be functionalized by a thiol or an amine, such as cysteamine and mediator [Os(bpy)2 (pyridine-4-carboxylate)Cl]0/+ can be activated by 1-ethyl-3-(3-dimethylaminopropyl)-carbodiimide hydrochloride to form a reactive O-acylisourea that reacts with the gold-bound amine to form an amide.

### Modified Enzymes

In other examples, a genetic variant of any one of the aforementioned enzymes is used, for example, a variant that improves thermal resistance, e.g., storage or shelf stability and/or operational stability. Examples of genetic mutation to improve enzyme thermal stability include, but are not limited to addition of stabilizers, such as substrates and similar ligands, sugars, polymers, specific and non-specific ion species and small uncharged organic molecules, immobilization, protein engineering (e.g., site directed mutagenesis), and/or chemical modification. In one example, isolated enzymes from anaerobic extreme thermophiles, such as NADH oxidase isolated from Clostridium thermohydrosulfuricum, Thermus thermophilus, Thermoanaerobium brockii, Streptococcus mutans, Pyrococcus horikoshii, Bacillus licheniformis are used to impart at least some thermal operational stability, e.g. up to about 80° C., to the sensor.

### Additional Mono- and Multi-Electrode Configurations

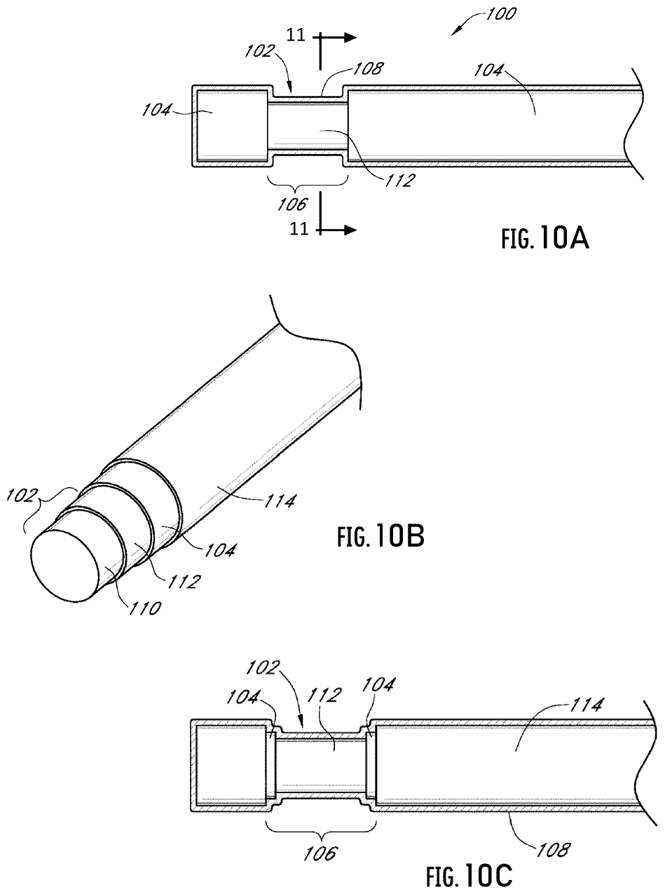

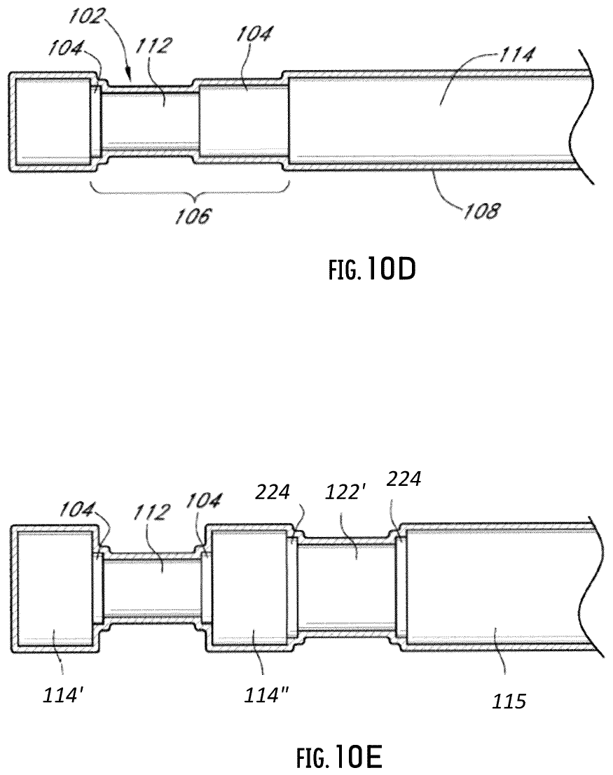

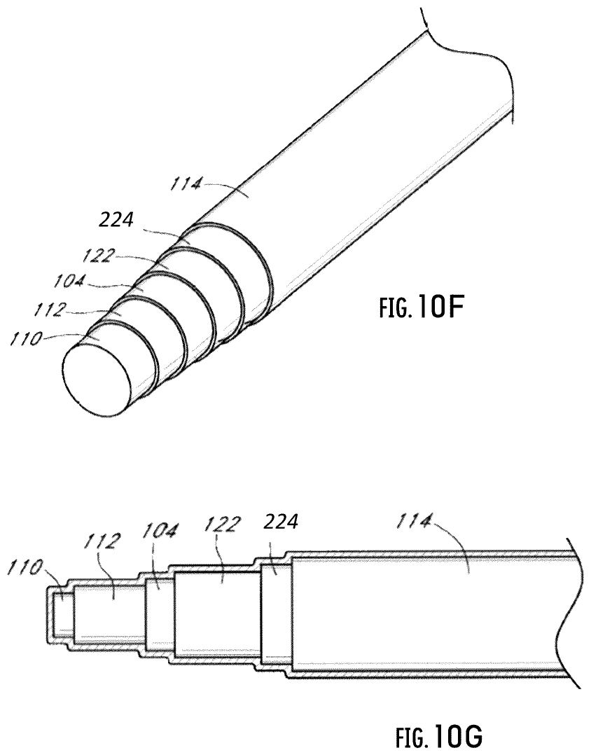

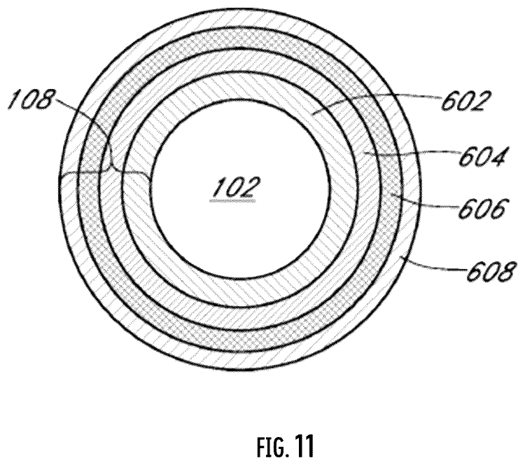

The aforementioned multi-analyte sensor configurations can be adapted to continuous multi-analyte sensor electrode configurations. For example, FIGS. 10A through 10C illustrate one aspect (e.g., the in vivo portion) of a continuous multi-analyte sensor 100, which includes an elongated conductive body 102. The elongated conductive body 102 includes a core 110 (see FIG. 10B) and a first conductive layer 112 at least partially surrounding the core. The first layer includes a working electrode (e.g., located in window 106) and a membrane 108 located over the working electrode configured and arranged for multi-axis bending. In some examples, the core and first layer can be of a single material (e.g., platinum). In some examples, the elongated conductive body is a composite of at least two materials, such as a composite of two conductive materials, or a composite of at least one conductive material and at least one non-conductive material. In some examples, the elongated conductive body comprises a plurality of layers. In certain examples, there are at least two concentric (e.g., annular) layers, such as a core formed of a first material and a first layer formed of a second material. However, additional layers can be included in some examples. In some examples, the layers are coaxial.

The elongated conductive body may be long and thin, yet flexible and strong. For example, in some examples, the smallest dimension of the elongated conductive body is less than about 0.1 inches, 0.75 inches, 0.05 inches, 0.25 inches, 0.01 inches, 0.004 inches, or 0.002 inches. While the elongated conductive body is illustrated in FIGS. 10A through 10C as having a circular cross-section, in other examples the cross-section of the elongated conductive body can be ovoid, rectangular, triangular, polyhedral, star-shaped, C-shaped, T-shaped, X-shaped, Y-shaped, irregular, or the like. In one example, a conductive wire electrode is employed as a core. To such a clad electrode, two additional conducting layers may be added (e.g., with intervening insulating layers provided for electrical isolation). The conductive layers can be comprised of any suitable material. In certain examples, it can be desirable to employ a conductive layer comprising conductive particles (i.e., particles of a conductive material) in a polymer or other binder.

In one example, the materials used to form the elongated conductive body (e.g., stainless steel, titanium, tantalum, platinum, platinum-iridium, iridium, certain polymers, and/or the like) can be strong and hard, and therefore are resistant to breakage. For example, in some examples, the ultimate tensile strength of the elongated conductive body is from about 80 kPsi to about 500 kPsi. In another example, in some examples, the Young's modulus of the elongated conductive body is from about 160 GPa to about 220 GPa. In still another example, in some examples, the yield strength of the elongated conductive body is from about 60 kPsi to about 2200 MPa. Ultimate tensile strength, Young's modulus, and yield strength are discussed in greater detail elsewhere herein. In some examples, the sensor's small diameter provides (e.g., imparts, enables) flexibility to these materials, and therefore to the sensor as a whole.

FIG. 10B is a perspective-view schematic illustrating an in vivo portion of an analyte sensor, in one example. In some examples, the sensor further comprises a third layer 114 comprising a conductive material. In further examples, the third layer 114 may comprise a reference electrode, which may be formed of a silver-containing material that is applied onto the second layer 104 (e.g., an insulator). The silver-containing material may include any of a variety of materials and be in various forms, such as, Ag/AgCl-polymer pastes, paints, polymer-based conducting mixture, and/or inks that are commercially available, for example. The third layer 114 can be processed using a pasting/dipping/coating step, for example, using a die-metered dip-coating process. In one exemplary example, an Ag/AgCl polymer paste is applied to an elongated body by dip-coating the body (e.g., using a meniscus coating technique) and then drawing the body through a die to meter the coating to a precise thickness. In some examples, multiple coating steps are used to build up the coating to a predetermined thickness. In one example, the third (or additional layer(s)) layer 114 comprises one or more resistance, bio-interfacing, and drug releasing membranes.

FIG. 10C is a side-view schematic illustrating an in vivo portion of a continuous multi-analyte sensor. As previously described with reference to FIG. 10A and as shown in FIG. 10C, an insulator 104 is disposed on (e.g., located on, covers) at least a portion of the elongated conductive body 102. In some examples, the sensor is configured and arranged such that the elongated body includes a core 110 and a first conductive layer 112, and a portion of the first conductive layer 112 is exposed via window 106 in the insulator 104. In other examples, the sensor is configured and arranged such that the elongated conductive body 102 includes a core embedded in an insulator, and a portion of the core is exposed via the window in the insulator. For example, in some examples, the insulating material is applied to the elongated conductive body 102 (e.g., screen-, dipping, ink-jet and/or block-printing) in a configuration designed to leave a portion of the surface of the first conductive layer 112 (or the surface of the core 110) exposed. For example, the insulating material used for insulator 104 other insulating layers discussed herein can be formed in a pattern that does not cover a portion of the elongated body. In another example, a portion of the elongated body is masked prior to application of the insulating material. Removal of the mask, after insulating material application, exposes the portion of the elongated body. In another example, a series of conductive and insulating layer may be plated, dipped, or otherwise formed.

In some examples, the insulator 104 is applied as a single layer of material. In other examples, the insulator 104 is applied as two or more layers, which are comprised of either the same or different materials. In some examples, the insulating material comprises at least one of polyurethane, polyimide and parylene. In one example, the insulating material comprises parylene, which can be an advantageous polymer coating for its strength, lubricity, and electrical insulation properties. Generally, parylene is produced by vapor deposition and polymerization of para-xylylene (or its substituted derivatives). However, any suitable insulating material, such as but not limited to a dielectric ink, paste or paint, can be used, for example, fluorinated polymers, polyethyleneterephthalate (PET), polyurethane, polyimide, other nonconducting polymers, or the like.

In some examples, the insulator 104 comprises a polymer, for example, a non-conductive (e.g., dielectric) polymer. Dip-coating, spray-coating, vapor-deposition, printing and/or other thin film and/or thick film coating or deposition techniques can be used to deposit the insulating material on the elongated body and/or core.

FIGS. 10C and 10D illustrate the results of this removal/cutting away process through a side-view/cross-section. The removal process can be accomplished by various methods. In one example the removal step is conducted, e.g., by laser skiving, and can be performed in a reel-to-reel process on a continuous strand. The removed area can be stepped, for example, by removing different layers by different lengths (FIG. 10D). In such a fabrication method involving a continuous strand, the sensors can be singularized after the removal step. In some examples, if the core is a metal, an end cap may be employed, e.g., by dipping, spraying, shrink tubing, crimp wrapping, etc., an insulating or other isolating material onto the tip. If the core is a polymer (e.g., hydrophobic material), an end cap may not be necessary. For example, in the sensor depicted in FIG. 10D, an end cap (e.g., of a polymer or an insulating material) or other structure may be provided over the core (e.g., if the core 110 is not insulating).

FIG. 10F can be considered to build on a general structure as depicted in FIG. 10B, in that two or more additional layers are added to create one or more additional electrodes. Methods for selectively removing two or more windows to create two or more electrodes can also be employed. For example, by adding another conductive layer 122′ and insulating layer 224 over a reference electrode domain 115, then two electrodes (first and second working electrodes 114′ and 114″) can be formed, yielding a multielectrode sensor. The same concept can be applied to create, a counter electrode, electrodes to measure additional analytes (e.g., oxygen), and the like, for example.

FIG. 10G illustrates another example, wherein selective removal of the various layers is stepped to expose the working electrodes (shown as the exposed portions of 112, 122′ and insulators 104, 224 along the length of the elongated body. In one example, the dual electrode sensor can include a first working electrode (e.g., formed from the first conductive layer 112) configured to detect glucose, and a second working electrode (e.g., formed from the second conductive layer 122′), configured to detect lactate. In one example, the dual electrode sensor can include a first working electrode (e.g., formed from the first conductive layer 112) configured to detect glucose, and a second working electrode (e.g., formed from the second conductive layer 122′), configured to detect ketone. In another example, both working electrodes are configured to detect the same analyte.

FIG. 11 is a cross-sectional schematic of an exemplary membrane configuration of the multi-analyte sensor of FIG. 10A, taken on line 11-11. In one example, the membrane system includes a plurality of domains, for example, an electrode domain 602, an interference domain 304, a transducing element domain 606 (for example, enzyme, aptamer, ionophore, etc.), and/or a resistance domain 608, as shown in FIG. 11, and can include a high oxygen solubility domain, and/or a bioprotective domain and/or drug releasing domain (not shown), such as is described in more detail in U.S. Patent Application Publication No. US-2005-0245799-A1, and such. The membrane system(s) can be independently deposited on the exposed electroactive surfaces using known thin film techniques (for example, vapor deposition, spraying, electro-depositing, dipping, and the like).

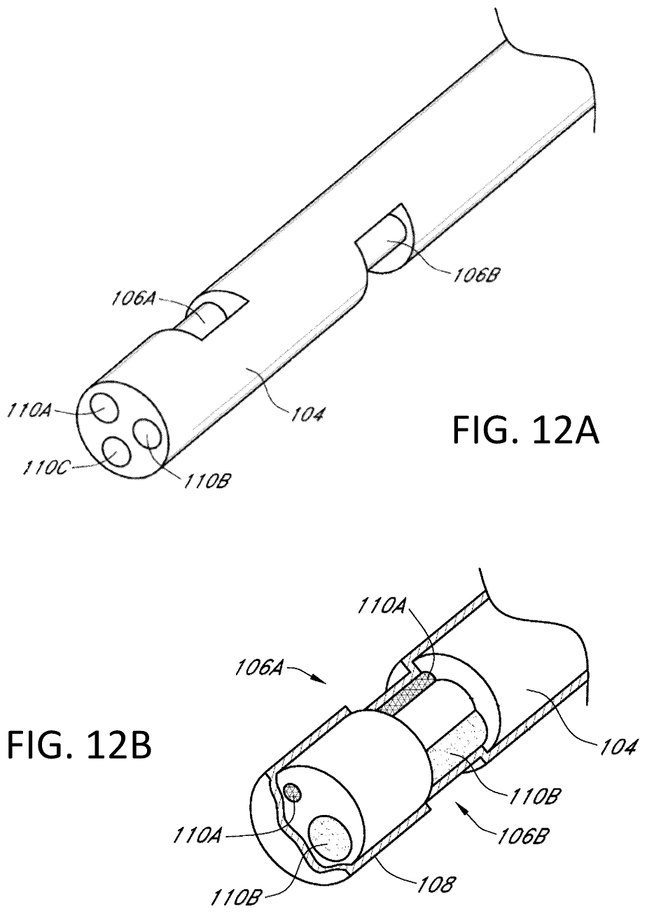

FIG. 12A is a perspective view of the in vivo portion of a single electrode, dual working electrode surface multi-analyte sensor (e.g., configured and arranged for multi-axis bending). In this example, the insulated elongated body comprises three conductive cores 110A, 1106, 110C located in (e.g., embedded in, coated with) the insulator 104. In this example, a plurality of windows is formed in and/or through the insulator, such that each window exposes a portion of a core. As a non-limiting example, window 106A is formed in the insulator such that a portion of core 110A is exposed. Similarly, window 106B is formed in the insulator such that a portion of core 1106 is exposed. The windows can be staggered and/or non-staggered along the longitudinal length of the sensor. In a further example, each conductive core includes an inner core and an outer core, such as described elsewhere herein.

FIG. 12B is a perspective view of the in vivo portion of an analyte sensor including an elongated body (e.g., configured and arranged for multi-axis bending) formed of an insulator 104, first and second conductive cores 110A, 110B embedded in the insulator, and a membrane 108. The first conductive core is formed of platinum, platinum-iridium, gold, palladium, iridium, graphite, carbon, a conductive polymer and/or an alloy, and a first window 106A is configured and arranged to expose an electroactive portion of the first conductive core. The second conductive core is formed of a silver-containing material (e.g., a silver or silver/silver-chloride wire, or a silver-containing wire-shaped a silver-containing material body), and a second window 106B is configured and arranged to expose an electroactive portion of the second conductive core. In some examples, instead of a bulk metal wire, the first conductive core comprises an inner core and an outer core. For example, to reduce material costs, the inner core is formed of a material that is relatively less expensive than platinum, such as stainless steel, titanium, tantalum and/or a polymer, and the outer core is formed of a material that provides an appropriate electroactive surface, such as but not limited to platinum, platinum-iridium, gold, palladium, iridium, graphite, carbon, a conductive polymer and/or an alloy. In some examples, the membrane covers the exposed electroactive portion of the first conductive core. In a further example, the membrane covers the in vivo portion of the sensor. In some examples, a third conductive core is embedded in the insulator. In some examples, the third conductive core is configured and arranged as a second working electrode, which can be configured as a redundant working electrode, a non-analyte signal-measuring working electrode (e.g., no transducing element as described below), as a counter working electrode, to detect a second analyte, and/or the like.

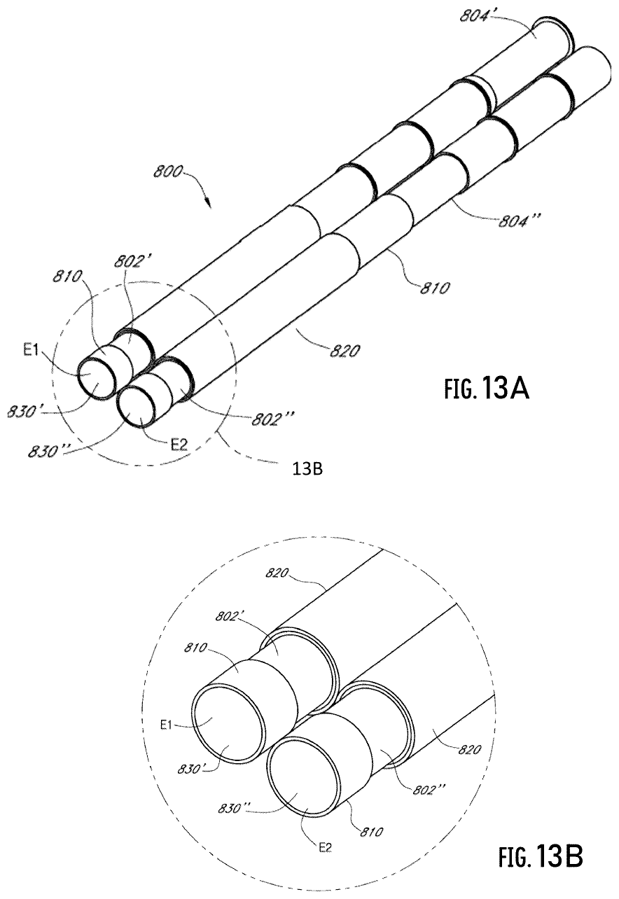

FIG. 13A is a perspective view of the in vivo portion of another example of a multi-electrode sensor system 800 comprising two working electrodes and at least one reference/counter electrode. The sensor system 800 comprises first and second elongated bodies E1, E2, each formed of a conductive core or of a core with a conductive layer deposited thereon. In this particular example, an insulating layer 810, a conductive layer 820, and any one of the previously described membranes (not shown) are deposited on top of the elongated bodies E1, E2. The insulating layer 810 separates the conductive layer 820 from the elongated body. The materials selected to form the insulating layer 810 may include any of the insulating materials described elsewhere herein, including polyurethane and polyimide. The materials selected to form the conductive layer 820 may include any of the conductive materials described elsewhere herein, including silver/silver chloride, platinum, gold, etc. Working electrodes 802′, 802″ are formed by removing portions of the conductive layer 820 and the insulating layer 810, thereby exposing electroactive surface of the elongated bodies E1, E2, respectively. FIG. 13B provides a close perspective view of the distal portion of the elongated bodies E1, E2.

In one example, the two elongated bodies illustrated in FIG. 13A are fabricated to have substantially the same shape and dimensions. In some examples, the working electrodes are fabricated to have the same properties, thereby providing a sensor system capable of providing redundancy of signal measurements or providing unique signals representing two or more different analytes. In other examples, the working electrodes, associated with the elongated bodies E1, E2, may each have one or more characteristics that distinguish each working electrode from the other. For example, in one example, each of the elongated bodies E1, E2 may be different conductive surfaces, so that each working electrode has a different electrochemical property than the other working electrode. In addition, in one example, each of the elongated bodies E1, E2 may be covered with different membrane(s), so that each working electrode has a different membrane property than the other working electrode. For example, one of the working electrodes may have a membrane comprising a first transducing element and the other working electrode may have a membrane comprising a layer having either an inactivated form of the transducing element, or no transducing element, aptamer(s), or cofactor(s). Additional sensor system configurations that are possible with a plurality of working electrodes (e.g., sensor elements) are described in U.S. Provisional Application No. 61/222,716 filed Jul. 2, 2009 and U.S. patent application Ser. No. 12/829,264, filed Jul. 1, 2010, entitled “ANALYTE SENSOR,” each of which is incorporated by reference herein in its entirety.

Although not shown in FIGS. 13A-13B, in certain examples, the distal ends 830′, 830″ of the core portions of the elongated bodies E1, E2 may be covered with an insulating material (e.g., polyurethane or polyimide). In alternative examples, the exposed core portions 830′, 830″ may be covered with any of the previously described membrane system and/or serve as additional working electrode surface area.

Regarding fabrication of the sensor system illustrated in FIG. 13A-13B, in one example, the elongated bodies E1, E2 may be formed as an elongated conductive core, or alternatively as a core (conductive or non-conductive) having at least one conductive material deposited thereon. Next, an insulating layer 810 is deposited onto each of the elongated bodies E1, E2. Thereafter, a conductive layer 820 is deposited over the insulating layer 810. The conductive layer 820 may serve as a reference/counter electrode and may be formed of silver/silver chloride, or any other material that may be used for a reference electrode. In alternative examples, the conductive layer 820 may be formed of a different conductive material, and may be used another working electrode. After these steps, a layer removal process is performed to remove portions of the deposited layers (i.e., the conductive layer 820 and/or the insulating layer 810). Any of the techniques described elsewhere herein (e.g., laser ablation, chemical etching, grit blasting) may be used. In the example illustrated in FIGS. 13A and 13B, layers of the conductive layer 820 and the insulating layer 810 are removed to form the working electrodes 802′, 802″. Although in the example shown, layer removal is performed across the entire cross-sectional perimeter (e.g., circumference) of the deposited layer, it is contemplated that in other examples, layer removal may be performed across a preselected section of the cross-sectional perimeter, instead of across the entire cross-sectional perimeter.

Contacts 804′, 804″ used to provide electrical connection between the working electrodes and other components of the sensor system may be formed in a similar manner. As shown, contacts 804′ and 804″ are separated from each other to prevent an electrical connection therebetween. Because the layer removal process is performed on each individual elongated body E1, E2, instead of a single geometrically complicated elongated body, this particular sensor design (i.e., two elongated bodies placed side by side) may provide ease of manufacturing, as compared to the manufacturing processes involved with other multi-electrode systems having other geometries.

After the conductive and insulating layers are deposited onto the elongated body, and after selected portions of the deposited layers have been removed, a membrane is applied onto at least a portion of the elongated bodies. In certain examples, any of the aforementioned membrane systems are applied only to the working electrodes, but in other examples any of the aforementioned membrane systems are applied to the entire elongated body. In one example, any of the aforementioned membrane systems are deposited onto the two working electrodes simultaneously while they are placed together (e.g., by bundling), but in another example, any of the aforementioned membrane systems are deposited onto each individual working electrode first, and the two working electrodes are then placed together. Accordingly, in one example, any of the aforementioned membrane systems are designed with a sensitivity of from about 1 pA/mg/dL to about 100 pA/mg/dL. In other examples, the sensitivity is from about 5 pA/mg/dL to 25 pA/mg/dL. In further examples, the sensitivity is from about 4 to about 7 pA/mg/dL. While not wishing to be bound by any particular theory, it is believed that membrane systems designed with a sensitivity in the above ranges permit measurement, independently, of the one or more analyte signals in low analyte and/or low reactant/co-reactant situations. Reduced measurement accuracy in low analyte ranges may be a problem due to lower availability of the analyte to the sensor and/or have shown increased signal noise in high analyte ranges due to insufficient reactant/co-reactant necessary to react with the amount of analyte being measured. Accordingly while not wishing to be bound by theory, it is believed that the membrane systems of the present disclosure, in combination with the electronic circuitry design and exposed electrochemical reactive surface area design, support measurement of the analyte in the picoAmp range, which enables an improved level of resolution and accuracy in both low and high analyte ranges.

### Biointerface Membrane/Layer

In one example, the sensor includes a porous material disposed over some portion thereof, which modifies the host's tissue response to the sensor. In some examples, the porous material surrounding the sensor advantageously enhances and extends sensor performance and lifetime in the short term by slowing or reducing cellular migration to the sensor and associated degradation that would otherwise be caused by cellular invasion if the sensor were directly exposed to the in vivo environment. Alternately, the porous material can provide stabilization of the sensor via tissue ingrowth into the porous material in the long term. Suitable porous materials include silicone, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), polybutylene terephthalate (PBT), polymethylmethacrylate (PMMA), polyether ether ketone (PEEK), polyamides, polyurethanes, cellulosic polymers, poly(ethylene oxide), poly(propylene oxide) and copolymers and blends thereof, polysulfones and block copolymers thereof including, for example, di-block, tri-block, alternating, random and graft copolymers, as well as metals, ceramics, cellulose, hydrogel polymers, poly(2-hydroxyethyl methacrylate, pHEMA), hydroxyethyl methacrylate, (HEMA), polyacrylonitrile-polyvinyl chloride (PAN-PVC), high density polyethylene, acrylic copolymers, nylon, polyvinyl difluoride, polyanhydrides, poly(I-lysine), poly(L-lactic acid), hydroxyethylmetharcrylate, hydroxyapeptite, alumina, zirconia, carbon fiber, aluminum, calcium phosphate, titanium, titanium alloy, nitinol, stainless steel, and CoCr alloy, or the like, such as are described in U.S. Pat. No. 7,875,293 to Shults et al. and U.S. Pat. No. 7,192,450 to Brauker et al.

In some examples, the porous material surrounding the sensor provides unique advantages that can be used to enhance and extend both sensor performance and lifetime. However, such materials can also provide advantages over a period of time as well (e.g., for sensor wearability for terms of equal to or greater than 14, 15, or 21 days). Particularly, the in vivo portion of the sensor (the portion of the sensor that is implanted into the host's tissue) is encased (partially or fully) in a porous material. The porous material can be wrapped around the sensor (for example, by wrapping the porous material around the sensor or by inserting the sensor into a section of porous material sized to receive the sensor). Alternately, the porous material can be deposited on the sensor (for example, by electrospinning of a polymer directly thereon). In yet other alternate examples, the sensor is inserted into a selected section of porous biomaterial. Other methods for surrounding the in vivo portion of the sensor with a porous material can also be used as is appreciated by a person of ordinary skill in the art.

The porous material surrounding the sensor advantageously slows or reduces cellular migration to the sensor and associated degradation that would otherwise be caused by cellular invasion if the sensor were directly exposed to the in vivo environment. Namely, the porous material provides a barrier that makes the migration of cells towards the sensor more tortuous and therefore slower (providing longevity of use advantages). It is believed that this reduces or slows the sensitivity loss normally observed in a sensor over time.

In an example wherein the porous material is a high oxygen solubility material, such as porous silicone, the high oxygen solubility porous material surrounds some of or the entire in vivo portion of the sensor. In some examples, a lower ratio of oxygen-to-glucose can be sufficient to provide excess oxygen by using a high oxygen soluble domain (for example, a silicone- or fluorocarbon-based material) to enhance the supply/transport of oxygen to the enzyme domain and/or electroactive surfaces. In some examples, some signal noise normally seen by a sensor can be attributed to an oxygen deficit. Silicone has high oxygen permeability, thus promoting oxygen transport to the enzyme domain. By enhancing the oxygen supply through the use of a silicone composition, for example, glucose concentration can be less of a limiting factor. In other words, if more oxygen is supplied to the enzyme and/or electroactive surfaces, then more glucose can also be supplied to the enzyme without creating an oxygen rate-limiting excess. While not being bound by any particular theory, it is believed that silicone materials provide enhanced bio-stability when compared to other polymeric materials such as polyurethane.

In another example, the porous material further comprises a bioactive agent that releases upon insertion. In one example, the porous structure provides access for glucose permeation while allowing drug release/elution. In one example, as the bioactive agent releases/elutes from the porous structure, glucose transport may increase, for example, so as to offset any attenuation of glucose transport from the aforementioned immune response factors.

In these examples, the aforementioned porous material is a biointerface membrane comprising a first domain that includes an architecture, including cavity size, configuration, and/or overall thickness, that modifies the host's tissue response, for example, by creating a fluid pocket, encouraging vascularized tissue ingrowth, disrupting downward tissue contracture, resisting fibrous tissue growth adjacent to the device, and/or discouraging barrier cell formation. The biointerface membrane in one example covers at least the sensing mechanism of the sensor and can be of any shape or size, including uniform, asymmetrically, or axi-symmetrically covering or surrounding a sensing mechanism or sensor.

A second domain of the biointerface membrane is optionally provided that is impermeable to cells and/or cell processes. A bioactive agent is optionally provided that is incorporated into the at least one of the first domain, the second domain, the sensing membrane, or other part of the implantable device, wherein the bioactive agent is configured to modify a host tissue response. In one example, the biointerface membrane includes a bioactive agent, the bioactive agent being incorporated into at least one of the first and second domains of the biointerface membrane, or into the device and adapted to diffuse through the first and/or second domains, in order to modify the tissue response of the host to the sensor implant.

Due to the small dimension(s) of the sensor (sensing mechanism) of the present disclosure, some conventional methods of porous membrane formation and/or porous membrane adhesion are inappropriate for the formation of the biointerface membrane onto the sensor as described herein. Accordingly, the following examples exemplify systems and methods for forming and/or adhering a biointerface membrane onto a small structured sensor as defined herein. For example, the biointerface membrane or release membrane of the present disclosure can be formed onto the sensor using techniques such as electrospinning, molding, weaving, direct-writing, lyophilizing, wrapping, and the like.

In examples wherein the biointerface membrane is directly-written onto the sensor, a dispenser dispenses a polymer solution using a nozzle with a valve, or the like, for example as described in U.S. Pat. No. 7,857,756. In general, a variety of nozzles and/or dispensers can be used to dispense a polymeric material to form the woven or non-woven fibers of the biointerface membrane.

### Drug Releasing Domain—Inflammatory Response Control

A drug releasing domain that may include at least one membrane having one or more layers may be included in the membrane systems discussed herein. In general, the inflammatory response to biomaterial implants can be divided into two phases. The first phase consists of mobilization of mast cells and then infiltration of predominantly polymorphonuclear (PMN) cells. This phase is termed the acute inflammatory phase. Over the course of days to weeks, chronic cell types that comprise the second phase of inflammation replace the PMNs. Macrophage and lymphocyte cells predominate during this phase. While not wishing to be bound by any particular theory, it is believed that restricting vasodilation and/or blocking pro-inflammatory signaling, stimulation of vascularization, or inhibition of scar formation or barrier cell layer formation, provides protection from scar tissue formation and/or reduces acute inflammation, thereby providing a stable platform for sustained maintenance of the altered foreign body response, for example.

Accordingly, bioactive intervention can modify the foreign body response in the early weeks of foreign body capsule formation and alter the long-term behavior of the foreign body capsule. Additionally, it is believed that in some circumstances the biointerface membranes and/or drug releasing membranes of the present disclosure can benefit from bioactive intervention to overcome sensitivity of the biointerface membrane and/or drug releasing membrane to implant procedure, motion of the implant, or other factors, which are known to otherwise cause inflammation, scar formation, and hinder device function in vivo.

In general, bioactive agents that are believed to modify tissue response include anti-inflammatory agents, anti-infective agents, anti-proliferative agents, anti-histamine agents, anesthetics, inflammatory agents, growth factors, angiogenic (growth) factors, adjuvants, immunosuppressive agents, antiplatelet agents, anticoagulants, ACE inhibitors, cytotoxic agents, anti-barrier cell compounds, vascularization compounds, anti-sense molecules, and the like. In some examples, bioactive agents include S1P (Sphingosine-1-phosphate), Monobutyrin, Cyclosporin A, Anti-thrombospondin-2, Rapamycin (and its derivatives), and Dexamethasone. However, other bioactive agents, biological materials (for example, proteins), or even non-bioactive substances can be incorporated into the biointerface membranes and/or drug releasing membranes of the present disclosure.

Bioactive agents suitable for use in the present disclosure are loosely organized into two groups: anti-barrier cell agents and vascularization agents. These designations reflect functions that are believed to provide short-term solute transport through the one or more membranes of the presently disclosed sensor, and additionally extend the life of a healthy vascular bed and hence solute transport through the one or more membranes long term in vivo. However, not all bioactive agents can be clearly categorized into one or other of the above groups; rather, bioactive agents generally comprise one or more varying mechanisms for modifying tissue response and can be generally categorized into one or both of the above-cited categories.

### Anti-Barrier Cell Agents

Generally, anti-barrier cell agents include compounds exhibiting effects on macrophages and foreign body giant cells (FBGCs). It is believed that anti-barrier cell agents prevent closure of the barrier to solute transport presented by macrophages and FBGCs at the device-tissue interface during FBC maturation.

Anti-barrier cell agents generally include mechanisms that inhibit foreign body giant cells and/or occlusive cell layers. For example, Super Oxide Dismutase (SOD) Mimetic, which utilizes a manganese catalytic center within a porphyrin like molecule to mimic native SOD and effectively remove superoxide for long periods, thereby inhibiting FBGC formation at the surfaces of biomaterials in vivo, is incorporated into a biointerface membrane or release membrane.

Anti-barrier cell agents can include nano- or micro structures, anti-inflammatory and/or immunosuppressive mechanisms that affect early FBC formation. Cyclosporine, which stimulates very high levels of neovascularization around biomaterials, can be incorporated into a biointerface membrane (see U.S. Pat. No. 5,569,462 to Martinson et al.), or release membrane.

In one example, dexamethasone, dexamethasone salts, or dexamethasone derivatives in particular, dexamethasone acetate, which, for example, abates the intensity of the FBC response at the device-tissue interface, is incorporated into the drug releasing membrane. In another example, a combination of dexamethasone and dexamethasone acetate is incorporated into the drug releasing membrane. In another example, dexamethasone and/or dexamethasone acetate combined with one or more other anti-inflammatory and/or immunosuppressive agents is incorporated into the drug releasing membrane. Alternately, Rapamycin, which is a potent specific inhibitor of some macrophage inflammatory functions, can be incorporated into the release membrane alone or in combination with dexamethasone, dexamethasone salts, or dexamethasone derivatives, in particular, dexamethasone acetate.

Other suitable medicaments, pharmaceutical compositions, therapeutic agents, or other desirable substances can be incorporated into the drug releasing membrane of the present disclosure, including, but not limited to, anti-inflammatory agents, anti-infective agents, necrosing agents, and anesthetics.

Generally, anti-inflammatory agents reduce acute and/or chronic inflammation adjacent to the implant, in order to decrease the formation of an FBC to reduce or prevent barrier cell layer formation. Suitable anti-inflammatory agents include but are not limited to, for example, nonsteroidal anti-inflammatory drugs (NSAIDs) such as acetaminophen, aminosalicylic acid, aspirin, celecoxib, choline magnesium trisalicylate, diclofenac potassium, diclofenac sodium, diflunisal, etodolac, fenoprofen, flurbiprofen, ibuprofen, indomethacin, interleukin (IL)-10, IL-6 mutein, anti-IL-6 iNOS inhibitors (for example, L-NAME or L-NMDA), Interferon, ketoprofen, ketorolac, leflunomide, melenamic acid, mycophenolic acid, mizoribine, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, paclitaxel, tacrolimus, tranilast, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, dexamethasone, dexamethasone salts, dexamethasone derivatives, and dexamethasone acetate.

Generally, immunosuppressive and/or immunomodulatory agents interfere directly with several key mechanisms necessary for involvement of different cellular elements in the inflammatory response. Suitable immunosuppressive and/or immunomodulatory agents include anti-proliferative, cell-cycle inhibitors, (for example, paclitaxol (e.g., Sirolimus), cytochalasin D, infiximab), taxol, actinomycin, mitomycin, thospromote VEGF, estradiols, NO donors, QP-2, tacrolimus, tranilast, actinomycin, everolim us, methothrexate, mycophenolic acid, angiopeptin, vincristing, mitomycine, statins, C MYC antisense, sirolimus (and analogs), RestenASE, 2-chloro-deoxyadenosine, PCNA Ribozyme, batimstat, prolyl hydroxylase inhibitors, PPARy ligands (for example troglitazone, rosiglitazone, pioglitazone), halofuginone, C-proteinase inhibitors, probucol, BCP671, EPC antibodies, catchins, glycating agents, endothelin inhibitors (for example, Ambrisentan, Tesosentan, Bosentan), Statins (for example, Cerivasttin), E. coli heat-labile enterotoxin, and advanced coatings.

Generally, anti-infective agents are substances capable of acting against infection by inhibiting the spread of an infectious agent or by killing the infectious agent outright, which can serve to reduce immuno-response without inflammatory response at the implant site. Anti-infective agents include, but are not limited to, anthelmintics (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin; clindamycin; colistimethate sodium; polymyxin b sulfate; vancomycin; antivirals including acyclovir, amantadine, didanosine, efavirenz, foscarnet, ganciclovir, indinavir, lamivudine, nelfinavir, ritonavir, saquinavir, silver, stavudine, valacyclovir, valganciclovir, zidovudine; quinolones (ciprofloxacin, levofloxacin); sulfonamides (sulfadiazine, sulfisoxazole); sulfones (dapsone); furazolidone; metronidazole; pentamidine; sulfanilamidum crystallinum; gatifloxacin; and sulfamethoxazole/trimethoprim.

Generally, necrosing agents are any drug that causes tissue necrosis or cell death. Necrosing agents include cisplatin, BCNU, taxol or taxol derivatives, and the like.

### Vascularization Agents

Generally, vascularization agents include substances with direct or indirect angiogenic properties. In some cases, vascularization agents may additionally affect formation of barrier cells in vivo. By indirect angiogenesis, it is meant that the angiogenesis can be mediated through inflammatory or immune stimulatory pathways. It is not fully known how agents that induce local vascularization indirectly inhibit barrier-cell formation; however, it is believed that some barrier-cell effects can result indirectly from the effects of vascularization agents.

Vascularization agents include mechanisms that promote neovascularization around the sensing membrane and/or minimize periods of ischemia by increasing vascularization close to the device-tissue interface. Sphingosine-1-Phosphate (S1P), which is a phospholipid possessing potent angiogenic activity, is incorporated into a biointerface membrane or release membrane. Monobutyrin, which is a potent vasodilator and angiogenic lipid product of adipocytes, is incorporated into a biointerface membrane or release membrane. In another example, an anti-sense molecule (for example, thrombospondin-2 anti-sense), which increases vascularization, is incorporated into a biointerface membrane or release membrane.

Vascularization agents can include mechanisms that promote inflammation, which is believed to cause accelerated neovascularization in vivo. In one example, a xenogenic carrier, for example, bovine collagen, which by its foreign nature invokes an immune response, stimulates neovascularization, and is incorporated into a biointerface membrane or release membrane of the present disclosure. In another example, Lipopolysaccharide, which is a potent immunostimulant, is incorporated into a biointerface membrane or release membrane. In another example, a protein, for example, a bone morphogenetic protein (BMP), which is known to modulate bone healing in tissue, is incorporated into a biointerface membrane or release membrane.

Generally, angiogenic agents are substances capable of stimulating neovascularization, which can accelerate and sustain the development of a vascularized tissue bed at the device-tissue interface. Angiogenic agents include, but are not limited to, copper ions, iron ions, tridodecylmethylammonium chloride, Basic Fibroblast Growth Factor (bFGF), (also known as Heparin Binding Growth Factor-II and Fibroblast Growth Factor II), Acidic Fibroblast Growth Factor (aFGF), (also known as Heparin Binding Growth Factor-I and Fibroblast Growth Factor-I), Vascular Endothelial Growth Factor (VEGF), Platelet Derived Endothelial Cell Growth Factor BB (PDEGF-BB), Angiopoietin-1, Transforming Growth Factor Beta (TGF-Beta), Transforming Growth Factor Alpha (TGF-Alpha), Hepatocyte Growth Factor, Tumor Necrosis Factor-Alpha (TNF-Alpha), Placental Growth Factor (PLGF), Angiogenin, Interleukin-8 (IL-8), Hypoxia Inducible Factor-I (HIF-1), Angiotensin-Converting Enzyme (ACE) Inhibitor Quinaprilat, Angiotropin, Thrombospondin, Peptide KGHK, Low Oxygen Tension, Lactic Acid, Insulin, Copper Sulphate, Estradiol, prostaglandins, cox inhibitors, endothelial cell binding agents (for example, decorin or vimentin), glenipin, hydrogen peroxide, nicotine, and Growth Hormone.

Generally, pro-inflammatory agents are substances capable of stimulating an immune response in host tissue, which can accelerate or sustain formation of a mature vascularized tissue bed. For example, pro-inflammatory agents are generally irritants or other substances that induce chronic inflammation and chronic granular response at the implantation-site. While not wishing to be bound by theory, it is believed that formation of high tissue granulation induces blood vessels, which supply an adequate or rich supply of analytes to the device-tissue interface. Pro-inflammatory agents include, but are not limited to, xenogenic carriers, Lipopolysaccharides, S. aureus peptidoglycan, and proteins.

The bioactive agents can be spatially distributed or dispersed throughout the drug releasing membrane where the spatial distribution or dispersion can be uniform or nonuniform, and/or vary vertically and/or horizontally in a gradient. Other substances that can be incorporated into membranes of the present disclosure include various pharmacological agents, excipients, and other substances suitable for use in pharmaceutical formulations.

Although the bioactive agent in some examples is incorporated into the biointerface membrane or release membrane and/or implantable device, in other examples the bioactive agent can be administered concurrently with, prior to, or after implantation of the device systemically, for example, by oral administration, or locally, for example, by subcutaneous injection near the implantation site, or transdermally via the adhesive used to secure the analyte sensor to the host. Thus, a depot of bioactive agent can be present in the adhesive or the analyte sensor housing for passive or post-controlled release of the bioactive agent during use of the analyte sensor.

In one example, the drug releasing membrane functions as a biointerface membrane. In another example, the drug releasing membrane is chemically distinct from the biointerface membrane. In this example, one or more bioactive agents can be incorporated into each of the biointerface membrane and the drug releasing membrane. In this example, the biointerface membrane and the drug releasing membrane may each include the same bioactive agent(s). In other examples, the biointerface membrane and the drug releasing membrane may each independently include one or more bioactive agents that differ in, for example, chemistry, loading (wt. %) of the respective membrane, or other factors or combinations of factors. In another example membrane system, a drug releasing membrane is present but no biointerface membrane is used. In such examples, one or more bioactive agents are incorporated into the drug releasing membrane. Generally, numerous variables can affect the pharmacokinetics of bioactive agent release. The bioactive agents of the present disclosure can be configured for short- and/or long-term release. In some examples, the bioactive agents of the present disclosure are designed to aid or overcome factors associated with short-term effects (for example, acute inflammation) of the foreign body response, which can begin as early as the time of implantation and extend up to about one month after implantation. In some examples, the bioactive agents of the present disclosure are designed to aid or overcome factors associated with long-term effects, for example, chronic inflammation, barrier cell layer formation, or build-up of fibrotic tissue of the foreign body response, which can begin as early as about one week after implantation and extend for the life of the implant, for example, months to years. In some examples, the bioactive agents of the present disclosure combine short- and long-term release to exploit the benefits of both. U.S. Pat. No. 7,875,293 to Shults et al., U.S. Provisional Applications 63/318,901, filed Mar. 11, 2022, U.S. patent application Ser. No. 17/697,701 discloses a variety of systems and methods for release of the bioactive agents, the discloses of which are incorporated by reference herein.

### Continuous Multianalyte Sensor

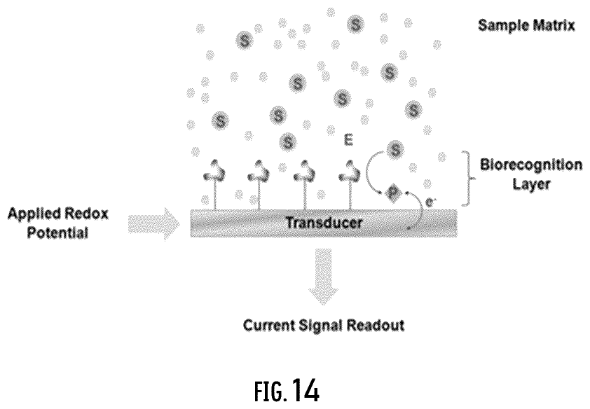

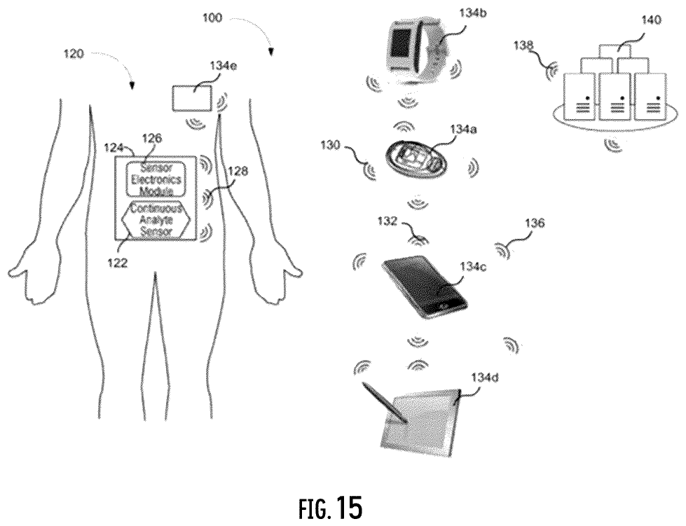

FIG. 14 shows a basic schematic of the operating principle underpinning amperometric enzymatic sensors, where S represents the substrate that is the target analyte (e.g., glucose, lactate, ketones), E represents the enzyme (e.g., glucose oxidase, lactate oxidase, 3-hydroxybutyrate dehydrogenase), and P represents the product of the enzymatic reaction that, in one example, is either reduced or oxidized at the transducer (electrode) surface by the application of a sufficient bias potential. In one example, a potentiostat, which is operably connected to an electrode system (such as described above) provides a voltage to the electrodes, which biases the sensor to enable measurement of an current signal indicative of the analyte concentration in the host (also referred to as the analog portion).

FIG. 15 is a diagram depicting an example continuous analyte monitoring system configured to measure two or more analytes and/or electrophysiological indicators (e.g., blood pressure, heart rate, core temperature, etc.) as discussed herein. The monitoring system includes a continuous sensor system 124 operatively connected to a host 120 and a plurality of display devices 134 a-e according to certain aspects of the present disclosure. It should be noted that display device 134e alternatively or in addition to being a display device, may be a medicament delivery device that can act cooperatively with the continuous analyte sensor system 124 to deliver medicaments to host 120. The continuous sensor system 124 may include a sensor electronics module 126 and a continuous analyte sensor 122 associated with the sensor electronics module 126. The sensor electronics module 126 may be in direct wireless communication with one or more of the plurality of the display devices 134a-e via wireless communications signals. In one example, display devices 134a-e may also communicate amongst each other and/or through each other to continuous analyte sensor system 124. For ease of reference, wireless communications signals from continuous analyte sensor system 124 to display devices 134a-e can be referred to as “uplink” signals 128. Wireless communications signals from, e.g., display devices 134a-e to continuous analyte sensor system 124 can be referred to as “downlink” signals 130. Wireless communication signals between two or more of display devices 134a-e may be referred to as “crosslink” signals 132. Additionally, wireless communication signals can include data transmitted by one or more of display devices 134a-d via “long-range” uplink signals 136 (e.g., cellular signals) to one or more remote servers 140 or network entities, such as cloud-based servers or databases, and receive long-range downlink signals 138 transmitted by remote servers 140.

The sensor electronics module 126 includes sensor electronics that are configured to process sensor information and generate transformed sensor information. In certain examples, the sensor electronics module 126 includes electronic circuitry associated with measuring and processing data from continuous analyte sensor 122, including prospective algorithms associated with processing and calibration of the continuous analyte sensor data. The sensor electronics module 126 can be integral with (non-releasably attached to) or releasably attachable to the continuous analyte sensor 122 achieving a physical connection therebetween. The sensor electronics module 126 may include hardware, firmware, and/or software that enables analyte level measurement. For example, the sensor electronics module 126 can include a potentiostat, a power source for providing power to continuous analyte sensor 122, other components useful for signal processing and data storage, and a telemetry module for transmitting data from itself to one or more display devices 134a-e. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, each of which are incorporated herein by reference in their entirety for all purposes.

Display devices 134a-e are configured for displaying, alarming, and/or basing medicament delivery on the sensor information that has been transmitted by the sensor electronics module 126 (e.g., in a customized data package that is transmitted to one or more of display devices 134a-e based on their respective preferences). Each of the display devices 134a-e can include a display such as a touchscreen display for displaying sensor information to a user (most often host 120 or a care taker/medical professional) and/or receiving inputs from the user. In some examples, the display devices 134a-e may include other types of user interfaces such as a voice user interface instead of or in addition to a touchscreen display for communicating sensor information to the user of the display device 134a-e and/or receiving user inputs. In some examples, one, some or all of the display devices 134a-e are configured to display or otherwise communicate the sensor information as it is communicated from the sensor electronics module 126 (e.g., in a data package that is transmitted to respective display devices 134a-e), without any additional prospective processing required for calibration and real-time display of the sensor information.

In the example of FIG. 15, one of the plurality of display devices 134a-e may be a custom display device 134a specially designed for displaying certain types of displayable sensor information associated with analyte values received from the sensor electronics module 126 (e.g., a numerical value and an arrow, in some examples). In some examples, one of the plurality of display devices 134a-e may be a handheld device 134c, such as a mobile phone based on the Android, iOS operating system or other operating system, a palm-top computer and the like, where handheld device 134c may have a relatively larger display and be configured to display a graphical representation of the continuous sensor data (e.g., including current and historic data). Other display devices can include other hand-held devices, such as a tablet 134d, a smart watch 134b, a medicament delivery device 134e, a blood glucose meter, and/or a desktop or laptop computers.

As alluded to above, because the different display devices 134a-e provide different user interfaces, content of the data packages (e.g., amount, format, and/or type of data to be displayed, alarms, and the like) can be customized (e.g., programmed differently by the manufacture and/or by an end user) for each particular display device and/or display device type. Accordingly, in the example of FIG. 15, one or more of display devices 134a-e can be in direct or indirect wireless communication with the sensor electronics module 126 to enable a plurality of different types and/or levels of display and/or functionality associated with the sensor information, which is described in more detail elsewhere herein.

Generally, continuous analyte sensor 122 may be an implantable analyte sensor that utilizes amperometric electrochemical sensor technology to measure an analyte concentration. Electrodes comprising continuous analyte sensor 122 may include a working electrode, a counter electrode, and a reference electrode. In one example, the counter electrode is provided to balance the current generated by the species being measured at the working electrode.

In some alternative examples, additional electrodes can be included within the assembly, for example, a three-electrode system (working, reference, and counter electrodes) and an additional working electrode (e.g., an electrode which can be used to generate oxygen, which is configured as a baseline subtracting electrode, or which is configured for measuring additional analytes, including but not limited to the analytes discussed herein). U.S. Pat. No. 7,081,195, U.S. Patent Publication No. 2005/0143635 and U.S. Patent Publication No. 2007/0027385, each of which are incorporated herein by reference in its entirety, describe some systems and methods for implementing and using additional working, counter, and reference electrodes.

### Sensor Electronics

The following description of electronics associated with the sensor is applicable to a variety of continuous multi-analyte sensors, such as non-invasive, minimally invasive, and/or invasive (e.g., transcutaneous and implantable) sensors. For example, the sensor electronics and data processing as well as the receiver electronics and data processing described below can be incorporated into the implantable glucose sensor disclosed in U.S. Pat. Appl. Pub. No. 2005/0245799 to Brauker et al. and U.S. Pat. Appl. Pub. No. 2006/0015020 to Neale et al.

The high-fidelity readout of analytes using electrochemical methods uses specialized hardware. Application of a stable prescribed bias potential is used since the redox reaction of interest is driven by the bias in an exponential fashion, according to the Butler-Volmer relation. Moreover, owing to the minute levels of current encountered in amperometric, voltametric, and impedimetric in vivo analyte sensors, oftentimes in the picoampere range, high fidelity analog circuit design techniques is employed to quantize these current levels with femtoampere-level resolution. Thus, in one example, a precision bias circuit, known as a potentiostat, is implemented, along with a transimpedance amplifier offering high transconductance. In one example the combination of a precision bias circuit/potentiostat, along with a high transconductance transimpedance amplifier, collectively, is known as an amperometric (or potentiostatic) analog front end (AFE).

In one example, a high-resolution analog-to-digital converter is paired with this AFE system to quantize signals with sufficient resolution (e.g., 16 bits with high accuracy and low nonlinearity figures-of-merit). In one example, a poteniotstatic AFE comprising an ultra-high input impedance (Zin>100 GΩ) at the reference electrode input and an ultra-low input bias current (ibias)/input offset voltage (Voffset) (ibias<100 pA, Voffset≤10 mV) at the working electrode input can be used. The counter electrode terminal, in the case of a 3-terminal electrochemical sensing arrangement, in one example, provides sufficient compliance voltage to enable the potentiostat to source (or sink) a sufficient amount of current to sustain a redox reaction of interest at the working electrode. In one example, CMOS input instrumentation amplifiers, unity gain amplifiers, and differential amplifiers are used within the said circuits to achieve these performance requirements. A system that employs multiple potentiostats for the measurement of multiple analytes, can be configured to minimize of cross-talk between sensing channels (e.g., channel-to-channel isolation>75 dB). In one example, guard rings are also used in potentiostat designs to minimize noise that might result from high input impedance circuit couplings and thereby corrupt accurate low current measurements.

An exemplary dual potentiostat precision electrochemical analog front end commercially available from Analog Devices® is the Maxim Integrated® MAX30132. This integrated circuit is used in blood glucose meters and can be extended to the quantification of other analytes. The MAX30132 comprises guard rings and temperature compensation and is able to measure two analytes in parallel.

Guard rings are employed, in one example, to complement the design of a dual-channel AFE and to reduce capacitively-coupled noise that can perturb sensitive measurements of charge, e.g., when the charge magnitude is low. Moreover, noise can also be coupled between both channels, resulting in measurable signal cross-talk. Recalling from the canonical relation for charge stored in a capacitive system:

### Q=CV

The classic interpretation of this relation if voltage changes with time is often given by:

$$\frac{d Q ( t )}{dt} = i ( t ) = C \frac{dV ( t )}{dt}$$

The above relation may not account for time-dependent perturbations of the capacitive nature of the system. Thus, the product rule to form the following differential equation can be used:

$$\frac{dQ ( t )}{dt} = i ( t ) = \frac{dC ( t )}{dt} V ( t ) + C ( t ) \frac{dV ( t )}{dt}$$

Thus, a measured change in electrical charge, otherwise known as a current, is not only sensitive to a variation in electrical potential, but it is also affected due to change in the capacitive energy storage characteristics of the system (capacitive coupling). In one example, by driving a circumscribed conductor to the same potential as a signal trace on a PCB, leakage current is minimized, as the Ohmic drop between the conductors trends towards 0. For example, low leakage current can be achieved using a voltage buffer/follower that matches the guard voltage to the signal voltage, or in low-voltage differential sensing with an instrumentation amplifier, the common-mode voltage. The leakage from the guard ring to other circuit elements is typically of little concern as it is being sourced from a buffer which has a low output impedance.

In one example an additional sensing channel can be incorporated in an existing form-factor, e.g., a continuous glucose sensor (CGM) to enable the quantification of two metabolites, continuously and in real-time. Other form factors for the additional sensing channel can be used.

In one example, a suitable dual-channel amperometric analog front end for inclusion into a continuous analyte monitor wearable is used. Such a dual channel comprising a viable circuit topology meeting established performance requirements to achieve continuous amperometric quantification of two analytes is employed. In some examples, an amperometric analog front end (AFE)/bi-potentiostat amenable to power budget and size/footprint considerations is chosen. In other examples, an amperometric analog front end may feature an input for an external temperature sensor (e.g., thermistor or RTD) or may otherwise contain an internal bandgap-based temperature sensor. The reading from said temperature sensor may be used to compensate for increasing rates of enzyme turnover with temperature, in accordance with the Arrhenius relation.

In another example, alone or in combination with the above exemplary analog front end (AFE), a printed circuit board (PCB) design and/or printed circuit board assembly (PCBA) supporting the dual-channel amperometric AFE and the dual coaxial wire sensing construct is provided. In one example, the PCBA is populated with the necessary components for a functional transmitter with dual-channel amperometric sensing capability. PCB design can incorporate an effective guard ring topology enabling low-noise amperometric measurements and can feature a common reference/counter electrode configuration for both sensing channels.

In one example, alone or in combination with the PCB, PCBA, AFE, the dual-wire coaxial sensor construct is adapted to (e.g., embedded into) an existing CGM form-factor or another form factor. In another example, the selection of materials for the form-factor can be used to mitigate ingress of water, dust, and/or light. The said materials can also be selected to present low outgassing or vapor/water uptake figures of merit. The said materials can also be selected to possess high linear, sheet, or volume resistivity figures of merit (ohm-cm) to eliminate leakage currents or signal cross-talk between channels and electrodes.

In another example, alone or in combination with the above AFE, the printed circuit board (PCB) design and printed circuit board assembly (PCBA) supporting the dual-channel amperometric AFE and the dual coaxial wire sensing construct is coupled with firmware supporting dual-channel amperometric measurements at configurable data storage intervals. In one example, existing firmware of a continuous analyte monitor is modified to enable power-efficient control of the amperometric AFE and data transmission, for example, over a Bluetooth Low Energy connection. In another example, the firmware modification of existing firmware to enable extended data storage in an embedded flash memory IC and transmission of readings to a real-time display is provided.

Thus, in one example, a power-efficient firmware enabling dual-channel amperometric measurements at pre-defined data storage intervals, with amperometric measurements (both channels) recordable in an embedded device memory for at least the full intended operational lifetime (15 days) of the transmitter is provided.

In one example, the dual-channel amperometric sensing coupled to the transmitter PCBA paired with a dual coaxial wire sensor configuration in vitro, provides data capable of being logged in embedded device memory and transmitted to a paired real-time display for at least 7 days, 8 days, 9 days, 10 days, 11 days, 12 days, 13 days, 14 days, 15 days, 21 days, or over 21 days.

In one example, the dual analyte transmitter, paired with a dual coaxial sensor configuration measuring two channels of glucose information, in an animal model, shows 15.5-day quantitative tracking of interstitial glucose levels, as assessed by a comparator measure in arterialized blood samples.

In one example, the amperometric analog front end supporting the multi-analyte sensing platform supports one or more potentiostats, temperature correction, configurable bias conditions, and advanced electrochemistry for multi-analyte sensing. In one example, guard ring topology enabling low-noise amperometric measurements is employed.

By way of example, the multi-analyte transmitter, paired with a dual coaxial sensor contingent measuring two channels of glucose information, in an animal model, is likely to show 15.5-day quantitative tracking of interstitial glucose levels, as assessed by a comparator measure in arterialized blood samples. In one example, the dual coaxial sensor configuration can be tested using glucose with another analyte, e.g., ketone.

In some examples, the potentiostat includes a resistor that translates the current into voltage. In some alternate examples, a current to frequency converter is provided that is configured to continuously integrate the measured current, for example, using a charge counting device. An A/D converter digitizes the analog signal into a digital signal, also referred to as “counts” for processing. Accordingly, the resulting raw data stream in counts, also referred to as raw sensor data, is directly related to the current measured by the potentiostat.

A processor module includes the central control unit that controls the processing of the sensor electronics. In some examples, the processor module includes a microprocessor, however a computer system other than a microprocessor can be used to process data as described herein, for example an ASIC can be used for some or all of the sensor's central processing. The processor typically provides semi-permanent storage of data, for example, storing data such as sensor identifier (ID) and programming to process data streams (for example, programming for data smoothing and/or replacement of signal artifacts such as is described in U.S. Pat. No. 8,010,174 to Goode et al. The processor additionally can be used for the system's cache memory, for example for temporarily storing recent sensor data. In some examples, the processor module comprises memory storage components such as ROM, RAM, dynamic-RAM, static-RAM, non-static RAM, EEPROM, rewritable ROMs, flash memory, or the like.

In some examples, the processor module comprises a digital filter, for example, an IIR or FIR filter, configured to smooth the raw data stream from the A/D converter. Generally, digital filters are programmed to filter data sampled at a predetermined time interval (also referred to as a sample rate). In some examples, wherein the potentiostat is configured to measure the analyte at discrete time intervals, these time intervals determine the sample rate of the digital filter. In some alternate examples, wherein the potentiostat is configured to continuously measure the analyte, for example, using a current-to-frequency converter as described above, the processor module can be programmed to request a digital value from the A/D converter at a predetermined time interval, also referred to as the acquisition time. In these alternate examples, the values obtained by the processor are advantageously averaged over the acquisition time due the continuity of the current measurement. Accordingly, the acquisition time determines the sample rate of the digital filter. In one example, the processor module is configured with a programmable acquisition time, namely, the predetermined time interval for requesting the digital value from the A/D converter is programmable by a user within the digital circuitry of the processor module. In one example, an acquisition time of from about 2 seconds to about 512 seconds is used; however, any acquisition time can be programmed into the processor module. A programmable acquisition time is advantageous in maximizing the usefulness of, for example, noise filtration, time lag, and/or processing/battery power.

In some examples, the processor module is configured to build the data packet for transmission to an outside source, for example, an RF transmission to a receiver as described in more detail below. Generally, the data packet comprises a plurality of bits that can include a sensor ID code, raw data, filtered data, and/or error detection or correction. The processor module can be configured to transmit any combination of raw and/or filtered data.

In some examples, the processor module further comprises a transmitter portion that is programmable and programmed for a transmission interval of the sensor data to a receiver, or the like. In some examples, the transmitter portion, which is programmable for the interval of transmission. In one such example, a coefficient can be chosen (e.g., a number of from about 1 to about 100, or more), wherein the coefficient is multiplied by the acquisition time (or sampling rate), such as described above, to define the transmission interval of the data packet. Thus, in some examples, the transmission interval is programmable between about 2 seconds and about 850 minutes. In further examples, the transmission interval is programmable between about 30 second and 5 minutes. Nevertheless, any transmission interval can be programmable or programmed into the processor module. A variety of alternate systems and methods for providing a programmable transmission interval can also be employed. By providing a programmable transmission interval, data transmission can be customized to meet a variety of design criteria (e.g., reduced battery consumption, timeliness of reporting sensor values, etc.). In some examples, a transceiver module can also be included in the sensor electronics. In one example, the transceiver module may be configured to transmit and/or receive sensor data.

The various memories and/or memory of the processor unit(s) and/or storage device may store one or more sets of instructions and data structures (e.g., instructions) embodying or used by any one or more of the methodologies or functions described herein. These instructions, when executed by processor unit(s) cause various operations to implement the disclosed examples. The instructions can further be transmitted or received over a communications network using a transmission medium via the network interface device using any one of several well-known transfer protocols (e.g., HTTP). Examples of communication networks include a LAN, a WAN, the Internet, mobile telephone networks, plain old telephone service (POTS) networks, and wireless data networks (e.g., Wi-Fi, 3G, 4G LTE/LTE-A, 5G, or WiMAX networks). The term “transmission medium” shall be taken to include any intangible medium that is capable of storing, encoding, or carrying instructions for execution by the machine, and includes digital or analog communications signals or other intangible media to facilitate communication of such software.

Conventional glucose sensors measure current in the nanoAmp range. In contrast to conventional glucose sensors, the presently disclosed multi-analyte sensors are configured to measure the current flow in the picoAmp range, and in some examples, femtoAmps, if required. Namely, for every unit (mg/dL) of glucose measured, at least one picoAmp of current is measured. In some examples, the analog portion of the A/D converter is configured to continuously measure the current flowing at the working electrode and to convert the current measurement to digital values representative of the current. In one example, the current flow is measured by a charge counting device (e.g., a capacitor). Thus, a signal is provided, whereby a high sensitivity maximizes the signal received by a minimal amount of measured hydrogen peroxide (e.g., minimal glucose requirements without sacrificing accuracy even in low glucose ranges), reducing the sensitivity to oxygen limitations in vivo (e.g., in oxygen-dependent glucose sensors).

A battery is operably connected to the sensor electronics and provides the power for the sensor. In one example, the battery is a lithium manganese dioxide battery; however, any appropriately sized and powered battery can be used (for example, AAA, nickel-cadmium, zinc-carbon, alkaline, lithium, nickel-metal hydride, lithium-ion, zinc-air, zinc-mercury oxide, silver-zinc, and/or hermetically-sealed). In some examples, the battery is rechargeable, and/or a plurality of batteries can be used to power the system. The sensor can be transcutaneously powered via an inductive coupling, for example. In some examples, a quartz crystal is operably connected to the processor and maintains system time for the computer system as a whole, for example, for the programmable acquisition time within the processor module.

An optional temperature probe can be provided, wherein the temperature probe is located on the electronics assembly or the glucose sensor itself. The temperature probe can be used to measure ambient temperature in the vicinity of the glucose sensor. This temperature measurement can be used to add temperature compensation to the calculated glucose value.

An RF module is operably connected to the processor and transmits the sensor data from the sensor to a receiver within a wireless transmission via antenna or other wireless communication methods. In some examples, a second quartz crystal provides the time base for the RF carrier frequency used for data transmissions from the RF transceiver. In some alternate examples, however, other mechanisms, such as optical, infrared radiation (IR), ultrasonic, or the like, can be used to transmit and/or receive data.

In the RF telemetry module of the present disclosure, the hardware and software are designed for low power requirements to increase the longevity of the device (for example, to enable a life of from about 3 to about 24 months, or more) with maximum RF transmittance from the in vivo environment to the ex vivo environment for some implantable sensors (for example, a distance of from about one to ten meters or more). In some examples, a high frequency carrier signal of from about 402 MHz to about 433 MHz is employed in order to maintain lower power requirements. Additionally, in some implantable devices, the carrier frequency is adapted for physiological attenuation levels, which is accomplished by tuning the RF module in a simulated in vivo environment to ensure RF functionality after implantation; accordingly, the glucose sensor can sustain sensor function for 3 months, 6 months, 12 months, or 24 months or more.

In some examples, output signal (from the sensor electronics) is sent to a receiver (e.g., a computer or other communication station). The output signal is typically a raw data stream that is used to provide a useful value of the measured analyte concentration to a patient or a doctor, for example. In some examples, the raw data stream can be continuously or periodically algorithmically smoothed or otherwise modified to diminish outlying points that do not accurately represent the analyte concentration, for example due to signal noise or other signal artifacts, such as described in U.S. Pat. No. 6,931,327 to Goode et al., which is incorporated herein by reference in its entirety.

When a sensor is first implanted into host tissue, the sensor and receiver are initialized. This can be referred to as start-up mode and involves optionally resetting the sensor data and calibrating the sensor. In selected examples, mating the electronics unit to the mounting unit triggers a start-up mode. In other examples, the start-up mode is triggered by the receiver.

### Receiver

In some examples, the sensor electronics are wirelessly connected to a receiver via one- or two-way RF transmissions or the like. However, a wired connection is also contemplated. The receiver provides much of the processing and display of the sensor data and can be selectively worn and/or removed at the host's convenience. Thus, the sensor system can be discreetly worn, and the receiver, which provides much of the processing and display of the sensor data, can be selectively worn and/or removed at the host's convenience. Particularly, the receiver includes programming for retrospectively and/or prospectively initiating a calibration, converting sensor data, updating the calibration, evaluating received reference and sensor data, and evaluating the calibration for the analyte sensor, such as described in more detail with reference to U.S. Pat. No. 7,778,680 to Goode et al.

U.S. Pat. No. 7,134,999 to Brauker et al. describes systems and methods suitable for the sensor body and is incorporated herein by reference in its entirety. In one example, a biointerface membrane is formed onto the sensing mechanism as described in more detail elsewhere herein. The sensor body includes sensor electronics and communicates with a receiver as described in more detail, above. A drug releasing membrane can be disposed on at least a portion of biointerface membrane and/or sensing mechanism.

In certain examples, the sensing device, which is implantable into the host, such as in the soft tissue beneath the skin, is implanted subcutaneously, such as in the abdomen of the host, for example. A person of ordinary skill in the art appreciates a variety of suitable implantation sites available due to the sensor's small size. In some examples, the sensor architecture is less than about 0.5 mm in at least one dimension, for example a wire-based sensor with a diameter of less than about 0.5 mm. In another exemplary example, for example, the sensor may be 0.5 mm thick, 3 mm in length and 2 cm in width, such as possibly a narrow substrate, needle, wire, rod, sheet, or pocket. In another exemplary example, a plurality of about 1 mm wide wires about 5 mm in length could be connected at their first ends, producing a forked sensor structure. In still another example, a 1 mm wide sensor could be coiled, to produce a substantially planar, spiraled sensor structure. Although a few examples are cited above, numerous other useful examples are contemplated by the present disclosure, as is appreciated by a person of ordinary skill in the art.

Post implantation, a period of time is allowed for tissue ingrowth within the biointerface membrane. The length of time required for tissue ingrowth varies from host to host, such as about a week to about 3 weeks, although other time periods are also possible. Once a mature bed of vascularized tissue has grown into the biointerface membrane, a signal can be detected from the sensor, as described elsewhere herein and in U.S. Pat. Appl. Pub. No. 2005/0245799 to Brauker et al., which is incorporated herein in its entirety. Long term sensors can remain implanted and produce glucose signal information from months to years, as described in the above-cited patent application.

In certain examples, the device is configured such that the sensing unit is separated from the electronics unit by a tether or cable, or a similar structure. A person of ordinary skill in the art will recognize that a variety of known and useful means may be used to tether the sensor to the electronics. While not wishing to be bound by theory, it is believed that the FBR to the electronics unit alone may be greater than the FBR to the sensing unit alone, due to the electronics unit's greater mass, for example. Accordingly, separation of the sensing and electronics units effectively reduces the FBR to the sensing unit and results in improved device function. As described elsewhere herein, the architecture and/or composition of the sensing unit (e.g., inclusion of a drug releasing membrane with certain bioactive agents) can be implemented to further reduce the foreign body response to the tethered sensing unit.

In another example, an analyte sensor is designed with separate electronics and sensing units, wherein the sensing unit is inductively coupled to the electronics unit. In this example, the electronics unit provides power to the sensing unit and/or enables communication of data therebetween.

In yet another example, the implanted sensor additionally includes a capacitor to provide necessary power for device function. A portable scanner (e.g., wand-like device) is used to collect data stored on the circuit and/or to recharge the device.

In general, inductive coupling, as described herein, enables power to be transmitted to the sensor for continuous power, recharging, and the like. Additionally, inductive coupling utilizes appropriately spaced and oriented antennas (e.g., coils) on the sensing unit and the electronics unit so as to efficiently transmit/receive power (e.g., current) and/or data communication therebetween. One or more coils in each of the sensing and electronics unit can provide the necessary power induction and/or data transmission.

In this example, the sensing mechanism can be, for example, a wire-based sensor as described in more detail as described in U.S. Pat. No. 7,497,827 to Brister et al., or a planar or substantially planar substrate-based sensor such as described in U.S. Pat. No. 6,175,752 to Say et al. and U.S. Pat. No. 5,779,665 to Mastrototaro et al., all of which are incorporated herein by reference in their entirety.

### Sterilization

In one example, the devices disclosed herein are sterilized. In one example, the devices disclosed herein are sterilized using high energy radiation, for example, e-beam, x-ray, or ultraviolet light. In another example, the devices disclosed herein are sterilized using ethylene oxide. In some pyridine polymers, ethylene oxide sterilization can cause swelling and discoloration. Thus, in one example, one or more of the membranes of the sensor system comprise PVPy and/or copolymers thereof and are modified prior to exposure to ethylene oxide. For example, one or more of the pyridine functionalities of the PVPy polymer and/or copolymers thereof are quarternized as shown in Scheme 2.

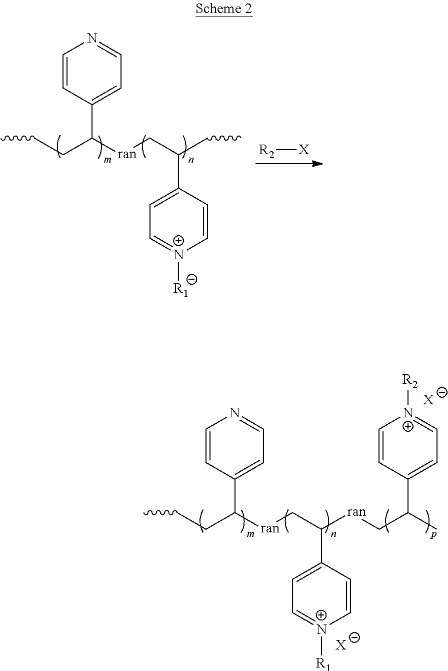

where m and n>0; R1 is a crosslinking moiety; “ran” is a carbon-carbon bond or copolymer unit; p≤m; R2 is alkyl, benzyl, aryl, halide-end group polymers selected from polysiloxanes, polyethers, polyethylene ethers, polyethylene-polypropylene ethers, polycarbonates or poly zwitterionic polymers; and X is any leaving group suitable for alkylation, e.g., bromide, chloride, sulfonate, weak bases.

In one example, essentially all of the pyridine functionalities of the PVPy polymer and copolymers thereof are quarternized, e.g., m=p in Scheme 2. In one example, pyridine functionalities of the PVPy polymer and/or copolymers thereof are quarternized prior to being disposed on the sensor substrate. In another example, pyridine functionalities of the PVPy polymer and/or copolymers thereof are quarternized after being disposed on the sensor substrate. In one example, the pyridine functionalities of the PVPy polymer and/or copolymers thereof are quarternized using an alklyating agent R2. In one example, the pyridine functionalities of the PVPy polymer and/or copolymers thereof are quarternized, for example, using the Menshutkin reaction with an alkylating agent R2. In one example, the pyridine functionalities of the PVPy polymer and/or copolymers thereof are quarternized, for example, using the Menshutkin reaction with an alkylating agent selected from alkyl halides (“halide” includes iodide, bromide, or chloride), alkyl halides with an ionic moiety, benzyl halides, benzyl halides with an ionic moiety, or halide-end group polymers selected from polysiloxanes, polyethers, polyethylene ethers, polyethylene-polypropylene ethers, polycarbonates or poly zwitterionic polymers. In one example, the halide end group polymer has a low molecular weight, for example, 100-1000 Daltons. In one example, the partially alkylated PVPy and/or copolymers can be subjected to ion exchange to replace the halide with another anion.

Sterilization using ethylene oxide of the devices disclosed herein comprising PVPy polymers or copolymers having alkylated or alkylpolyol/quarternized pyridine functionalities provide reduction or elimination of swelling and/or discoloration, improvement in sensor break in, sensor sensitivity, and sensor lifetime, among other improvements, compared to ethylene oxide sterilized PVPy polymers or copolymers having pyridine functionalities that are not quarternized or only partially quarternized. In one example, the degree of quaternization is less than 50, 40, 30, or less than 25 mole % of the moles of pyridine present in the polymer. In one example, the degree of quaternization is greater than 50, 60, 70, 80, or greater than 90 mole % of the moles of pyridine present in the polymer.

In one example, one or more of the membranes of the sensor system comprise PVPy and/or copolymers thereof and are partially cross-linked with crosslinking moiety R1 and partially alkylated to provide partially quarternized pyridine groups. In one example, R1 is a polycarbodiimide crosslinker. In one example, one or more of the membranes of the sensor system comprise PVPy and/or copolymers thereof and are partially cross-linked and partially alkylated using a molar ratio of alkylating agent that is less than the total amount of moles of pyridine present in the polymer. In one example, one or more of the membranes of the sensor system comprise PVPy and/or copolymers thereof and are partially alkylated using a molar ratio of alkylating agent that is less than the total amount of moles of pyridine present in the polymer prior to deposition on a sensor substrate and are subsequently cross-linked as a membrane (e.g., resistance membrane). In another example, one or more of the membranes of the sensor system comprise PVPy and/or copolymers thereof and are partially alkylated using a molar ratio of alkylating agent that is less than the total amount of moles of pyridine present in the polymer subsequently cross-linked prior to deposition on a sensor substrate as a membrane (e.g., resistance membrane). The above partially cross-linked and partially alkylated PVPy and/or copolymers can be used as membranes in devices disclosed herein that are sterilized using ethylene oxide.

In one example, the above partially alkylated PVPy and/or copolymers can be used to retain or inhibit migration of cofactor, for example, NAD(H), or enzyme, within one or more membranes or layers. In one example, the above partially alkylpolyol-PVPy and/or copolymers can be used to retain or inhibit migration of cofactor, for example, NAD(H), or enzyme, within one or more membranes or layers.

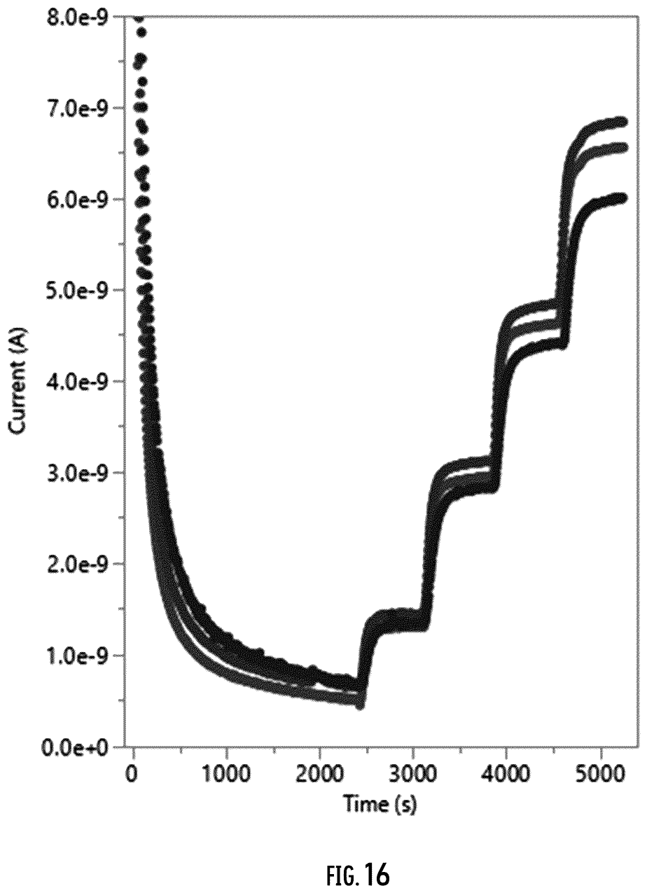

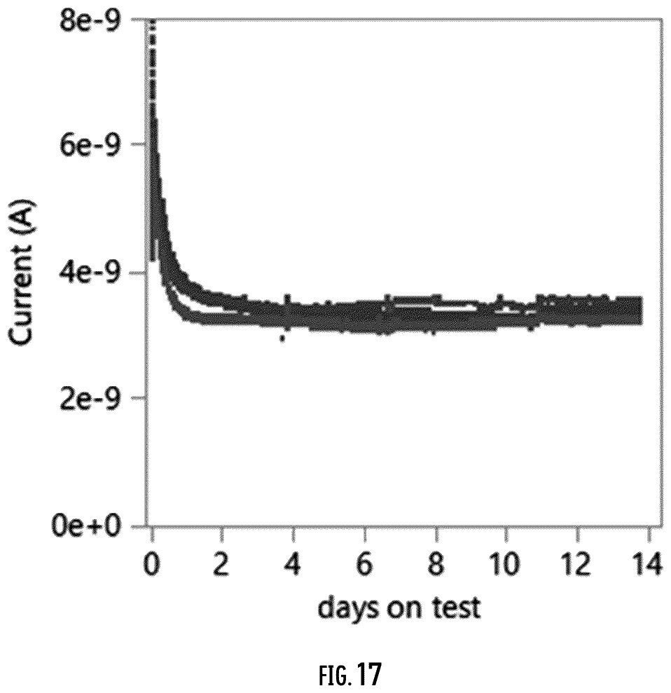

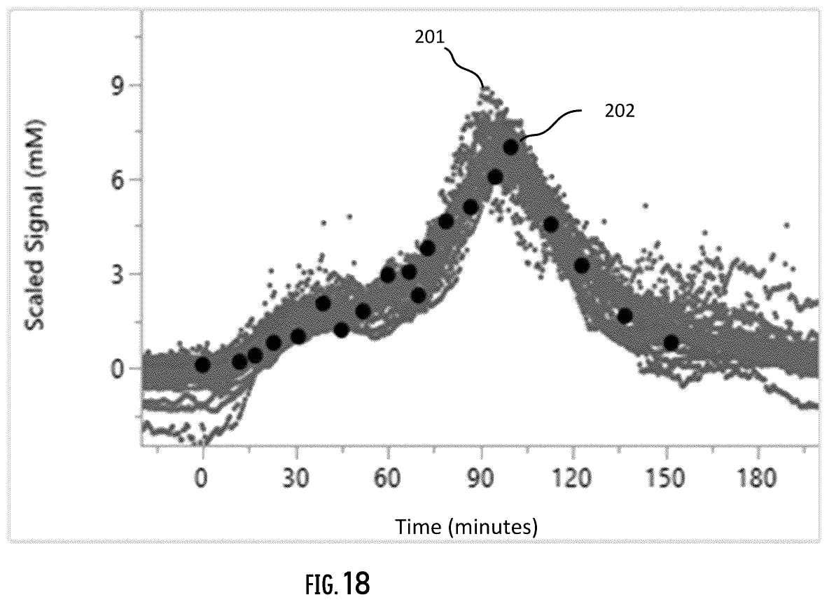

With reference to FIG. 16, exemplary continuous ketone sensor data is shown comprising a gold wire WE biased @0.2V vs Ag/AgCl, without an interference layer, and an enzyme domain of PVI-Os(bpy)2Cl mediator, NAD+, HBDH, and diaphorase in a crosslinked PEG-DGE matrix. An amphiphilic polyurethane resistance domain was deposited over the enzyme domain. The data of FIG. 16 shows calibration data from titrations of ketone (0-7 mmol) that demonstrate good sensitivity. The data of FIG. 17 shows drift data of the sensor for 14 days that demonstrates good stability. The data of FIG. 18 shows continuous in vivo (pig) excursion model data 201 compared with externally obtained reference data 202, demonstrating good in vivo correlation.

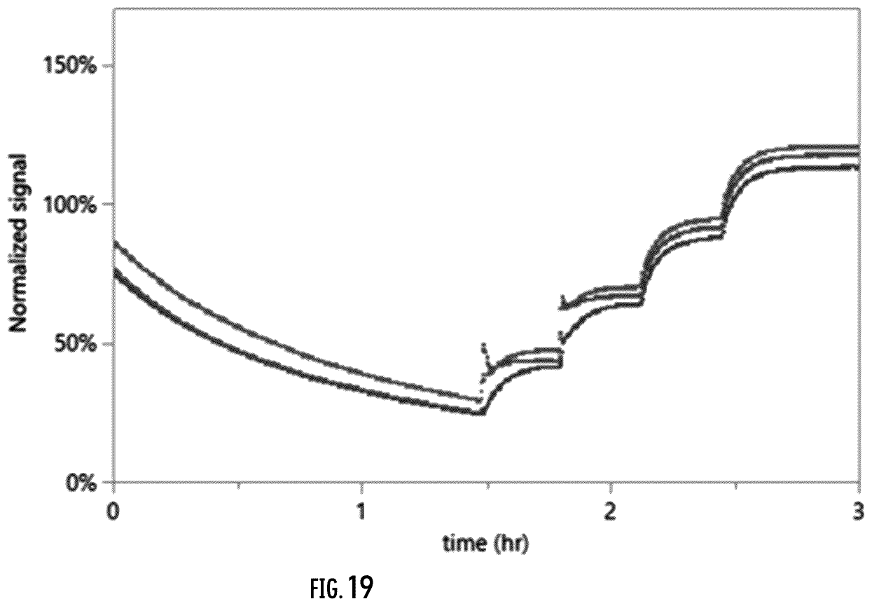

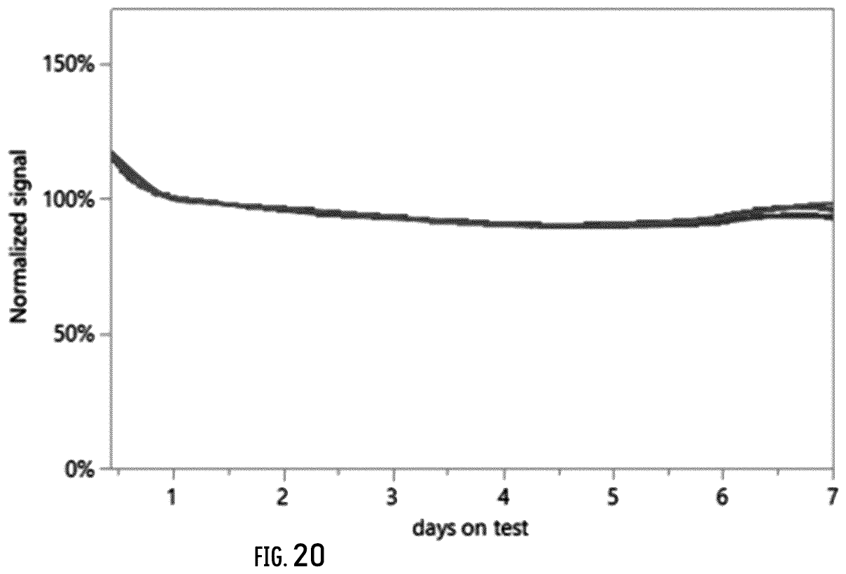

With reference to FIG. 19, data from identically prepared exemplary non-mediated continuous ketone sensor comprising a platinum wire WE biased @0.6V vs Ag/AgCl, an interference domain, and an enzyme domain of NAD, HBDH, and NADH oxidase in a water dispersible polyurethane-zwitterion polymer crosslinked with polycarbodiimide is shown. A resistance domain of poly vinylpyridine with PEG-DGE was deposited over the enzyme domain and crosslinked. The data of FIG. 19 shows calibration data of the aforementioned sensor from titrations of ketone that demonstrates good sensitivity. The data of FIG. 20 shows drift data of the aforementioned sensor for 7 days that demonstrates good stability.

The above description discloses several methods and materials of the present disclosure. This disclosure is susceptible to modifications in the methods and materials, as well as alterations in the fabrication methods and equipment. Such modifications will become apparent to a person of ordinary skill in the art from a consideration of this disclosure or practice of the disclosure disclosed herein. Consequently, it is not intended that this disclosure be limited to the specific examples disclosed herein, but that it cover all modifications and alternatives coming within the true scope and spirit of the disclosure.

While certain examples of the present disclosure have been illustrated with reference to specific combinations of elements, various other combinations may also be provided without departing from the teachings of the present disclosure. Thus, the present disclosure should not be construed as being limited to the particular exemplary examples described herein and illustrated in the Figures, but may also encompass combinations of elements of the various illustrated examples and aspects thereof.

1-78. (canceled)

79. A continuous analyte sensor device comprising:
an analyte sensor operably coupled to a signal transducer, the analyte sensor comprising at least one membrane system adjacent the signal transducer, the least one membrane system comprising, independently:
a first domain comprising at least one first transducing element;
a second domain adjacent the first domain, the second domain being the same or different as the first domain; and
at least one regenerative cofactor.

80. The continuous analyte sensor device of claim 79, wherein the analyte sensor is a multi-analyte sensor.

81. The continuous analyte sensor device of claim 80, wherein the multi-analyte analyte sensor is a glucose and ketone analyte sensor, or a glucose and creatinine analyte sensor, or a ketone and potassium ion analyte sensor.

82. The continuous analyte sensor device of claim 79, wherein the signal transducer comprises at least one electrode.

83. The continuous analyte sensor device of claim 79, wherein the first domain comprises at least one first transducing element and the second domain comprises at least one second transducing element, the second transducing element being different from the first transducing element.

84. The continuous analyte sensor device of claim 79, wherein the at least one regenerative cofactor is one or more of NAD, NADH, NAD(P)H, NAD(P)+, ATP, flavin adenine dinucleotide (FAD), magnesium (Mg++), pyrroloquinoline quinone (PQQ), pyrroloquinoline quinone (PQQ), and functionalized derivatives thereof.

85. The continuous analyte sensor device of claim 79, further comprising at least one mediator present in the first domain, the second domain, or in both the first and second domains.

86. The continuous analyte sensor device of claim 85, wherein the mediator is one or more of 2,2′-bipryidine, poly-1,10-phenanthroline-5,6-dione, polyvinylferrocene, hexacyanoferrate, phthalocyanine or organometallic compounds thereof, organometallic compounds of osmium or ruthenium, and functionalized derivatives thereof, and complexes of one or more transition metals with one or more polymers or ligands and salts thereof.

87. The continuous analyte sensor device of claim 85, wherein the first domain comprises the at least one mediator and the at least one first transducing element, and the second domain comprises the at least one regenerative cofactor, or wherein the first domain comprises the at least one regenerative cofactor and the at least one first transducing element and the second domain comprises the at least one mediator.

88. The continuous analyte sensor device of claim 83, wherein the at least one first transducing element and the at least one second transducing element are independently a dehydrogenase enzyme, a reductase enzyme, a kinase enzyme, a peroxidase enzyme, an esterase enzyme, an (amido)hydrolase enzyme, an oxidase enzyme, or combinations thereof.

89. The continuous analyte sensor device of claim 83, wherein the at least one first transducing element and the at least one second transducing element are independently beta-hydroxybutyrate dehydrogenase, alcohol dehydrogenase, lipase, amidohydrolase, glycerol kinase, creatinine kinase, creatine amidohydrolase, alcohol oxidase, cholesterol oxidase, galactose oxidase, choline oxidase, glutamate oxidase, glycerol-3-phosphate oxidase, bilirubin oxidase, urease, pyruvate oxidase, xanthine oxidase, glucose oxidase, lactate oxidase, sarcosine oxidase, malate dehydrogenase, formaldehyde dehydrogenase, glutathione reductase, glutathione peroxidase, 3-hydroxy steroid dehydrogenases, NADH oxidase, or combinations thereof.

90. The continuous analyte sensor device of claim 83, wherein the at least one first transducing element is beta-hydroxybutyrate dehydrogenase, and the at least one second transducing element is NADH oxidase.

91. The continuous analyte sensor device of claim 82, wherein:
the at least one electrode comprises platinum or palladium;
an interference domain deposited on the at least one electrode;
the first domain is adjacent the interference domain, the first domain comprising beta-hydroxybutyrate dehydrogenase, NADH oxidase, and the regenerative cofactor; and
the second domain adjacent the first domain, the second domain comprising poly vinylpyridine polymer or copolymer;
wherein the continuous analyte sensor device is configured to provide a continuous analyte signal without a transition metal-containing mediator.

92. The continuous analyte sensor device of claim 91, wherein the at least one regenerative cofactor is NAD.

93. The continuous analyte sensor device of claim 91, wherein the interference domain is configured to block diffusion of at least one of acetaminophen, ascorbic acid, bilirubin, cholesterol, creatinine, dopamine, ephedrine, ibuprofen, L-dopa, methyldopa, salicylate, tetracycline, tolazamide, tolbutamide, triglycerides, and uric acid from the electrode surface.

94. The continuous analyte sensor device of claim 91, wherein the interference domain comprises polyurethanes, polyurethane-zwitterion polymers, polymers having pendant ionic groups, NAFION™, chitosan, cellulose, alternating layers of polyallylamine and polyacrylate acid or combinations or blends thereof.

95. The continuous analyte sensor device of claim 82, wherein the first domain or the second domain comprises an amphiphilic polymer or copolymer.

96. The continuous analyte sensor device of claim 82, wherein the first domain or the second domain comprises a heterocyclic polymer or copolymer, or an at least partially quarternized heterocyclic polymer or copolymer.

97. The continuous analyte sensor device of claim 79, further comprising a transmitter configured to wireless transmit data to a paired display device or therapeutic delivery device.

98. The continuous analyte sensor device of claim 79, wherein the continuous analyte sensor device is sterilized.
